(19) United States
(12) Patent Application Publication
(10) Pub. No.: US 2020/0060262 A1
(43) Pub. Date: Feb. 27, 2020

(54) METHODS TO REDUCE CLOT FORMATION IN COLD-STORED PLATELET PRODUCTS

(71) Applicant: BLOODWORKS, Seattle, WA (US)

(72) Inventor: Moritz Stolla, Seattle, WA (US)

(73) Assignee: BLOODWORKS, Seattle, WA (US)

(21) Appl. No.: 16/549,799

(22) Filed: Aug. 23, 2019

Related U.S. Application Data
(60) Provisional application No. 62/722,600, filed on Aug. 24, 2018.

(51) Int. Cl. A01N 1/02; C12N 5/078

(52) U.S. Cl. CPC A01N 1/0284; C12N 5/0644

(57) ABSTRACT

Methods to significantly reduce clot formation in cold-stored platelet samples are described. The methods include collecting platelet samples at defined yields and/or concentrations and allowing collected platelet samples to rest at room temperature without agitation for a period of time before being moved into cold storage.

### CROSS-REFERENCE TO RELATED APPLICATION

This application claims the benefit of priority to U.S. Provisional Patent Application No. 62/722,600, filed on Aug. 24, 2018, which is incorporated by reference in its entirety as if fully set forth herein.

### STATEMENT REGARDING FEDERALLY SPONSORED RESEARCH OR DEVELOPMENT

This invention was made with government support under grant W81XWH-12-0281 awarded by the Department of Defense. The government has certain rights in the invention.

### FIELD OF THE DISCLOSURE

The current disclosure provides methods to significantly reduce clot formation in cold-stored platelet samples. The methods include collecting platelet samples at defined yields and/or concentrations and allowing collected platelet samples to rest at room temperature without agitation for a period of time before being into cold storage.

### BACKGROUND OF THE DISCLOSURE

Whole blood contains various cellular components, such as red blood cells (RBC), platelets and white blood cells, suspended in a liquid plasma component. Donations of whole blood often are separated into the individual, clinically therapeutic components for individual storage and use in treating medical conditions that require administration of one or more particular blood components to a patient.

Platelets are non-nucleated bone marrow-derived blood cells that protect injured mammals from blood loss by adhering to sites of vascular injury and by promoting the formation of plasma fibrin clots. Humans depleted of circulating platelets by bone marrow failure suffer from life threatening spontaneous bleeding, and less severe deficiencies of platelets contribute to bleeding complications following trauma or surgery.

As the count of circulating platelets falls, patients become increasingly susceptible to cutaneous bleeding. Patients with platelet counts of less than 10,000 per μL are highly susceptible to spontaneous hemorrhage, especially when the low platelet count is caused by a bone marrow disorder or failure (e.g., aplastic anemia, acute and chronic leukemia, metastatic cancer, and deficiencies resulting from cancer treatment such as ionizing radiation or chemotherapy).

A major advance in medical care half a century ago was the development of platelet transfusions to correct platelet deficiencies. Currently, there are an estimated 2.6 million platelet transfusions in the United States per year.

Platelets for clinical use are currently stored at room temperature. Room temperature storage is based on discoveries made during the 1960s that cold-storage (e.g., at refrigerated temperatures) leads to a significant reduction in platelet survival in recipients.

The need to keep platelets at room temperature prior to transfusion has imposed a unique set of costly and complex logistical requirements on platelet storage. Because platelets are metabolically active at room temperature, they require constant agitation in gas permeable containers to allow for the exchange of gases to prevent the toxic consequences of metabolic acidosis. Room temperature storage conditions result in macromolecular degradation and reduced hemostatic activity. Hemostatic activity broadly refers to the ability of a population of platelets to mediate bleeding cessation. The observed macromolecular degradation and reduced hemostatic activity of stored platelets are collectively referred to as the “platelet storage lesion” (PSL). In addition, storage at room temperature encourages the growth of bacteria. In this regard, bacterial contamination of platelets is by far the most frequent infectious complication of blood component use. At current rates, from one in 1,000 to one in 2,000 units of platelets are contaminated with bacteria at a level sufficient to pose a significant risk to the recipient.

Platelet storage at refrigerated temperatures has potential benefits, including better hemostatic function and a lowered risk of infectious diseases. Based on this, FDA-approval for the cold-storage of platelets has been sought. Stubbs et al., Transfusion, 57, 2836-2844 (December 2017). However, in this study, while various benefits related to cold-storage were achieved, major challenges were noted including a high degree of product wastage. One reason for the high wastage included macro-aggregate clot formation within the stored samples. More particularly, macro-aggregate clot formation was observed in 18.2% of samples. Stubbs et al. concluded, “[i]f these limitations could be addressed, [cold-stored platelets] would become a much more practical option for bleeding patients.” Page 2842, 2^{nd} column. Stubbs et al., also stated that cold-stored platelets “with a shelf-life of 10 or more days would allow an expanded inventory of the blood product, and it could very well become the component of choice for all actively bleeding patients.” Page 2843, 1^{st} column.

### SUMMARY OF THE DISCLOSURE

The current disclosure provides methods for cold-storage of platelet samples with significantly reduced clot formation. In fact, the methods disclosed herein reduced macro-aggregate clot formation from the 18.2% observed in Stubbs et al., Transfusion, 57, 2836-2844 (December 2017) down to less than 3%. This significant decrease allows for better storage of platelet samples, allowing a much-needed expansion of the inventory of this important blood product.

In particular embodiments, the methods include one or more of the following characteristics: (i) collecting the platelets within plasma; (ii) collecting the platelets within a defined yield/bag; (iii) calibrating the concentration of platelets within plasma; (iv) allowing the collected sample to rest at room temperature before cooling; and/or (v) allowing the collected sample to rest at room temperature without agitation before cooling.

In particular embodiments, platelets are collected by apheresis.

In particular embodiments, the defined yield/bag is 2.0×10¹¹-4.5×10¹¹ platelets/bag. In particular embodiments, the defined yield/bag is 3.0×10¹¹-4.0×10¹¹ platelets/bag.

In particular embodiments, the concentration of platelets within plasma is calibrated to 0.5×10⁶-2.3×10⁶ platelets/μL plasma. In particular embodiments, the concentration of platelets within plasma is calibrated to 0.7×10⁶-2.1×10⁶ platelets/μL plasma. In particular embodiments, the concentration of platelets within plasma is calibrated to 1.5×10⁶ platelets/μL plasma.

In particular embodiments, platelet samples remain at room temperature for at least 20 minutes before cooling to 4±2° C. In particular embodiments, platelet samples remain at room temperature for 20 minutes to 6 hours before cooling to 4±2° C. In particular embodiments, platelet samples remain at room temperature for 20 minutes to 4 hours before cooling to 4±2° C. In particular embodiments, platelet samples remain at room temperature for 20 minutes to 2 hours before cooling to 4±2° C. In particular embodiments, platelet samples remain at room temperature for 1 hour before cooling to 4±2° C.

In particular embodiments, platelet samples remain at room temperature for at least 20 minutes without agitation, for 20 minutes to 6 hours without agitation, for 20 minutes to 4 hours without agitation, for 20 minutes to 2 hours without agitation, or for 1 hour without agitation before cooling to 4±2° C.

In particular embodiments, platelets are (i) collected into plasma utilizing apheresis; (ii) collected into a bag with a yield of 3.0×10¹¹-4.0×10¹¹ platelets/bag; (iii) calibrated to a concentration of 0.7×10⁶-2.1×10⁶ platelets/μL plasma; and (iv) allowed to rest at room temperature for 1 hour without agitation before being transferred to cold storage at 4±2° C. without agitation.

### BRIEF DESCRIPTION OF THE SEVERAL VIEWS OF THE DRAWINGS

FIGS. 1A, 1B: Platelet Yields of Apheresis Units: Platelets were stored in either plasma alone (Plasma, black circles data were 3 day control units collected during 10 day plasma stored studies and downward triangles data were from 3 day control units collected during 15 day plasma stored studies), or in a 65% platelet additive solution (PAS), 35% plasma mixture (Intersol, black squares, or Isoplate, black, upward triangles). [Total platelet yield in the component, calculated by multiplying the platelet count of the sample times the volume of the component (platelet count×component volume=actual platelet yield)]: (FIG. 1A) Platelets were stored for 3 days at 4° C. Results are shown as percentage of day 1 platelet counts. No significant differences were seen between the groups. (FIG. 1B) 10 day and 15 day cold-stored platelets (CSP; as indicated) as percentage of the same subject's 3-day CSP. ns=not significant, **p<0.005.

FIGS. 2A-2E: In vitro Platelet Measurements: Platelets were stored in either plasma alone [Plasma, black circles (10-day storage) and downward triangles (15-day storage)], or in a 65% PAS, 35% plasma mixture (Intersol, black squares, or Isoplate, black, upward triangles) at 4° C., for either 10 or 15 days as indicated. (FIG. 2A) glucose and (FIG. 2B) lactate determined by blood gas analyses, (FIG. 2C) Annexin V positive events, (FIG. 2D) CD62P-positive (P-selectin) events, (FIG. 2E) CD61-positive microparticles, determined by flow cytometry. All results are shown as percentage of the same subject's 3-day 4° C.-stored platelets. *p<0.05, **p<0.01, ***p<0.0001, ns=not significant.

FIGS. 3A, 3B: In Vivo Platelet Measurements: Healthy human subjects received their autologous radiolabeled platelets after storage at 4° C. [Plasma, black circles (10-day storage) and downward triangles (15 day storage)], or in a 65% PAS, 35% plasma mixture (Intersol, black squares, or Isoplate, black, upward triangles). (FIG. 3A) Recovery of transfused platelets at 1 hour time point. (FIG. 3B) Survival of transfused platelets. All results given as percentage of the subject's 3-day autologous radiolabeled 4° C.-stored control platelets. *p<0.05, **p<0.01, ***p=0.001, ns=not significant.

FIG. 4: Platelet-integrin activation: 4° C.-stored platelets were stored for 10 days and either left unstimulated (baseline, left panel) or stimulated with 10 μM adenosine diphosphate (ADP, final concentration) (ADP, right panel). The activation-dependent αIIbβ3-integrin antibody PAC-1 was incubated with both samples along with an activation independent β3-chain antibody (Y-axis, stains all platelets).

FIGS. 5A, 5B: Platelet Yields: [Total platelet yield in the component (platelet count×component volume=platelet yield)] Platelets were stored in plasma for either 5 days at 22° C. (solid black bars), 5 day 4° C.-stored (white with black diagonal stripes), 10 day 4° C.-stored (black with white squares), 15 day 4° C.-stored (black with horizontal white stripes), 20 day 4° C.-stored (gray with vertical white stripes). F=Fresh (pre-storage) sample, S=stored sample. (FIG. 5A) Results are shown as mean±SEM of absolute counts. ns=not significant, ***p<0.001. (FIG. 5B) Results are shown as percentage of the respective fresh sample. ns=not significant, *p≤1.05, n=21 for RT samples, n=5-7 for cold-stored groups.

FIGS. 6A-6E: In Vivo Platelet Characteristics: Healthy human subjects received autologous radiolabeled platelets either fresh (F), or after storage (S) at 4° C. or room temperature (22° C.). Room temperature (black bars), 5-day 4° C.-stored (white with black diagonal stripes), 10 day 4° C.-stored (black with white squares), 15 day 4° C.-stored (black with horizontal white stripes), 20 day 4° C.-stored (gray with vertical white stripes). (FIG. 6A) Recovery of transfused platelets after 2 hours, in fresh (pre-storage) (F) and stored (S) samples shown as mean±SEM. (FIG. 6B) Platelet recovery shown as percentage of the subject's fresh autologous radiolabeled platelets. (FIG. 6C) Survival of transfused platelets of fresh (pre-storage) (F) and stored (S) samples shown as mean±SEM. (FIG. 6D) Survival shown as percentage of the subject's fresh autologous radiolabeled control platelets. (FIG. 6E) Representative traces taken from radiolabeling data (multiple hit model—solid lines, and tangent line—dotted lines). Fresh (gray line with horizontal intersections), 5-day room temperature stored (black line with triangles), 5-day 4° C.-stored (black line with circles), 10 day 4° C.-stored (black line with diamonds), 20 day 4° C.-stored (gray line with squares). *p<0.05, **p<0.01, ***p<0.001, ns=not significant, n=21 for RT and n=5-7 for all other groups.

FIGS. 7A-7D: In Vitro Platelet Metabolism Parameters: Glucose and lactate levels measured by blood gas reader. Room temperature (black bars), 5-day 4° C.-stored (white with black diagonal stripes), 10-day 4° C.-stored (black with white squares), 15-day 4° C.-stored (black with horizontal white stripes), 20-day 4° C.-stored (gray with vertical white stripes). F=Fresh (pre-storage) sample, S=stored sample. (FIG. 7A) Glucose levels of fresh (pre-storage) (F) and stored (S) samples shown as mean±SEM and (FIG. 7B) Glucose levels shown as percentage of corresponding fresh samples, (FIG. 7C) Lactate levels of fresh (pre-storage) (F) and stored (S) samples shown as mean±SEM and (FIG. 7D) Lactate levels shown as percentage of corresponding fresh samples. *p<0.05, **p<0.01, ***p<0.001 comparing stored samples (absolute), or percentage of fresh as indicated, ns=not significant, n=5-7 for all samples.

FIG. 8: Fresh (pre-storage) platelet response for αIIbβ3 integrin activation. Platelet αIIbβ3 integrin activation was measured by PAC-1 antibody binding by flow cytometry. PAC-1 antibody binding was measured at baseline (B) or after stimulation with the agonist collagen (C). PAC-1 binding levels plotted as mean±SEM.

FIGS. 9A-9F: In Vitro Platelet Activation Parameters: Platelet αIIbβ3 integrin activation was measured by PAC-1 antibody binding and α-granule secretion by P-selectin exposure by flow cytometry. PAC-1 antibody binding was measured at baseline (FIG. 9B) or after stimulation with the agonist collagen (FIG. 9C). P-selectin was measured at baseline without agonist. Five-day 4° C.-stored (white with black diagonal stripes), 10 day 4° C.-stored (black with white squares), 15 day 4° C.-stored (black with horizontal white stripes), 20 day 4° C.-stored (gray with vertical white stripes). (FIG. 9A) PAC-1 binding levels shown as mean±SEM and (FIG. 9B) PAC-1 binding after stimulation with collagen as percentage of fresh (pre-storage) sample stimulated with collagen. (FIG. 9C) Representative scatter plots of baseline (pre-storage, no agonist), fresh (pre-storage, stimulated with collagen), 5-day RT (room temperature, stimulated with collagen), 5 day, 15 day, 20 day 4° C.-storage (all stimulated with collagen). (FIG. 9D) Representative histograms of 5-day RT stored platelets stimulated with collagen (dark gray), and 5-day 4° C.-stored platelets (light gray). (FIG. 9E) Percentage P-selectin positive events (CD62P binding) shown as mean and ±SEM. Same groups as outlined above. (F) indicates fresh (pre-storage) sample, and (S) stored sample. (FIG. 9F) P-Selectin exposure as percentage of corresponding fresh samples. *p<0.05, **p<0.01, ***p<0.001, ns=not significant, n=4-8 for all samples.

FIG. 10: Fresh (pre-storage) platelet response for platelet mitochondrial membrane potential. Platelet mitochondrial membrane potential measured as ratio of JC-1 dye red (FL2) to green (FL1) ratio. JC-1 FL2/FL1 ratio was measured by flow cytometry in baseline stored sample (B) and carbonyl cyanide m-chlorophenyl hydrazine (CCCP)-stimulated stored sample (P) and plotted as mean±SEM.

FIGS. 11A-11F: In Vitro Platelet Apoptosis Parameters: Platelet mitochondrial membrane potential measured as JC-1 dye red (FL2) to green (FL-1) ratio. Five-day 4° C.-stored (white with black diagonal stripes), 10-day 4° C.-stored (gray with white squares), 15-day 4° C.-stored (gray with horizontal white stripes), 20 day 4° C.-stored (gray with vertical white stripes). (FIG. 11A) JC-1 FL2/FL1 ratio was measured by flow cytometry in baseline stored sample (B) and carbonyl cyanide m-chlorophenyl hydrazone (CCCP)-stimulated stored sample (P) and shown as mean±SEM, and (FIG. 11B) JC-1 (FL2/FL1) stored baseline samples as percentage of the corresponding fresh baseline samples (pre-storage) shown as mean±SEM. (FIG. 11C) Representative scatter plots of baseline (pre-storage), CCCP (pre-storage, stimulated with CCCP), 5 day RT (room temperature, baseline), 5 day, 15 day, 20 day 4° C.-storage (all baseline). (FIG. 11D) Caspase 3,7 activation measured by flow cytometry and shown as mean fluorescence intensity (MFI) ±SEM. (FIG. 11E) Caspase 3,7 activation shown as percentage of pre-storage sample. *p<0.05, **p<0.01, ***p<0.001, ns=not significant, n=4-8 for all samples. (FIG. 11F) Fresh (pre-storage) platelet response for caspase 3,7 cleavage. Caspase 3,7 cleavage was measured by flow cytometry and plotted as mean fluorescence intensity (MFI)±SEM. Baseline (B), ABT737 (A), n=4-8 for all samples.

FIGS. 12A-12J: Platelet storage temperature and response to agonists for pathways inhibited by DAPT. Human platelets in plasma were obtained by apheresis and either used the same day, or stored at either 4° C., or 22° C. (RT). (FIG. 12A) Platelet αIIbβ3 integrin activation at baseline (gray bar) and after stimulation with 40 μM ADP (black bars), plotted as mean fluorescence intensity (MFI) ±SEM of PAC-1 antibody binding. N=5-13, **p=0.0015 for RT versus fresh, and **p=0.0052 for RT versus 4° C. (FIG. 12B) Representative scatter plots of fresh unstimulated platelets (baseline, BL), and fresh, 4° C., and RT after stimulation with 40 μM ADP. (FIG. 12C) Representative histograms of RT (triangle), 4° C. (star) and fresh (circle) platelets stimulated with 40 μM ADP. (FIG. 12D) Platelet αIIbβ3 integrin activation at baseline (gray bar) and after stimulation with 0.5 mM arachidonic acid (AA, black bars), plotted by mean fluorescence intensity (MFI) of PAC-1 antibody binding. N=5-13, *p=0.0234, (FIG. 12E) Representative scatter plots of fresh unstimulated platelets (baseline, BL), and fresh, 4° C., and RT-stored platelets after stimulation with 0.5 mM arachidonic acid. (FIG. 12F) Representative histograms of RT, 4° C. and fresh platelets stimulated with 0.5 mM arachidonic acid. (FIG. 12G) Dose response studies of platelet aggregation after 5-day 4° C.-storage (squares), or 5-day RT-storage (triangles), or no storage (fresh, circles) stimulated with increasing concentrations of ADP. Plotted as mean percent light transmission ±SEM, N=3-4, *p<0.05, **p<0.01. (FIG. 12H) Representative light transmission aggregometry traces of 5-day 4° C.-stored, 5-day RT-stored or fresh (no storage) platelets after stimulation with 20 μM ADP. (FIG. 12I) Dose response studies of platelet aggregation after 5 day 4° C.-storage (squares), or 5 day RT-storage (triangles), or no storage (fresh, circles) stimulated with increasing concentrations of arachidonic acid. Plotted as mean percent light transmission ±SEM, N=3-5, *p<0.05. (FIG. 12J) Representative light transmission aggregometry traces of 5 day 4° C.-stored, 5 day RT-stored or fresh (no storage) platelets after stimulation with 1 mM arachidonic acid.

FIGS. 13A, 13B: Inhibition of mouse platelets by dual antiplatelet therapy. Mice were treated with a loading dose of acetylsalicylic acid and clopidogrel and platelets were isolated as platelet rich plasma. Shown are representative traces (three separate experiments performed) of aggregation responses after DAPT loading (‘ASA+clopidogrel’) and the aggregation response at baseline (‘control (untreated)’). (FIG. 13A) aggregation response to 0.5 mM arachidonic acid, and (FIG. 13B) aggregation response to 10 μM ADP.

FIGS. 14A-14F: Efficacy of stored platelets to reverse DAPT ex vivo under physiological flow conditions. Platelets in heparinized whole blood from DAPT treated-WT (wild type) mice were labeled with anti-GPIbβ-Alexa488. Fresh or stored platelets (5-day 4° C. (CSP), 5-day RT (RSP)) were labeled with calcein-AM and added to the labeled whole blood at a ratio of 1:2. The mixture was perfused at arterial shear conditions (1500 1/s). (FIG. 14A) The graph shows time traces of the mean fluorescence intensity ±SEM in arbitrary units (A.U., n=6, **p=0.0038) of platelet adhesion and aggregation on fibrillar collagen. (FIG. 14B) Bar graphs show the area coverage by fluorescent platelets after 5 minutes of blood perfusion, expressed as percentage of a pre-defined collagen-coated area. Data are shown as mean±SEM (n=6, *p=0.0302). (FIG. 14C) Representative images of platelet adhesion and aggregation on collagen after 1 min, 3 min, and 5 min of perfusion of the aforementioned groups. (FIG. 14D) The graph shows time traces of the mean fluorescence intensity ±SEM in arbitrary units of platelet adhesion on von Willebrand factor (VWF) (A.U., n=6, n.s.=not significant) (FIG. 14E) Bar graphs show the area coverage by fluorescent platelets after 5 minutes of blood perfusion, expressed as percentage of a pre-defined VWF-coated area. Data are shown as mean±SEM (n=6, n.s.=not significant). (FIG. 14F) Representative images of platelet adhesion on VWF after 1 min, 3 min, and 5 min of perfusion of the aforementioned groups.

FIGS. 15A, 15B: In vivo DAPT reversal in a xeno-transfusion tail bleeding model. When indicated, DAPT-treated NOD/SCID mice received a loading dose of Humate-P 30 min prior to tail cut and transfusion of a human apheresis dose-equivalent 5 min prior and 5 min after tail cut. Blood was collected in Drabkin's reagent. The following groups were used for xenotransfusion: plasma (triangles, volume control), 4° C.-stored (squares), RT-stored (inverted triangles), no DAPT (diamonds, BL), and no transfusion (circles, NT). (FIG. 15A) Hemoglobin concentration in collection fluid over the time course of the experiment plotted as mean±SEM. n=5-8, *p=0.048 for CSP versus RSP. (FIG. 15B) Time to occlusion, n=5-8, *p=0.0383 for CSP versus RSP, *p=0.0493 for plasma control versus 4° C., n.s.=not significant.

FIG. 16: Enrollment and study flow chart. Healthy human subjects were screened, enrolled, and randomized according to the flow diagram. PLT=platelet, CSP=4° C.-stored platelets, RSP=room temperature-stored platelets, HCT=hematocrit, QC=quality control.

FIGS. 17A-17D: Platelet unit and transfusion characteristics. (FIG. 17A) Post storage platelet yield before transfusion. Dot plots for RT-stored (circles), and 4° C.-stored (squares) including bars for mean±SD, n=7-8, n.s.=not significant. (FIG. 17B) Platelet count at different time points of the study, RT-stored (circles), and 4° C.-stored (squares), shown as mean±SEM, n=7-8, *p=0.0110 for 4 h time point and *p=0.0154 for 24 h time point. BL=baseline, no DAPT; LD=loading dose. (FIG. 17C) Corrected count increment at post transfusion time points for RT-stored (triangles) and 4° C.-stored (squares), shown as mean±SEM, n=7-8, *p=0.015 for 4 h time point, and *p=0.0098 for the 24 h time point. (FIG. 17D) Graphical representation of the study arm assignment and length of washout period in between transfusion arms. Left column represents the first (randomized) group assignment (bars indicated by squares: CSP, bars indicated by triangles: RSP), the middle column represents the washout period, and the right column represents the second study assignment (the alternate study product). One subject could not complete the second (RSP) arm because of quality control failure.

FIGS. 18A, 18B: Platelet function characterization by VerifyNOW® (Accumetrics, Inc., San Diego, Calif.). Platelet integrin function was assessed in whole blood by VerifyNOW® point of care test. Shown as RT-stored (circles), and 4° C.-stored (squares). (FIG. 18A) Platelet reactivity tested by VerifyNOW® for acetylsalicylic acid, ARU (aspirin reaction units). (FIG. 18B) Platelet reactivity tested by VerifyNOW® for clopidogrel, PRU (P2Y12 reaction units). Mean±SEM, n=7-8, **p=0.0018. BL=baseline, no DAPT; LD=loading dose.

FIGS. 19A-19C: Platelet function characterization by aggregometry and flow cytometry. (FIG. 19A) Platelets were isolated and washed before assessment by light transmission aggregometry. Traces were analyzed as maximum aggregation (upper row) and area under the curve (lower row). Platelets were stimulated with 0.5 mM arachidonic acid, low dose (LD) ADP (5 μM), high dose (HD) ADP (20 μM), low dose (LD) collagen (2.5 μg/ml), and high dose (HD) collagen (20 μg/ml). (FIG. 19B) Platelet rich plasma was stimulated with arachidonic acid (1 mM), and low dose (LD, 5 μM) and high dose (HD, 20 μM) ADP. (FIG. 19C) Platelet rich plasma was utilized to assess VASP-phosphorylation with a commercially available assay (STAGO, Asnieres sur Seine Cedex, France) by flow cytometry calculated as platelet reactivity index (PRI). The dotted line represents the defined cut off for P2Y12-inhibition at 50%. Data shown as mean±SEM, n=7-8, Maximum aggregation, 1 h: **p=0.0088, AUC, 1 h: *p=0.0214. BL=baseline, no DAPT; LD=loading dose.

FIG. 20. Platelet function characterization by bleeding time (BT). Absolute bleeding time determined as mean of two separate forearm cuts, after transfusion of autologous RT-stored platelets (circles) or autologous 4° C.-stored platelets (squares), shown as mean±SEM, n=7-8.

### DETAILED DESCRIPTION

Platelets play a key role in hemostasis, clot stability and retraction, as well as in vascular repair and anti-microbial host defense. Thrombocytopenia, or low blood platelet count, can result from a number of conditions, which depending on severity, may require the transfusion of donor platelets for treatment. Platelet transfusions are particularly crucial in the treatment of patients with cancer or massive trauma.

Current clinical practice has platelet samples stored at room temperature (RT) (i.e., 20 to 24° C.) after preparation. RT storage maximizes platelet recovery and survival in transfused recipients (Murphy & Gardner. N Engl J Med. 1969; 280(20):1094-1098); however, it also increases the opportunity for bacterial growth in the platelet bag. The FDA limits the shelf life of platelets to 5 days to reduce this bacterial risk. The limited storage time leads to outdates and wastage and subsequent times of shortage especially in rural areas in the US and in far forward military scenarios. Some laboratories can extend the shelf life with an additional bacterial point of care test to 7 days (Li et al. Transfusion. 2017; 57(10):2321-2328). Recently, refined guidelines by the FDA increased the requirement for bacterial testing or alternatively, requested pathogen reduction. These new guidelines will make RT-stored platelets costlier and labor intensive. These recent efforts by the FDA to reduce bacterial contamination highlight the magnitude of the problem for the transfusion medicine community (FDA. Bacterial Risk Control Strategies for Blood Collection Establishments and Transfusion Services to Enhance the Safety and Availability of Platelets for Transfusion). Nevertheless, transfusion associated sepsis from platelet transfusion remains the most common transfusion-transmitted infection and one of the principal lethal risks associated with transfusion (Li et al. Transfusion. 2017; 57(10):2321-2328).

Different technologies have been developed aiming to minimize the risk of bacterial contamination. Examples include diversion pouches for collection, bacterial detection with automatic culture systems and pathogen reduction systems. While these advancements have reduced the number of cases of platelet transfusion associated sepsis, the risk has not been satisfactorily overcome.

Moreover, the current short shelf-life of platelet samples makes it difficult to maintain required inventories. At any given time, up to 20% of platelet samples can be wasted due to expiration. Thus, the extension of platelet sample shelf-life would strengthen the inventory of platelets available for clinical use.

Storage of platelets in cold temperatures would reduce the proliferation of most bacteria and allow a longer period of storage, minimizing shortages currently caused by the short storage time approved by the FDA. Based on this, FDA-approval for the cold-storage of platelets has been sought. Stubbs et al., Transfusion, 57, 2836-2844 (December 2017). However, in the Stubbs et al. study, while various benefits related to cold-storage were achieved, major challenges were noted including a high degree of product wastage. One reason for the high wastage included a short 3 day storage period before product expiration. A second major challenge included macro-aggregate clot formation in 18.2% of samples. Stubbs et al. concluded, “[i]f these limitations could be addressed, [cold-stored platelets] would become a much more practical option for bleeding patients.” Page 2842, 2nd column. Stubbs et al., also stated that cold-stored platelets “with a shelf-life of 10 or more days would allow an expanded inventory of the blood product, and it could very well become the component of choice for all actively bleeding patients.” Page 2843, 1st column.

The current disclosure provides methods for cold-storage of platelet samples with significantly reduced clot formation. In fact, the methods disclosed herein reduced macro-aggregate clot formation from the 18.2% observed in Stubbs et al., Transfusion, 57, 2836-2844 (December 2017) down to less than 3%. This significant decrease allows for better storage of platelet samples, allowing a much-needed expansion of the inventory of this important blood product. Additionally, a cold-stored platelet product produced by the methods of the present disclosure include platelets that: have increased mitochondria preservation; have increased level of pre-activated platelets; and can reverse dual antiplatelet therapy, as measured by decreased blood loss and/or shorter time to occlusion in a xenotransfusion/tail injury mouse model, as compared to a control platelet sample that has been stored at room temperature for 5 days.

In one example of the disclosure, platelets are collected with a TRIMA® (Terumo BCT, Inc., Lakewood, Colo.) apheresis machine (Terumo BCT, Denver Colo.) using the licensed collection kit and extended life platelet (ELP) storage bags (Terumo BCT). The ELP storage bag is a citrated polyvinyl chloride (PVC) FDA-approved for storage of platelets up to seven days post-collection in 100% plasma and up to five days in Isoplate Solution (PAS-F). This is a two-day extension from previous standards for storage of platelets in plasma. For platelet storage up to seven days, the clearance requires that every product must be tested with a bacterial detection device cleared by the FDA and labeled as a “safety measure.” The ELP storage bag can include plasticizers such as tri(2-ethylhexyl) trimellitate (TEHTM or TOTM) or butyryl trihexyl citrate (BTHC). Collections are performed with a yield of 4×10¹¹/bag and a targeted concentration of 1.5×10⁶/μL. Because the actual concentration can differ from the targeted concentration, the concentration can be adjusted with the concurrently collected plasma to 1.5×10⁶/μL. The bag rests at room temperature for 1 hour before being transferred to the cold (4°±2 C). The bag rests at room temperature without agitation and is only removed for sampling purposes. Using this way of storing the platelets, only one unit with a macro-aggregate clot was found 1/51=2%. The n=51 include varying time points between 3 days and 20 days of storage. As indicated, other groups have reported wastage rates of close to 20% with 3 days storage (Stubbs et al., Transfusion, 57, 2836-2844 (December 2017)). Finally, a drop in platelet count was routinely observed, has been reported by other groups before, and likely represents micro-aggregate formation (Getz et al., Transfusion. 2016; 56(6):1320-8; Example 1).

In a second example of the disclosure, prior to apheresis, the pre-apheresis health history questionnaire and check of vital signs are completed. The subject's platelets are collected using the TRIMA ACCEL® (Terumo BCT, Inc., Lakewood, Colo.) Automated Blood Collection System which is licensed by the FDA for this purpose. A venipuncture site is selected and cleaned using standard procedures. A needle is placed in one of the subject's arms at the antecubital area. A complete blood count (CBC) sample is obtained using an inline diversion pouch. Whole blood is drawn into the apheresis machine and the blood components are separated by centrifugation. Platelets and plasma are collected into Terumo ELP storage bags and the red blood cells (RBC) are returned to the subject. Along with the return of the subject's RBC the subject receives 350 mL of ACD (citrate) anticoagulant during the collection process. The platelet apheresis collection lasts 2 hours. Subjects are observed throughout the collection by a nurse or technician specifically trained in apheresis.

A standard single apheresis platelet unit (target platelet yield 3.0×10¹¹/unit and concentration of 1500×10³ platelets/μL) is collected. Fifty milliliters of concurrent plasma is also collected.

Units are calibrated to achieve a final platelet concentration of 0.7-2.1×10⁶ platelets/μL, as per allowable bag parameters.

Immediately after apheresis collection and calibration using sterile techniques, the platelets are left in the attendant plasma. The units rest for 1 hour at room temperature prior to sampling for in vitro assays. Units are weighed to calculate platelet yield. The units are then placed in a locked cage in a refrigerator at 4±2° C. and are not agitated during storage.

Temperature monitors record temperatures and trigger alarms for out of range conditions. End of storage is defined as the date and time when the aliquot is removed from the stored unit for infusion. To date, units have been cold-stored and tested up to 20 days.

Based on the foregoing examples of the disclosure, in particular embodiments, the methods include one or more of the following characteristics: (i) collecting platelets within plasma; (ii) collecting the platelets within a defined yield/bag; (iii) calibrating the concentration of platelets within plasma; (iv) allowing the collected sample to rest at room temperature before cooling; and/or (v) allowing the collected sample to rest at room temperature without agitation before cooling.

In particular embodiments, platelets are collected by apheresis.

In particular embodiments, the defined yield/bag is 2.0×10¹¹-4.5×10¹¹ platelets/bag. In particular embodiments, the defined yield/bag is 3.0×10¹¹-4.0×10¹¹ platelets/bag.

In particular embodiments, the concentration of platelets within plasma is calibrated to 0.5×10⁶-2.3×10⁶ platelets/μL plasma. In particular embodiments, the concentration of platelets within plasma is calibrated to 0.7×10⁶-2.1×10⁶ platelets/μL plasma. In particular embodiments, the concentration of platelets within plasma is calibrated to 1.5×10⁶ platelets/μL plasma.

In particular embodiments, platelet samples remain at room temperature for at least 20 minutes before cooling to 4±2° C. In particular embodiments, platelet samples remain at room temperature for at least 20 minutes, for at least 30 minutes, for at least 40 minutes, for at least 50 minutes, for at least 60 minutes, or more, before cooling to 4±2° C. In particular embodiments, platelet samples remain at room temperature for 20 minutes to 6 hours before cooling to 4±2° C. In particular embodiments, platelet samples remain at room temperature for 20 minutes to 4 hours before cooling to 4±2° C. In particular embodiments, platelet samples remain at room temperature for 20 minutes to 2 hours before cooling to 4±2° C. In particular embodiments, platelet samples remain at room temperature for 1 hour before cooling to 4±2° C.

In particular embodiments, platelet samples remain at room temperature for 6 hours or less before cooling to 4±2° C. In particular embodiments, platelet samples remain at room temperature for 4 hours or less before cooling to 4±2° C. In particular embodiments, platelet samples remain at room temperature for 2 hours or less before cooling to 4±2° C. In particular embodiments, platelet samples remain at room temperature for 1 hour before cooling to 4±2° C.

In particular embodiments, platelet samples remain at room temperature for at least 20 minutes without agitation, for 20 minutes to 6 hours without agitation, for 20 minutes to 4 hours without agitation, for 20 minutes to 2 hours without agitation, or for 1 hour without agitation before cooling to 4±2° C. In particular embodiments, platelet samples remain at room temperature for at least 20 minutes without agitation, for at least 30 minutes without agitation, for at least 40 minutes without agitation, for at least 50 minutes without agitation, for at least 60 minutes without agitation, or more without agitation, before cooling to 4±2° C. In particular embodiments, platelet samples are stored at 4±2° C. without agitation. In particular embodiments, platelet samples remain at room temperature for 6 hours or less without agitation, for 4 hours or less without agitation, for 2 hours or less without agitation, or for 1 hour without agitation before cooling to 4±2° C.

Particular embodiments include (i) collecting platelets collected into plasma utilizing apheresis; (ii) collecting platelets into a bag with a yield of 3.0×10¹¹-4.0×10¹¹ platelets/bag; (iii) calibrating the platelets to a concentration of 0.7×10⁶-2.1×10⁶ platelets/μL plasma; and (iv) allowing the collected and calibrated platelets to rest at room temperature for 1 hour without agitation before being transferred to cold storage at 4±2° C. without agitation.

In particular embodiments, without agitation means that the samples (units) are not manually or mechanically manipulated for the purpose of letting the platelets rest after the apheresis collection which activates platelets due to shear stress. In particular embodiments, without agitation means that the samples are left on a surface free of manual or mechanical manipulation, but for what is required to test the samples for characteristics required for later use. In particular embodiments, without agitation means that the samples are left on a surface free of manual or mechanical manipulation, and without any manipulation or interference during the specified rest period (e.g., 1 hour).

Particular embodiments include populations of cold-stored platelet products produced according to the methods disclosed herein wherein 97% or more of the units within a population remain free of macro-aggregates for at least 10 days after cold storage begins. In particular embodiments, 97% or more of the units within a population remain free of macro-aggregates for at least 20 days after cold storage begins. In particular embodiments, 3% or less of units in a population of 20 units develop macro-aggregates during and/or after cold storage. In particular embodiments, 3% or less of units in a population of 50 units develop macro-aggregates during and/or after cold storage.

Particular embodiments include populations of cold-stored platelet products produced according to the methods disclosed herein wherein 85-100% of the units within a population remain free of macro-aggregates for at least 10 days after cold storage begins. In particular embodiments, 85-100% of the units within a population remain free of macro-aggregates for at least 20 days after cold storage begins. In particular embodiments, 0-15% of units in a population of 20 units develop macro-aggregates during and/or after cold storage. In particular embodiments, 0-15% of units in a population of 50 units develop macro-aggregates during and/or after cold storage.

Particular embodiments include populations of cold-stored platelet products produced according to the methods disclosed herein wherein 85-97% of the units within a population remain free of macro-aggregates for at least 10 days after cold storage begins. In particular embodiments, 85-97% of the units within a population remain free of macro-aggregates for at least 20 days after cold storage begins. In particular embodiments, 3-15% of units in a population of 20 units develop macro-aggregates during and/or after cold storage. In particular embodiments, 3-15% of units in a population of 50 units develop macro-aggregates during and/or after cold storage.

Particular embodiments include populations of cold-stored platelet products produced according to the methods disclosed herein wherein 85%, 86%, 87%, 88%, 89%, 90%, 91%, 92%, 93%, 94%, 95%, 96%, 97%, 98%, 99%, or 100% of the units within a population remain free of macro-aggregates for at least 10 days after cold storage begins. In particular embodiments, 85%, 86%, 87%, 88%, 89%, 90%, 91%, 92%, 93%, 94%, 95%, 96%, 97%, 98%, 99%, or 100% of the units within a population remain free of macro-aggregates for at least 20 days after cold storage begins. In particular embodiments, 0%, 1%, 2%, 3%, 4%, 5%, 6%, 7%, 8%, 9%, 10%, 11%, 12%, 13%, 14%, or 15% of units in a population of 20 units develop macro-aggregates during and/or after cold storage. In particular embodiments, 0%, 1%, 2%, 3%, 4%, 5%, 6%, 7%, 8%, 9%, 10%, 11%, 12%, 13%, 14%, or 15% of units in a population of 50 units develop macro-aggregates during and/or after cold storage.

In particular embodiments, no more than 3 units within a population of cold-stored platelet products include macro-aggregates. In particular embodiments, only 1, 2, or 3 units within a population of cold-stored platelet products include macro-aggregates wherein the population includes at least 20 units.

In particular embodiments, a population of cold-stored platelet products can include 10 units, 20 units, 30 units, 40 units, 50 units, or more.

In particular embodiments, free of macro-aggregates or clot-free means that a cold-stored platelet product lacks visible macro-aggregate formation when visually examined by a blood-banking professional. In particular embodiments utilizing visual inspection, a macro-aggregate is a clot formation that is greater than 5 mm in size and exhibits an “egg drop soup appearance”. Units with such a macro-aggregate clot are not free of macro-aggregates and are not suitable for infusion.

In particular embodiments, free of macro-aggregates or clot-free means that there are no aggregates greater than 2-3 mm by visual inspection, and that all observed aggregates resuspend or are eliminated in a first 200 g spin in a radiolabeling procedure. If aggregates do not resuspend or are not eliminated in the first 200 g spin, the unit is not free of macro-aggregates and is discarded.

Additional aspects and options of the disclosure are now described in the following additional detail: (i) Platelet Sample Collection and Preparation; (ii) Cold Storage of Platelet Samples; (iii) Platelet Products and Transfusion Ready Platelet Products; (iv) Assessment of Platelet Function and/or Characteristics In Vivo and In Vitro; and (v) Control Platelet Samples; and (vi) Methods of Use.

(i) Platelet Sample Collection and Preparation. Withdrawing blood from a donor typically includes inserting a needle into the donor's arm (and, more specifically, the donor's vein) and withdrawing blood from the donor through the needle. The “venipuncture” needle typically has attached to it one end of a plastic tube that provides a flow path for the blood. The other end of the plastic tube terminates in one or more pre-attached plastic blood containers (e.g., bags) for collecting the blood. The needle, tubing and containers make up a blood collection set which is pre-sterilized and disposed of after a single use. Conventional platelet bags or packs are formed of materials that are designed and constructed of a sufficiently permeable material to maximize gas transport into and out of the pack (O₂ in and CO₂ out).

Single donor platelets are platelets obtained from one donor by means of centrifugal separation in an automated apheresis machine in a quantity sufficient to constitute one or more therapeutic dose(s) for subsequent transfusion to a patient(s). Platelets isolated by this method are generally known as single donor platelets because a therapeutic dose can be collected from a single donor. In such a procedure, the donor's blood flows from a point of venipuncture through a sterile centrifuge in which the platelets and a certain volume of plasma are centrifugally separated and isolated, with the balance of the donor's blood being returned to the donor through the initial venipuncture or a second point of venipuncture.

The blood collection container and tubing may include an anticoagulant. Anticoagulants can be used due to the tendency of blood to clot and adhere to the walls of surfaces, such as plastic surfaces. Exemplary anticoagulants are known in the art and include, an anticoagulant citrate phosphate dextrose (CPD) solution, an anticoagulant citrate phosphate double dextrose (CP2D) solution, an anticoagulant citrate phosphate dextrose adenine (CPDA) solution (e.g., CPDA-1), an acid citrate dextrose (ACD) solution (e.g., ACD-A), and an anticoagulant sodium citrate 4% w/v solution.

Various automated apheresis devices are commercially available from companies such as Terumo BCT (Lakewood, Colo.), Haemonetics Corporation (Braintree, Mass.), Fenwal, Inc. (Lake Zurich, Ill.), and Fresenius Kabi (Friedberg, Germany).

The collection of platelets by apheresis generally produces 2 platelet units, wherein each unit contains 200 to 300 mL of plasma and 3.5×10¹¹ platelets. As indicated previously, and in particular embodiments, platelet yields include 2.0×10¹¹ to 4.5×10¹¹ platelets/bag. In particular embodiments, platelet yields include 2.0×10¹¹ platelets/bag, 2.5×10¹¹ platelets/bag, 3.0×10¹¹ platelets/bag, 3.5×10¹¹ platelets/bag, 4.0×10¹¹ platelets/bag, and 4.5×10¹¹ platelets/bag. In particular embodiments, platelet concentrations according to the methods disclosed herein include 0.5×10⁶-2.3×10⁶ platelets/μL plasma and 0.7×10⁶-2.1×10⁶ platelets/μL plasma. In particular embodiments, platelet concentrations can include 0.5×10⁶ platelets/μL plasma, 0.6×10⁶ platelets/μL plasma, 0.7×10⁶ platelets/μL plasma, 0.8×10⁶ platelets/μL plasma, 0.9×10⁶ platelets/μL plasma, 1.0×10⁶ platelets/μL plasma, 1.1×10⁶ platelets/μL plasma, 1.2×10⁶ platelets/μL plasma, 1.3×10⁶ platelets/μL plasma, 1.4×10⁶ platelets/μL plasma, 1.5×10⁶ platelets/μL plasma, 1.6×10⁶ platelets/μL plasma, 1.7×10⁶ platelets/μL plasma, 1.8×10⁶ platelets/μL plasma, 1.9×10⁶ platelets/μL plasma, 2.0×10⁶ platelets/μL plasma, 2.1×10⁶ platelets/μL plasma, 2.2×10⁶ platelets/μL plasma, and 2.3×10⁶ platelets/μL plasma.

While collection of platelets by apheresis is preferred, other methods may also be used. For example, the venipuncture method described above may be used to collect whole blood. It may be desirable for collection of whole blood to be completed relatively quickly, such as for example, with a whole blood donation time of less than 15 minutes. Whole blood collection volumes may vary, for example from 405-495 mL for 450 mL collection containers or from 450-550 mL for 500 mL collection containers. The sterile blood collection container typically serves as the primary container for initial separation of platelets in the buffy coat or platelet rich plasma (PRP) layer.

For preparation of platelets (e.g., whole-blood derived platelets) by the buffy coat method, whole blood is centrifuged under conditions to separate the components into a lower RBC layer, a middle buffy coat layer containing the platelets and an upper platelet poor plasma layer. Buffy coat production methods for platelets are known in the art. Centrifugation conditions may be optimized according to blood center procedures, available centrifugation equipment, etc., but generally the initial centrifugation may be performed, for example, for 7 min. at 5000×g at 22° C., or 5 min. The buffy coat is isolated, for example, by removing (e.g., expressing) the upper plasma layer and the lower red cell layer, leaving the buffy coat in the collection container. The isolated buffy coats may be further processed, for example, by adding plasma, by pooling (e.g., 4-6 buffy coat samples) and/or by adding a platelet additive solution (PAS) to achieve a desired volume and platelet concentration, followed by a lower speed “soft” centrifugation to separate the platelets from white blood cells. Such a lower speed centrifugation may be performed for example, for 3 min. at 2000×g at 22° C. or 8 min. at 500×g at 22° C., followed by expressing the platelet suspension into a storage bag.

For preparation of platelets (e.g., whole blood-derived platelets) by the PRP (platelet rich plasma) method, whole blood is centrifuged under conditions to separate the components into a lower RBC layer containing white blood cells and an upper PRP layer. PRP production methods for platelets are known in the art. Centrifugation conditions may be optimized according to blood center procedures, available centrifugation equipment, etc., but generally the initial centrifugation is performed as a lower speed “soft spin”, for example, for 3 min. at 2000×g at 22° C. PRP is separated from the RBC layer by expressing the upper PRP layer (e.g., using a Compomat G-5), followed by a secondary, faster speed “hard” centrifugation of the PRP to separate the platelets from the plasma. Such a hard spin may be performed for example, for 5 min. at 5000×g at 22° C., followed by removal of plasma from the platelet concentrate, leaving a desired volume of plasma for resuspension of the platelet component. The isolated PRP derived platelet components may be further processed, for example, by adding plasma, pooling (e.g., 4-6 samples) and/or adding a PAS to achieve a desired volume and platelet concentration.

At the time of collection and/or processing, blood may be identified or characterized with respect to one or more parameters, such as for example, hematocrit, hemoglobin, donor gender, whole blood volume, packed cell volume and/or platelet count.

The methods of the current disclosure can be used with platelets isolated by any technique known in the art or developed in the future so long as the percentage of samples exhibiting macro-aggregate formation after 10 days does not exceed 3%.

A platelet sample as disclosed herein includes platelets obtained or prepared by apheresis, buffy coat method, PRP method, or any other platelet preparation method known to one of ordinary skill in the art. In particular embodiments, a platelet sample includes a sample calibrated to contain platelets at a concentration of 0.5×10⁶-2.3×10⁶ platelets/μL, 0.7×10⁶-2.1×10⁶ platelets/μL, or 1.5×10⁶ platelets/μL of plasma. In particular embodiments, a platelet sample includes a sample calibrated to contain platelets at a concentration of 0.5×10⁶-2.3×10⁶ platelets/μL, 0.7×10⁶-2.1×10⁶ platelets/μL, or 1.5×10⁶ platelets/μL of PAS. In particular embodiments, a platelet sample includes a sample calibrated to contain platelets at a concentration of 0.5×10⁶-2.3×10⁶ platelets/μL, 0.7×10⁶-2.1×10⁶ platelets/μL, or 1.5×10⁶ platelets/μL of plasma and/or PAS. In particular embodiments, a platelet sample includes a sample calibrated to contain platelets at a concentration of 0.5×10⁶-2.3×10⁶ platelets/μL, 0.7×10⁶-2.1×10⁶ platelets/μL, or 1.5×10⁶ platelets/μL of 65% PAS/35% plasma. Calibrating a platelet sample can include resuspending the platelet sample after collection in plasma, PAS, or a combination of plasma and PAS to a concentration of 0.5×10⁶-2.3×10⁶ platelets/μL, 0.7×10⁶-2.1×10⁶ platelets/μL, or 1.5×10⁶ platelets/μL. In particular embodiments, a platelet sample can include platelets collected as a platelet unit in a bag at a yield of 2.0×10¹¹-4.5×10¹¹ platelets/bag, or 3.0×10¹¹-4.0×10¹¹ platelets/bag. In particular embodiments, a platelet sample can include platelets pooled from more than one platelet unit. In particular embodiments, a platelet sample can include platelets pooled from more than one individual. In particular embodiments, a platelet sample can include platelets from a single individual.

Before cold storage, a platelet sample can be maintained at room temperature for 6 hours or less, 5 hours or less, 4 hours or less, 3 hours or less, 2 hours or less, 1 hour or less, 30 minutes or less, or 15 minutes or less. In particular embodiments, a platelet sample can be maintained at room temperature for 2 hours or less. In particular embodiments, a platelet sample can be maintained at room temperature for 1 hour. In particular embodiments, the platelet sample can be maintained at room temperature without agitation. Maintaining a platelet sample at room temperature can include storing the platelet sample in an area or location that is at room temperature for a given period of time.

Room temperature in the context of the disclosure can include 20° C. to 24° C. In particular embodiments, room temperature can include 20° C., 21° C., 22° C., 23° C., and 24° C. In particular embodiments, room temperature is 22° C.

(ii) Cold Storage of Platelet Samples. The methods disclosed herein include transferring a platelet sample to cold storage for a period of time after a rest period at room temperature without agitation. Transferring a platelet sample to cold storage includes moving a platelet sample that has been maintained at room temperature to an area or location that is at a cold temperature. Particular embodiments provide for cold storage of a platelet sample at 4±2° C. In particular embodiments, cold storage includes storage at 2° C., 3° C., 4° C., 5° C., or 6° C. In particular embodiments, cold storage includes storage at 4° C. without agitation. Particular embodiments provide for cold storage (4±2° C.) of a platelet sample for at least 24 hours without agitation. In particular embodiments, a platelet sample can be stored in the cold (4±2° C.) for 3 to 20 days without agitation. Particular embodiments provide for cold storage (4±2° C.) of a platelet sample for 3 days, 4 days, 5 days, 6 days, 7 days, 8 days, 9 days, 10 days, 11 days, 12 days, 13 days, 14 days, 15 days, 16 days, 17 days, 18 days, 19 days, and 20 days without agitation. In particular embodiments, a platelet sample can be stored in the cold (4±2° C.) for 10 days or more without agitation. In particular embodiments, a platelet sample can be stored in the cold (4±2° C.) for 20 days or more without agitation. In particular embodiments, platelets can be cold-stored (4±2° C.) in plasma or in an appropriate solution such as PAS, as further described below. In particular embodiments, a platelet sample is clot-free during and/or after it has been stored in the cold (4±2° C.) for at least 24 hours without agitation. In particular embodiments, a platelet sample is clot-free during and/or after it has been stored in the cold (4±2° C.) for 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or more without agitation.

(iii) Platelet Products and Transfusion Ready Platelet Products. The methods disclosed herein are utilized to make platelet samples which include platelet products and transfusion ready platelet products. A population of platelet products include one or more platelet products prepared by the methods disclosed herein.

Platelet products are any blood derived product including platelets as the primary therapeutic component. Platelet products can further include blood plasma, anticoagulant solution used during collection, and alternatively, or in addition, a suitable storage solution, such as a PAS.

Transfusion ready platelet products refer to a platelet product in a storage container (e.g., blood product bag) with suitable labeling for human use, which requires no further processing or treatment of the platelet contents prior to administration to a patient. In particular embodiments, such transfusion ready platelet products include platelets that have been subjected to a pathogen inactivation processing step (e.g., photochemical treatment, such as with a psoralen compound, to inactivate pathogens and leukocytes, if present) during preparation. A transfusion ready platelet product may be produced from an individual platelet unit or more than one unit (e.g., a pooled platelet product) and may be tested for one or more quality measures (e.g., bacterial testing; pH; in vivo platelet recovery post-transfusion; in vivo platelet survival post-transfusion; platelet integrity assessed by, e.g., platelet count, supernatant LDH; platelet apoptosis assessed by, e.g., mitochondrial membrane potential; in vitro platelet metabolism such as glucose concentration and lactate concentration; platelet content; platelet morphology score; and platelet function assessed by, e.g., platelet aggregation or platelet activation; extracellular adenosine triphosphate concentration; total adenosine triphosphate concentration; extent of shape change; and/or platelet hypotonic shock response).

PAS include any suitable aqueous composition that can be used in the storage of a platelet product. Such PAS typically provide nutrients and buffering capacity to allow for extended storage of platelets while maintaining suitable platelet function. PAS typically include sodium chloride and one or more components selected from citrate, phosphate, acetate, magnesium, potassium, calcium, gluconate, glucose, and bicarbonate. The following examples include sodium chloride and the indicated components: PAS-A (also referred to as PAS(1)) further including citrate, phosphate and potassium; PAS-B (also referred to as PAS II, PAS-2, SSP, or T-Sol) further including citrate and acetate: PAS-C (also referred to as PAS III, PAS-3, or Intersol) further including citrate, phosphate, and acetate; PAS-D (also referred to as Composol) further including citrate, phosphate, acetate, magnesium, potassium, and gluconate; PAS-E (also referred to as PAS IIIM or SSP+) further including citrate, phosphate, acetate, magnesium, and potassium; PAS-F (also referred to as PlasmaLyte A) further including acetate, magnesium, potassium, and gluconate; PAS-G further including citrate, phosphate, acetate, magnesium, potassium, and glucose; InterSol-G (also referred to as PAS IV) further including citrate, phosphate, acetate, magnesium, potassium, calcium and glucose; Isoplate (also referred to as Isolyte S) further including phosphate, acetate, magnesium, potassium, and gluconate; PAS V further including citrate, acetate, phosphate, magnesium, potassium, calcium, glucose, and bicarbonate; and M-Sol further including citrate, acetate, magnesium, potassium, calcium, glucose and bicarbonate.

A variety of suitable PAS may be used in the storage of platelets, where such solutions can be added to a unit of platelets in various amounts, such that a unit of platelets may include anywhere from, e.g., 0 to 95% PAS, 5 to 95% PAS, 50 to 95% PAS, 50 to 75% PAS. For example, platelets may be stored in 65% PAS and 35% plasma, providing a unit of platelets including platelets, 65% PAS, and 35% plasma. Typically, in the methods described herein, a unit of platelets will be prepared to a desired level of plasma by addition of the PAS, either automatically in apheresis collection, or manually in the processing of buffy coat or PRP platelets. Platelet additives are described in terms of their aqueous concentration of components prior to their addition to the platelets to give the desired level of plasma in the additive containing unit of platelets.

PAS can include balanced electrolyte solutions with the intent to replace plasma components, including allergens, ABO antibodies, or pathogens (van der Meer & de Korte. Transfus Med Hemother. 2018; 45(2):98-102). In particular embodiments, cold-stored platelets are predominantly intended for actively bleeding surgery and trauma patients, and no hemostatic benefit can be expected from PAS (Capocelli & Dumont. Curr Opin Hematol. 2014; 21(6):491-496). Trauma and surgery patients can require replacement of the entire blood volume by transfusion products, therefore the reduction of plasma in favor of PAS can further dilute coagulation factors possibly to critical levels (Mays & Hess. Blood Transfus. 2017; 15(2):153-157; van Hout et al. Vox Sang. 2017; 112(6):549-556).

(iv) Assessment of Platelet Function and/or Characteristics In Vivo and In Vitro. As indicated, platelet sample function and characteristics can be assessed before and after administration to a patient as a transfusion ready platelet product. Exemplary parameters for testing include platelet count, red and/or white blood cell amount (e.g., contamination), lipid contamination, platelet aggregation, platelet recovery, platelet viability, swirling pattern, potency, platelet survival, morphology, functional activity, activation markers, blood gas (pO₂, pCO₂), pH, glucose, lactate, volume, concentration of growth factors and icterus.

Contamination of platelets may be determined by any of several methods known in the art. For example, contamination of platelets with RBC can be determined by visual inspection for color indicative of RBC contamination. More specifically, RBC contamination above a certain level (e.g., >400 RBC/mL), results in platelets that exhibit discoloration from a light pink/salmon, reddish-orange color tinge to a marked red discoloration, which may be compared to standard visual inspection charts. Lipid contamination (e.g., lipemia) may similarly be determined by visual methods, with increased opacity, ‘milky’ white appearance, large lipid particles that include lipoproteins and chylomicrons, and the like. Platelet aggregation may be determined visually and/or using any of a number of techniques and devices, such as for example, platelet aggregometry, optical aggregometers, lumi-aggregometers, light transmission aggregometry or turbidometric aggregometry. White blood cell contamination may be determined by counting, for example manually performing a leukocyte count (e.g., using a Neubauer counting chamber).

Platelet morphology may be visually inspected at different levels of resolution, including for example, with a discs vs. spheres estimate, and the presence of different morphological forms may be quantitated. In particular embodiments, mean platelet volume (expressed in femtoliters) can be measured to assess platelet size. Platelet volume may be determined by weight and using a factor, such as 1.01 g/ml, as the specific gravity of platelets in PAS. Platelet-derived microparticles can form as a result of platelet activation that occurs during platelet processing and storage. In particular embodiments, microparticles can be quantified by flow cytometric methods based on light-scattering properties.

In particular embodiments, platelet yield of a platelet unit can be calculated by multiplying the platelet count of the platelet unit by the volume of the platelet unit. Platelet counts can be performed by a laboratory machine for the purpose (e.g. ABX Hematology Analyzer from ABX Diagnostics, Irvine, Calif.) or by using a microscope.

Platelet function can be assessed in a number of ways. In particular embodiments, platelet function can be estimated, for example, by platelet response to osmotic stress and by the extent of agonist-induced shape change. In particular embodiments, platelet physiologic response can be evaluated by measuring platelet serotonin uptake and agonist-induced serotonin secretion. Additionally, platelet cellular levels of ATP, glucose, and lactate provide an indication of platelet performance. In particular embodiments, in vitro platelet metabolism can be assessed by measurements of glucose and lactate concentration, and pH at 4° C. with a commercially-available blood gas instrument (e.g., Radiometer Medical, ABL Flex 805, Copenhagen, Denmark).

The quality of a platelet sample can be assessed by in vitro platelet apoptosis assays that can indicate whether platelet mitochondrial membrane is preserved or has been compromised, or whether caspases have been activated. In particular embodiments, mitochondria are preserved when mitochondria membrane potential is maintained or not changed. Changes in the membrane potential can include opening of the mitochondrial permeability transition pore (MPTP), equilibration of ions across the membrane, decoupling of the respiratory chain, and the release of cytochrome c into the cytosol. In particular embodiments, the membrane-permeant JC-1 dye (lipophilic cationic probe 5,5′,6,6′-tetrachloro-1,1′,3,3′-tetraethylbenzimidazolcarbocyanine iodide) can be used to monitor mitochondrial health of platelets (Salvioli et al. FEBS let 1997; 411: 77-82). When the dye is highly concentrated in mitochondria, it forms J-aggregates with an emission maximum at 590 nm (red), while the dye emits at 530 nm (green) when the mitochondria are depolarized. Thus, an increase in mitochondrial preservation in a test platelet sample as compared to the corresponding parameter in an appropriate control platelet sample is indicated by an increase in the red/green fluorescence intensity ratio of JC-1 dye, whereas a decrease in mitochondrial preservation in a test platelet sample as compared to the corresponding parameter in an appropriate control platelet sample is indicated by a decrease in the red/green fluorescence intensity ratio of JC-1 dye. The proton gradient uncoupler carbonyl cyanide m-chlorophenyl hydrazine (CCCP) can be used as a positive control for successful disruption of the electron transport chain. In particular embodiments, caspase 3, 7 activation can be measured using commercially available assays such as CellEvent™ (Life Technologies Corporation, Carlsbad, Calif.) Caspase-3/7 Green Detection Reagent which utilizes a peptide containing a cleavage site for Caspase-3/7 conjugated to a nucleic acid-binding dye with absorption/emission maxima of 502/530 nm (Thermo Fisher Scientific, Waltham, Mass.). The conjugated dye is non-fluorescent until cleaved from the peptide and bound to DNA. After activation of caspase-3/7 in apoptotic cells, the peptide is cleaved, enabling the dye to bind to DNA and produce a bright, fluorogenic response.

The ability of platelets to form clots is an important function of platelets. Activation of platelet αIIbβ3 integrin (gpIIb/IIIa complex) with an agonist such as ADP, arachidonic acid (AA), collagen, or epinephrine, allows the integrin to undergo conformational changes to reveal a ligand binding site for fibrinogen, von Willebrand factor (vWF), fibronectin, and/or vitronectin. This conformational change allows binding of ligand, which is important for platelet aggregation. In particular embodiments, platelet aggregation in a platelet sample indicates integrin activation. In particular embodiments, platelet aggregation in a platelet sample indicates that the platelets are responsive to an agonist of platelet aggregation. In particular embodiments, platelet aggregation can be measured as an increase in light transmission in a platelet sample in response to increasing concentrations of physiologic agonists (such as ADP, collagen, epinephrine), or to dual agonist combinations (e.g., ADP/epinephrine and ADP/collagen) in a αIIbβ3 integrin-dependent manner due to precipitation of platelet aggregates from the platelet sample. In particular embodiments, an increase in pre-activated platelets in a test platelet sample can be indicated by an increase in light transmission through the test platelet sample as compared to the corresponding light transmission in an appropriate control platelet sample, whereas a decrease in pre-activated platelets in a test platelet sample can be indicated by a decrease in light transmission through the test platelet sample as compared to the corresponding light transmission in an appropriate control platelet sample. In particular embodiments, platelet aggregation can be measured as mean percent light transmission ±SEM (standard error of the mean) in a light transmission aggregometry assay. In particular embodiments, ADP can be used as the agonist in platelet activation assays at concentrations from 1 μM to 40 μM, from 10 μM to 40 μM, or from 20 μM to 40 μM. In particular embodiments, ADP can be used as the agonist in platelet activation assays at 10 μM, at 20 μM, or at 40 μM. In particular embodiments, arachidonic acid (AA) can be used as the agonist in platelet activation assays at concentrations from 0.25 mM to 2.5 mM, from 0.5 mM to 2.5 mM, or from 0.5 mM to 2.0 mM. In particular embodiments, AA can be used as the agonist in platelet activation assays at 0.5 mM or at 1 mM.

Assays to assess platelet activation can be performed by flow cytometry as described by Kunicki et al., Transfusion 1975; 15: 414-421. In particular embodiments, expression of the active form of αIIbβ3 integrin can be detected by PAC-1 (clone PAC-1, anti-human CD41/CD61 antibody), a murine monoclonal anti-platelet antibody that binds to activated αIIbβ3 integrin. In particular embodiments, an increase in pre-activated platelets in a test platelet sample can be indicated by an increase in the level of the active form of αIIbβ3 integrin as compared to the corresponding level in an appropriate control platelet sample, whereas a decrease in pre-activated platelets in a test platelet sample can be indicated by a decrease in the level of the active form of αIIbβ3 integrin as compared to the corresponding level in an appropriate control platelet sample. In particular embodiments, an increase in pre-activated platelets in a test platelet sample can be indicated by an increase in the level of anti-human CD41/CD61 antibody binding to platelets in the test sample as compared to the corresponding level in an appropriate control platelet sample, whereas a decrease in pre-activated platelets in a test platelet sample can be indicated by a decrease in the level of anti-human CD41/CD61 antibody binding to platelets in the test sample as compared to the corresponding level in an appropriate control platelet sample. In particular embodiments, the level of anti-human CD41/CD61 antibody binding to platelets in a platelet sample can be measured as mean fluorescence intensity ±standard error of the mean (MFI±SEM) of an anti-human CD41/CD61 antibody conjugated to a fluorescent label.

In particular embodiments, an assay based on the ability of activated αIIbβ3 integrin on platelets to bind fibrinogen-coated microparticles after addition of pathway-specific agonists can be used. In particular embodiments, the assay is based upon a whole blood, point of care test and is commercially available, such as VerifyNOW® (Accumetrics, Inc, San Diego, Calif.). In particular embodiments, a platelet function assay like VerifyNOW® can be used to assess the ability of a platelet sample to reverse the effects of dual antiplatelet therapy (described below).

Activation of platelets is associated with surface expression of various surface antigens, such as for example, GMP-140 (P-selectin, CD62), CD63, and the active form (fibrinogen-binding) of GPIlb/IIIa (αIIbβ3 integrin). In particular embodiments, agonist-induced expression of platelet activation markers such as GMP-140 (P-selectin, CD62) can be measured by flow cytometry. Antibody binding of P-selectin can indicate α-granule secretion from platelets. In particular embodiments, platelet activation can be assessed by markers indicating membrane orientation changes with phosphatidyl serine exposure, such as Annexin V binding to platelets. Thromboglobulin and/or Platelet Factor 4 released by activated platelets into the medium are platelet-specific proteins and can be measured as indicators of platelet activation. Platelet Factor 3 activity (procoagulant surface for binding clotting proteins) also increases with platelet activation. Assays are also commercially available to perform such characterization of platelets in many cases.

In particular embodiments, in vivo platelet viability can be assessed by measuring in vivo platelet recovery post-transfusion. After collection and preparation of a platelet sample from a subject, the platelet sample can be stored for a given period of time at a given temperature before being (radio)labeled. The stored platelet sample can be referred to as a test platelet sample. Alternatively, a platelet sample can be (radio)labeled after collection without being stored (if the platelet sample is to serve as a fresh control) and can be referred to as a control platelet sample. The platelet sample can be radiolabeled as described in the Biomedical Excellence for Safer Transfusion (BEST) collaborative protocol. (BEST)Collaborative. TBEfST. Platelet radiolabeling procedure. Transfusion 2006; 46:59S-66S. In particular embodiments, appropriate radioisotopes for labeling include Indium 111 and Chromium 51. (Radio)labeled platelet samples can be autologously transfused into a subject. Follow-up samples from the subject can be collected 2 hours post-transfusion and on day 1, day 2, day 3, day 4, day 5, day 6, and day 7 post-transfusion to calculate recovery and survival of a subject's platelets. In particular embodiments, two follow-up platelet samples can be collected during day 1 post-transfusion, two follow-up platelet samples can be collected during day 2 post-transfusion, or two follow-up platelet samples can be collected during day 1 post-transfusion and two follow-up platelet samples can be collected during day 2 post-transfusion. In particular embodiments, if two follow-up platelet samples are taken on the same day, the samples can be obtained 2 to 10 hours apart. In particular embodiments, one follow-up platelet sample can be collected during each of day 3, day 4, day 5, day 6, and day 7 post-transfusion. In particular embodiments, platelet survival can be assessed by measuring the disappearance rate of the (radio)labeled platelets from circulation in a subject. In particular embodiments, platelet survival of a platelet sample can be expressed in hours or days. In particular embodiments, platelet survival of a platelet sample can be expressed as a percent of platelet survival of a control (radio)labeled platelet sample. In particular embodiments, mean survival time of platelets is derived from PRP that had been stored for 5 days, 10 days, 15 days, or 20 days at 4±2° C. as compared to the mean survival time of fresh platelets from the same subject, measured in days. In particular embodiments, platelet recovery can be assessed by measuring the amount of (radio)labeled platelets recovered from a subject at a given time post-transfusion. In particular embodiments, platelet recovery of a test platelet sample can be expressed as a percent of platelet recovery of a control (radio)labeled platelet sample. In particular embodiments, platelet recovery is derived from PRP that had been stored for 5 days, 10 days, 15 days, or 20 days at 4±2° C., measured as a percentage of platelets derived from freshly collected PRP from the same subject, utilizing the 2-hour post-transfusion time point. In particular embodiments, the control (radio)labeled platelet sample is a fresh platelet sample that is collected from a subject and transfused into the same subject the same day. In particular embodiments, the control (radio)labeled platelet sample is collected from a subject and transfused into the same subject 7 to 14 days after transfusion of a test platelet sample.

Platelet function can also be assessed after transfusion in the context of reversing dual antiplatelet therapy (DAPT). DAPT is performed routinely for the prevention of early and late stent thromboses after coronary intervention in cardiac patients. DAPT comprises drug-induced platelet function impairment by acetylsalicylic acid (ASA or aspirin) in combination with P2Y12 inhibitors, like clopidogrel. P2Y12 inhibitors (receptor blockers) are a group of antiplatelet drugs. This group of drugs includes: clopidogrel, ticlopidine, ticagrelor, prasugrel, and cangrelor. Endogenous platelets of subjects can be inhibited with DAPT and cold-stored platelets (or appropriate platelet sample control) can be transfused into DAPT subjects. The function of cold-stored platelets (or appropriate platelet sample control) can be assessed in vivo and ex vivo before transfusion using assays as described herein. The ability of transfused platelets to reverse DAPT can be measured in vivo and in vitro after transfusion using assays as described herein.

A flow chamber assay can be used to measure ability of platelets in a test platelet sample to reverse DAPT ex vivo under physiological flow conditions. The assay measures platelet adhesion and aggregation by formation of three-dimensional thrombi after perfusing a mixed platelet population (exogenous test platelet sample mixed with endogenous labeled DAPT platelets in a particular ratio, e.g., 1:3) at physiological shear rate over fibrillar collagen in a microfluidic device or flow chamber. In particular embodiments, an ability of platelets in a test platelet sample to reverse DAPT ex vivo can be indicated by more or larger thrombi formation as compared to platelets in an appropriate control platelet sample. In particular embodiments, thrombi formation with fluorescent platelets can be measured by mean fluorescence intensity and area coverage of the fluorescent platelets can be expressed as percentage of a pre-defined collagen-coated area. In particular embodiments, the mixed platelet populations are perfused for 1 min, 2 min, 3 min, 4 min, 5 min, or longer. In particular embodiments, another adhesive protein instead of collagen can be used, such as von Willebrand factor (VWF). In particular embodiments, an anti-GPIbβ antibody conjugated to a fluorescent label can be used to label the endogenouse DAPT platelets.

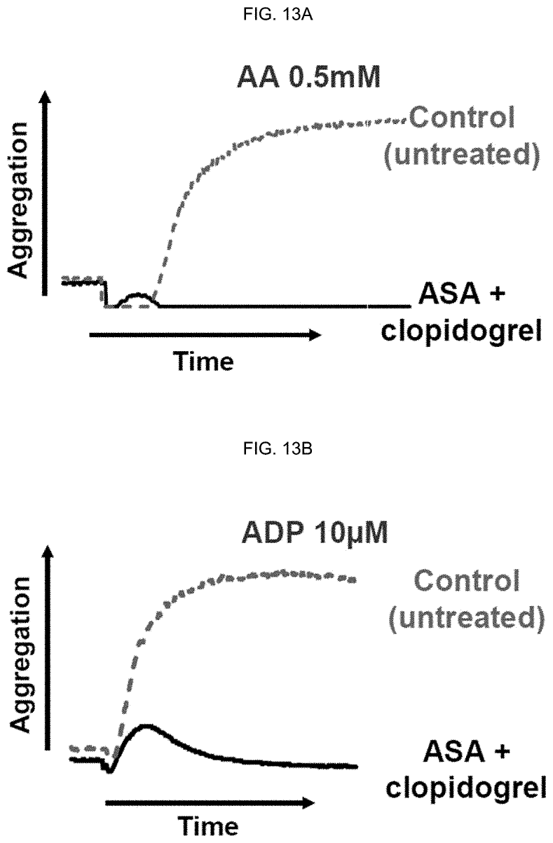

A hybrid xenotransfusion/tail injury mouse model based on a previously described model (Saito et al. International journal of experimental pathology. 2016; 97(3):285-292) can be used to measure ability of a test platelet sample to reverse DAPT in vivo. In this model, NOD/SCID mice are treated with DAPT and inhibition of platelet responses to ADP and arachidonic acid can be verified by aggregometry similar to experiments described in FIGS. 13A and 13B. A test platelet sample can be transfused 5 min prior and 5 min after tail cut and blood can be collected over 30 min until the experiment is terminated. Blood loss from the mice (measured by hemoglobin concentration in collection fluid over time) and time to occlusion (defined as the earliest time point when no change in hemoglobin concentration is recorded compared to later time points) can be measured. In particular embodiments, human VWF (e.g., Humate-P) at concentrations equivalent to therapeutic doses given to humans with active bleeding is perfused into mice 30 min prior to the experiment to promote human platelet function with human VWF at the site of injury. In particular embodiments, an ability of platelets in a test platelet sample to reverse DAPT in vivo can be indicated by less blood loss in a xenotransfusion/tail injury mouse model as compared to platelets in an appropriate control platelet sample. In particular embodiments, an ability of platelets in a test platelet sample to reverse DAPT in vivo can be indicated by shorter time to occlusion in a xenotransfusion/tail injury mouse model as compared to platelets in an appropriate control platelet sample.

In particular embodiments, a bleeding time assay can be used to measure in vivo function of platelets in a platelet sample. The assay can be performed as described in Slichter et al. Blood. 2015; 126. A sphygmomanometer can be inflated to 40 mmHg on the left or right arm to allow arterial inflow, but block venous outflow. A sterile assay template with an attached sterile blade can be used to perform three scratches on the corresponding forearm with 1 mm and 9 mm length on the volar side. The time required for cessation of bleeding can then be measured by blotting with filter paper every, e.g., 30 seconds, without disturbing the wound. The test can be stopped after, e.g., 30 min, and steri-strips can be applied perpendicularly to the incision site to avoid scarring.

In particular embodiments, phosphorylation of VASP (vasodilator-stimulated phosphoprotein), an intraplatelet actin regulatory protein, can be used to measure ability of a test platelet sample to reverse DAPT in vitro. Phosphorylation of VASP is dependent on the level of activation of the platelet P2Y12 receptor, which is targeted by inhibitors such as clopidogrel. A flow cytometric assay can be used, and a platelet reactivity index (PRI) of VASP can be calculated. PRI, expressed as a percentage, is the difference in VASP fluorescence intensity between resting and activated platelets.

(v) Control Platelet Samples. A control platelet sample can include a fresh platelet sample, or a platelet sample that has been stored for a given amount of time at a given temperature. In particular embodiments, a fresh control platelet sample has not been stored. In particular embodiments, a control platelet sample has been stored at 4±2° C. In particular embodiments, a control platelet sample has been stored at 4±2° C. for 3 days. In particular embodiments, a control platelet sample has been stored at room temperature (20-24° C.). In particular embodiments, a control platelet sample has been stored at room temperature (20-24° C.) for 5 days. In particular embodiments, a control platelet sample can be from a single individual or from a pool of individuals. In particular embodiments, a control platelet sample can be autologous, or originating from the same individual who is receiving an infusion of cold-stored platelets. In particular embodiments, a control platelet sample can include a platelet sample derived from a healthy individual or an individual with no known bleeding disorders.

(vi) Methods of Use. The cold-stored platelet products disclosed herein can be used for treating subjects. Subjects include humans, mice, dogs, cats, reptiles, birds, horses, cattle, goats, pigs, chickens, monkeys, rats, etc. Treating subjects includes delivering therapeutically effective amounts. Therapeutically effective amounts include those that provide effective amounts, prophylactic treatments, and/or therapeutic treatments.

An “effective amount” is the amount of a cold-stored platelet product necessary to result in a desired physiological change in a subject. Effective amounts are often administered for research purposes. In particular embodiments, an effective amount of a cold-stored platelet product can treat bleeding in subjects with low plate count and/or platelet dysfunction. In particular embodiments, an effective amount of a cold-stored platelet product can treat bleeding in subjects with cancer.

A “prophylactic treatment” includes a treatment administered to a subject who does not display signs or symptoms of a condition to be treated or displays only early signs or symptoms of the condition to be treated such that treatment is administered for the purpose of diminishing, preventing, or decreasing the risk of developing the condition. Thus, a prophylactic treatment functions as a preventative treatment against a condition. In particular embodiments, a prophylactic treatment is administered to a subject who is not actively bleeding. In particular embodiments, a prophylactic treatment is administered to a subject with hypoproliferative thrombocytopenia. In particular embodiments, a prophylactic treatment is administered to a subject who exhibits a platelet count below 50×10⁶ platelets/μL, below 40×10⁶ platelets/μL, below 30×10⁶ platelets/μL, below 20×10⁶ platelets/μL, below 10×10⁶ platelets/μL, or below 5×10⁶ platelets/μL.

A “therapeutic treatment” includes a treatment administered to a subject who displays symptoms or signs of a condition and is administered to the subject for the purpose of reducing the severity or progression of the condition. In particular embodiments, a therapeutic treatment is administered to a trauma subject or subject undergoing surgery who is actively bleeding. In particular embodiments, a therapeutic treatment is administered to a subject who exhibits a platelet count of 5,000 to 20,000 platelets/μL.

The actual dose and amount of a cold-stored platelet product administered to a particular subject can be determined by a physician, veterinarian, or researcher taking into account parameters such as physical and physiological factors including target; body weight; type of condition; severity of condition; upcoming relevant events, when known; previous or concurrent therapeutic interventions; and idiopathy of the subject, for example. In addition, in vitro and in vivo assays as described herein can optionally be employed to help identify optimal dosage ranges.

Therapeutically effective amounts of a cold-stored platelet product can include doses ranging from, for example, 1×10¹¹ to 5×10¹¹ platelets/m² or from 2×10¹¹ to 5×10¹¹ platelets/m². In other examples, a dose can include 1×10¹¹ platelets/m², 1.5×10¹¹ platelets/m², 2×10¹¹ platelets/m², 2.5×10¹¹ platelets/m², 3×10¹¹ platelets/m², 3.5×10¹¹ platelets/m², 4×10¹¹ platelets/m², 4.5×10¹¹ platelets/m², 5×10¹¹ platelets/m², or more. In particular embodiments, a therapeutically effective amount of a cold-stored platelet product includes 3×10¹¹ to 4×10¹¹ platelets/m². In particular embodiments, a therapeutically effective amount of a cold-stored platelet product can be administered intravenously. In particular embodiments, the cold-stored platelet product has been stored at 4±2° C. for 5 days, 10 days, 15 days, 20 days, or more.

In particular embodiments, the effectiveness of a cold-stored platelet product post-transfusion can be assessed by ex vivo and in vivo assays described herein.

The Exemplary Embodiments and Examples below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.

### EXEMPLARY EMBODIMENTS

1. A method of forming a cold-stored platelet product including:
collecting platelet and plasma from a subject;
calibrating the platelets to a concentration of 0.5×10⁶-2.3×10⁶ platelets/μL of the plasma and/or a platelet additive solution (PAS) to create a platelet sample;
maintaining the platelet sample at room temperature for 20 minutes to 6 hours, for 20 minutes to 4 hours, or for 20 minutes to 2 hours; and
transferring the platelet sample to cold-storage for a period of time, thereby forming a cold-stored platelet product.
2. A method of embodiment 1, wherein the collecting includes by apheresis, buffy coat method, or platelet rich product (PRP) method.
3. A method of embodiment 1 or 2, wherein the collecting is by apheresis.
4. A method of any of embodiments 1-3, wherein the collecting of platelets is to a yield of 2.0×10¹¹-4.5×10¹¹ platelets/bag.
5. A method of any of embodiments 1-4, wherein the collecting of platelets is to a yield of 3.0×10¹¹-4.0×10¹¹ platelets/bag.
6. A method of any of embodiments 1-5, wherein the concentration is 0.7×10⁶-2.1×10⁶ platelets/μL of the plasma.
7. A method of any of embodiments 1-6, wherein the concentration is 1.5×10⁶ platelets/μL of the plasma.
8. A method of any of embodiments 1-5, wherein the concentration is 0.7×10⁶-2.1×10⁶ platelets/μL of the PAS.
9. A method of any of embodiments 1-6, wherein the concentration is 1.5×10⁶ platelets/μL of the PAS.
10. A method of any of embodiments 1-9, wherein the concentration is 0.7×10⁶-2.1×10⁶ platelets/μL of the plasma and PAS.
11. A method of any of embodiments 1-10, wherein the concentration is 1.5×10⁶ platelets/μL of the plasma and PAS.
12. A method of any of embodiments 1-11, wherein the percentage of PAS is 65% and the percentage of plasma is 35%.
13. A method of any of embodiments 1-12, wherein the maintaining the platelet sample at room temperature is for 20 minutes to 2 hours.
14. A method of any of embodiments 1-13, wherein the maintaining the platelet sample at room temperature is for 1 hour.
15. A method of any of embodiments 1-14, wherein the maintaining the platelet sample at room temperature is without agitation of the platelet sample.
16. A method of any of embodiments 1-15, wherein room temperature is 20-24° C.
17. A method of any of embodiments 1-16, wherein the cold storage is 4±2° C.
18. A method of any of embodiments 1-16, wherein the cold storage is 4° C.
19. A method of any of embodiments 1-18, wherein the period of time is at least 24 hours.
20. A method of any of embodiments 1-18, wherein the period of time is 3-20 days.
21. A method of any of embodiments 1-18, wherein the period of time is 10 days or more.
22. A method of any of embodiments 1-21, wherein the platelet sample is clot-free during and/or after the period of time.
23. A method of any of embodiments 1-22, wherein the platelet sample is clot-free during and/or after 3 to 20 days of cold storage.
24. A method of any of embodiments 1-23, wherein the platelet sample is clot-free during and/or after 5 days of cold storage.
25. A method of any of embodiments 1-24, wherein the platelet sample is clot-free during and/or after 20 days of cold storage.
26. A method of any of embodiments 1-25, wherein the cold-stored platelet product is free of macro-aggregates for up to 20 days at cold storage.
27. A method of any of embodiments 1-26, wherein the platelet product is a transfusion ready platelet product.
28. A population of cold-stored platelet products produced according to any of the methods of embodiments 1-27 wherein each cold-stored platelet product is a unit and wherein 97% or more of the units within the population remain free of macro-aggregates for at least 10 days after cold storage begins.
29. A population of embodiment 28, wherein 97% or more of the units within the population remain free of macro-aggregates for at least 20 days after cold storage begins.
30. A population of cold-stored platelet products produced according to any of the methods of embodiments 1-27 wherein each cold-stored platelet product is a unit and wherein 85%-97% of the units within the population remain free of macro-aggregates for at least 10 days after cold storage begins.
31. A population of embodiment 30, wherein 85%-97% of the units within the population remain free of macro-aggregates for at least 20 days after cold storage begins.
32. A population of cold-stored platelet products produced according to any of the methods of embodiments 1-27 wherein each cold-stored platelet product is a unit and wherein 85%, 86%, 87%, 88%, 89%, 90%, 91%, 92%, 93%, 94%, 95%, 96%, 97%, 98%, 99%, or 100% of the units within the population remain free of macro-aggregates for at least 10 days after cold storage begins.
33. A population of embodiment 32, wherein 85%, 86%, 87%, 88%, 89%, 90%, 91%, 92%, 93%, 94%, 95%, 96%, 97%, 98%, 99%, or 100% of the units within the population remain free of macro-aggregates for at least 20 days after cold storage begins.
34. A population of any of embodiments 27-33, wherein the platelet products include transfusion ready platelet products.
35. A population of cold-stored platelet products, wherein each cold-stored platelet product is prepared by a process including:
   collecting platelets and plasma from a subject;
   calibrating the platelets to a concentration of 0.5×10⁶-2.3×10⁶ platelets/μL of the plasma and/or a platelet additive solution (PAS) to create a platelet sample;
   maintaining the platelet sample at room temperature for 20 minutes to 6 hours, for 20 minutes to 4 hours, or for 20 minutes to 2 hours; and
   transferring the platelet sample to cold-storage for a period of time.
36. A population of embodiment 35, wherein each cold-stored platelet product is a unit and wherein 97% or more of the population remains free of macro-aggregates for at least 10 days after cold storage begins.
37. A population of embodiment 35 or 36, wherein each cold-stored platelet product is a unit and wherein 97% or more of the population remains free of macro-aggregates for at least 20 days after cold storage begins.
38. A population of any one of embodiments 35-37, wherein each cold-stored platelet product is a unit and wherein 3% or less of units in a population of 20 units develop macro-aggregates after 10 days of cold storage.
39. A population of any one of embodiments 35-38, wherein each cold-stored platelet product is a unit and wherein 3% or less of units in a population of 20 units develop macro-aggregates after 20 days of cold storage.
40. A population of any one of embodiments 35-37, wherein each cold-stored platelet product is a unit and wherein 3% or less of units in a population of 50 units develop macro-aggregates after 10 days of cold storage.
41. A population of any one of embodiments 35-37, or 40, wherein each cold-stored platelet product is a unit and wherein 3% or less of units in a population of 50 units develop macro-aggregates after 20 days of cold storage.
42. A population of any one of embodiments 35-41, wherein the platelet products are transfusion ready platelet products.
43. A cold-stored platelet product prepared by a process including:
   collecting platelets and plasma from a subject;
   calibrating the platelets to a concentration of 0.5×10⁶-2.3×10⁶ platelets/μL of the plasma and/or a platelet additive solution (PAS) to create a platelet sample;
   maintaining the platelet sample at room temperature for 20 minutes to 6 hours, for 20 minutes to 4 hours, or for 20 minutes to 2 hours; and
   transferring the platelet sample to cold-storage for a period of time.
44. A cold-stored platelet product of embodiment 43, wherein the product remains free of macro-aggregates for at least 5 days after cold storage begins.
45. A cold-stored platelet product of embodiment 43 or 44, wherein the product remains free of macro-aggregates for at least 10 days after cold storage begins.
46. A cold-stored platelet product of any one of embodiments 43-45, wherein the product remains free of macro-aggregates for at least 20 days after cold storage begins.
47. A cold-stored platelet product of any one of embodiments 43-46, wherein the platelet product is a transfusion ready platelet product.
48. A cold-stored platelet product of any one of embodiments 43-47, wherein the cold-stored platelet product includes platelets with an increase in mitochondria preservation as compared to platelets in a control platelet sample.
49. A cold-stored platelet product of embodiment 48, wherein mitochondria preservation is measured by an assay including lipophilic cationic probe 5,5′,6,6′-tetrachloro-1,1′,3,3′-tetraethylbenzimidazolcarbocyanine iodide.
50. A cold-stored platelet product of any one of embodiments 43-49, wherein the cold-stored platelet product includes platelets with an increase in pre-activated platelets as compared to platelets in a control platelet sample.
51. A cold-stored platelet product of embodiment 50, wherein a level of pre-activated platelets is measured by a light transmission aggregometry assay.
52. A cold-stored platelet product of embodiment 50 or 51, wherein a level of pre-activated platelets is measured by an assay including an anti-human CD41/CD61 antibody.
53. A cold-stored platelet product of any one of embodiments 50-52, wherein a level of pre-activated platelets is measured by a whole blood, point of care assay including reagents to measure the ability of αIIbβ3 integrin on platelets to bind fibrinogen.
54. A cold-stored platelet product of any one of embodiments 43-53, wherein the platelet product reverses dual antiplatelet therapy (DAPT) ex vivo as compared to a control platelet sample.
55. A cold-stored platelet product of embodiment 54, wherein reversal of DAPT ex vivo is measured by platelet adhesion and aggregation in a flow chamber.
56. A cold-stored platelet product of any one of embodiments 43-53, wherein the platelet product reverses dual antiplatelet therapy (DAPT) in vivo as compared to a control platelet sample.
57. A cold-stored platelet product of embodiment 56, wherein reversal of DAPT in vivo is measured by blood loss in a xenotransfusion/tail injury mouse model.
58. A cold-stored platelet product of embodiment 56 or 57, wherein reversal of DAPT in vivo is measured by time to occlusion in a xenotransfusion/tail injury mouse model.
59. A cold-stored platelet product of any one of embodiments 48-58, wherein the control platelet sample is a platelet sample stored at room temperature for 5 days.
60. A method of treating a subject in need of a platelet transfusion including administering to the subject a therapeutically effective amount of a cold-stored platelet product of any of embodiments 43-59.
61. A method of embodiment 60, wherein the subject has a low platelet count and/or platelet dysfunction.
62. A method of embodiment 60 or 61, wherein the subject has hypoproliferative thrombocytopenia.
63. A method of any one of embodiments 60-62, wherein the subject is not actively bleeding.
64. A method of any one of embodiments 60-62, wherein the subject is actively bleeding.
65. A method of any one of embodiments 60-64, wherein the subject is undergoing surgery.
66. A method of any one of embodiments 60-65, wherein the subject has cancer.
67. A method of any one of embodiments 60-66, wherein the administering is intravenously.

### Example 1

In vivo Viability of Extended 4° C. Stored Autologous Apheresis Platelets. Introduction. Most U.S. Blood Banks store platelets at room temperature (RT) under gentle agitation with a maximum storage time of 5 days unless additional in vitro bacterial testing is done which permits 7 day storage. Previous studies demonstrated that storing platelets in the cold (4° C., CSP) resulted in a significant reduction in both in vivo recoveries and survivals compared with RT platelets (RSP) stored for the same times. Murphy & Gardner, N Engl J Med 1969; 280: 1094-1098. Subsequent studies with cold-stored platelets identified a clearance mechanism involving GPIb clustering on the platelet surface and in vivo binding to complement type 3 receptors with subsequent hepatocyte internalization. Hoffmeister et al., Cell 2003; 112: 87-97; Snyder & Rinder, N Engl J Med 2003; 348: 2032-2033. In vitro studies suggest that 4° C.-stored platelets have superior functionality compared with RT-stored platelets. Bynum et al., Transfusion 2016; 56 Suppl 1: S76-S84; Getz et al., Transfusion 2016; 56: 1320-1328; Nair et al., Br J Haematol 2017; 178: 119-129; Reddoch et al., Shock 2014; 41 Suppl 1:54-61; Becker et al., Transfusion 1973; 13: 61-68. Furthermore, storage of platelets in the cold (4° C.) has the advantage of potentially prolonging storage times while reducing post-transfusion infections. RT storage has led to a 5 day storage limit since bacterial growth and septic reactions increase over time. Braine et al., Transfusion 1986; 26: 391-393. This limited shelf-life leads to periodic shortages on the one hand and frequent outdates on the other. Contrary to red cells, platelet usage is still slightly increasing according to recently published data. Braine et al., Transfusion 1986; 26: 391-393. Having an additional platelet inventory with extended storage in the cold for specifically targeted patient populations could lead to both greater platelet availability and fewer outdates.

When first introduced, platelet transfusions were predominantly used for the prophylactic transfusion support of hematology/oncology patients with hypoproliferative thrombocytopenia. These patients benefit from prolonged post-transfusion platelet survivals to decrease transfusion frequency. However, a recent analysis showed that as many as 50% of the platelets transfused are given to non-hematology/oncology patients (i.e., trauma, surgery, ICU, and general medicine patients) who often require only short-term hemostatic support. Braine et al., Transfusion 1986; 26: 391-393.

Two randomized, controlled clinical trials are available suggesting that CSP are more hemostatically active than RSP. A trial in pediatric cardiac surgery patients showed that whole blood stored in the cold (4° C.) was more effective in reducing blood loss during open heart surgery compared with reconstituted whole blood containing RSP (22° C.). Interestingly, patients who received cold-stored whole blood had significantly better aggregometry responses than RSP-containing reconstituted whole blood. Manno et al., Blood 1991; 77: 930-936. Preliminary analysis of an ongoing Norwegian randomized, pilot-trial in open-heart surgery patients showed that patients who received up to 7 day stored CSP in a platelet additive solution (T-PAS, Terumo BCT, Denver, Colo.) had significantly reduced post-operative blood loss with CSP compared with RSP. Manno et al., Blood 1991; 77: 930-936.

Currently, the FDA allows transfusion of 3 day CSP (whole-blood derived pooled platelets or apheresis platelets) for actively bleeding patients. Cap, Transfusion 2016; 56: 13-16. CSP have not been widely utilized presumably due to a lack of data suggesting superior efficacy in the target population of bleeding patients. Furthermore, the 3 day storage limit is even shorter than the limit for RSP, thus offering even less flexibility.

Platelet storage in PAS has been shown to allow for prolongation of the storage time in RT platelet storage studies. Slichter et al., Blood 2014; 123: 271-280. Currently, only two PAS are licensed by the FDA for RT storage, and they have not been widely adopted. Getz et al., Transfusion 2016; 56: 1320-1328; Manno et al., Blood 1991; 77: 930-936; Slichter et al., Blood 2014; 123: 271-280. Before the current study, it was unknown how PAS may affect in vivo viability of platelets stored at 4° C. In the current study, the in vivo viability of autologous radiolabeled extended cold stored platelets in plasma versus PAS in normal subjects along with in vitro tests currently required by the FDA for platelet licensing were investigated.

Materials & Methods. Preparation of Control and Test platelets. A double hyperconcentrated apheresis platelet unit was collected from 20 healthy adult subjects using the TRIMA ACCEL® Automated Blood Collection System (TerumoBCT, Denver, Colo.). After collection, the unit was split into two equal portions, and one unit was re-suspended in 100% plasma for 3 day 4° C. storage (control unit was stored for the longest FDA licensed time), and the other unit was stored for an extended period of time at 4° C. (test unit). The test unit was suspended in 100% plasma or 35% plasma and 65% PAS (either Isoplate [PAS-F, TerumoBCT, Denver, Colo.] or Intersol [PAS-3, Fenwal Inc., Lake Zurich, Ill.]). Both control and test units had to achieve a final platelet concentration of 1500×10³ platelets/μL and they were stored, without agitation, at 4° C. The test units were stored for up to 10 days (plasma or plasma/PAS) or 15 days (plasma).

Radiolabeling of stored platelets. Nineteen out of 20 subjects were available for in vivo assessment. One subject did not show for autologous transfusion. Platelets were radiolabeled as previously described, following the detailed Biomedical Excellence for Safer Transfusion (BEST) collaborative protocol. (BEST)Collaborative. TBEfST. Platelet radiolabeling procedure. Transfusion 2006; 46:59S-66S. In brief, Indium (In-111, Anazao, Tampa, Fla., USA) was used to label both control and test platelets because according to earlier experiments (data not shown), the other available isotope, chromium, demonstrated very poor uptake of Cr-51 by refrigerated platelets. The In-111 administered on Day 3 CSP was largely undetectable by Day 10 and therefore, re-use of the same isotope to measure both control and test cold stored platelets was considered feasible. Additionally, a pre-infusion radioactivity sample was collected prior to the “test” transfusion to account for any possible residual In-111 activity after each subject's control transfusion. Calculations were adjusted accordingly.

On Day 3, the subject received an In-111 radiolabeled aliquot of their control CSP stored platelet unit. Follow-up samples from the subject were collected 2 hours post-infusion and on Days 1 (×2, 2-10 hours apart), 2, 3, 4, and 5 to calculate recovery and survival of the subject's 3 day stored platelets.

The CSP test platelets in 100% plasma or PAS/plasma were initially stored for 10 days, and another group of subjects had their platelets stored in 100% plasma for 15 days. After storage of their test units, an aliquot of their test units was obtained for In-111 labeling and subsequently transfused. Follow-up samples, as above, were collected to calculate platelet recovery and survival of the test platelets. These studies allowed direct comparisons of the same subject's control 3-day CSP versus 10- or 15 day test stored CSP. Comparisons were further facilitated as both control and test platelets were labeled with the same isotope.

In vitro platelet measurements. Platelet counts of collected products were performed on the day following collection to allow platelet disaggregation that might have occurred during collection and at the end of storage using an ABX Hematology Analyzer (ABX Diagnostics, Irvine, Calif.). Platelet yields were calculated by multiplying the platelet count times the volume of the platelet unit. After storage, in vitro measurements of glucose and lactate concentration, and pH at 4° C. were measured with a commercially-available blood gas instrument (Radiometer Medical, ABL Flex 805, Copenhagen, Denmark). Annexin V binding, P-selectin expression, microparticle quantification and mean platelet volume (MPV) were performed by flow cytometry (FACSCalibur, Beckman Coulter, Indianapolis, Ind., USA) as previously described. Kunicki et al., Transfusion 1975; 15: 414-421. The following antibodies were utilized: P-Selectin CD62P-FITC (BD Biosciences, San Jose, Calif., USA); GPIbα-PE (BD Biosciences, San Jose, Calif., USA); and Annexin-V-FITC (BD Biosciences, San Jose, Calif., USA).

Statistical analysis. Results are reported as mean±one standard deviation, and statistical significance was assessed by unpaired, 2-tailed Student t test, unless otherwise indicated. A P value equal or less than 0.05 was considered significant.

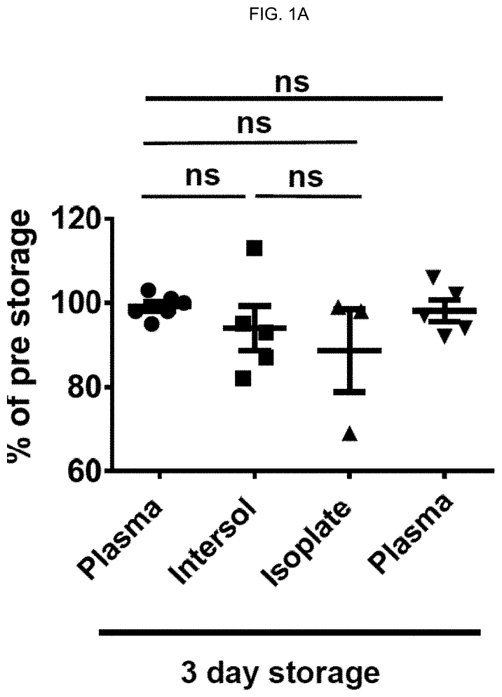

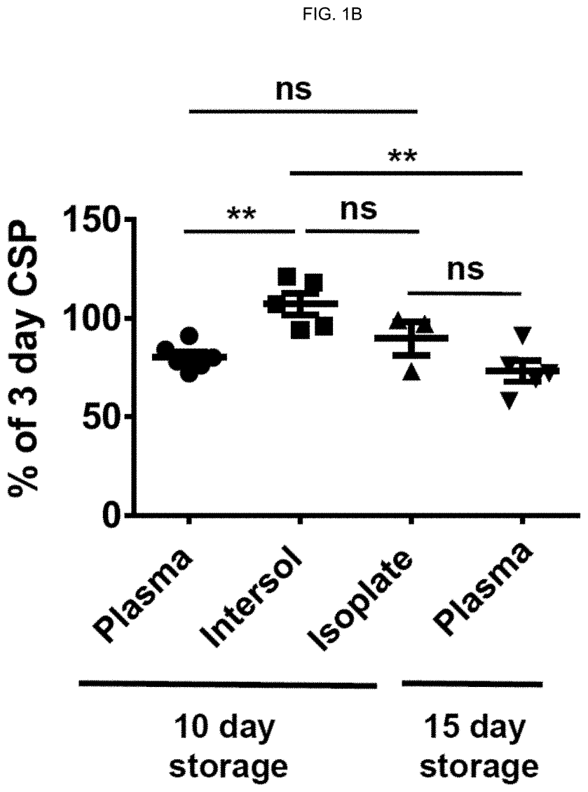

Results. Total platelet yield after storage. Post-storage platelet counts of the “control” units averaged 3.57±0.38×10¹¹ post-storage (99±3% of pre-storage values). No significant differences in platelet yields between PAS and plasma stored platelets were seen after 3 days of storage (FIG. 1A). For the “test” 10-day plasma, Intersol, and Isoplate, and 15 day plasma units, post-storage platelet counts averaged 2.86±0.39×10¹¹, 2.82±0.35×10¹¹, 2.52±1.28×10¹¹, and 2.40±0.36×10¹¹, respectively (80±7%, 107±12%, 90±39%, and 72±14% of pre-storage values). A significant reduction after 10 day and 15 day storage in plasma when compared to 10 day PAS Intersol-stored platelets (p=0.001 and p=0.002, respectively) was found. Platelet yields of 10 day plasma-stored, Intersol-stored, and 15 day plasma-stored did not differ significantly when compared to 10 day Isoplate-stored platelets either absolute or as percentage of 3 day CSP (FIG. 1B). Overall, these studies demonstrate that storing platelets in plasma might have the disadvantage of losing platelets either to the wall of the bag or by micro-aggregation of platelets mediated by fibrinogen as described previously by Getz et al., Transfusion 2016; 56: 1320-1328 (FIG. 1B).

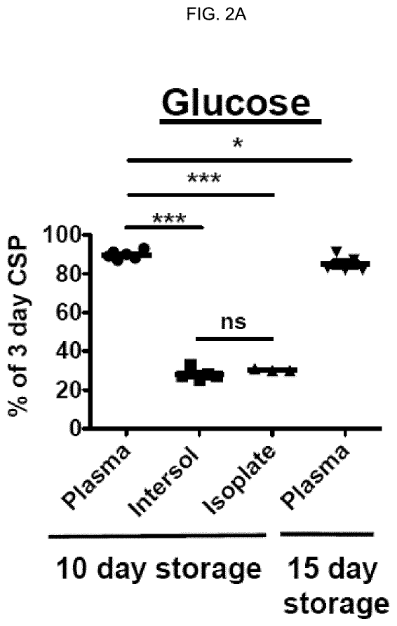

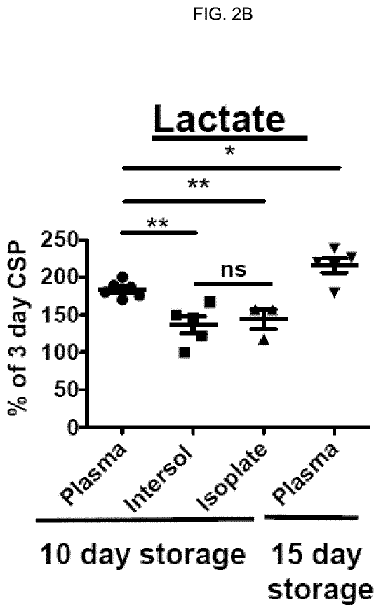

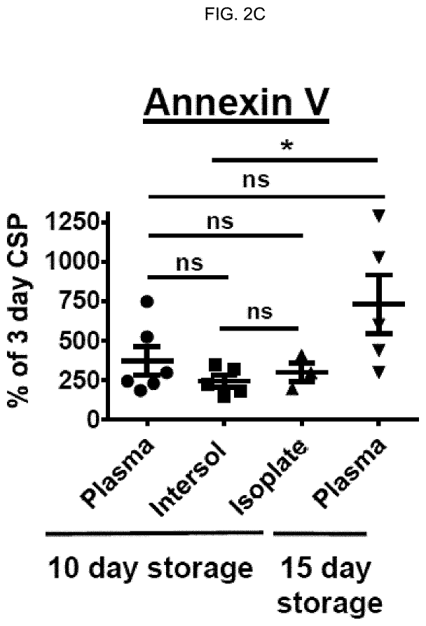

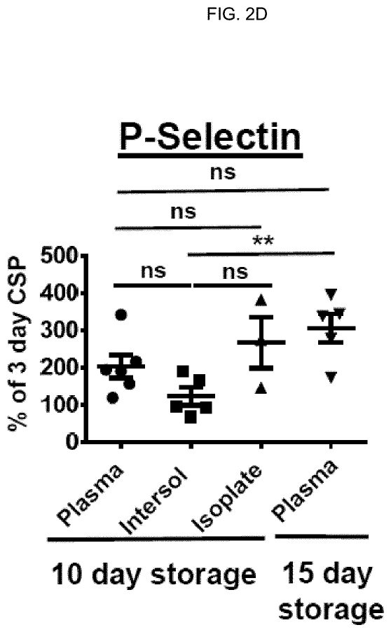

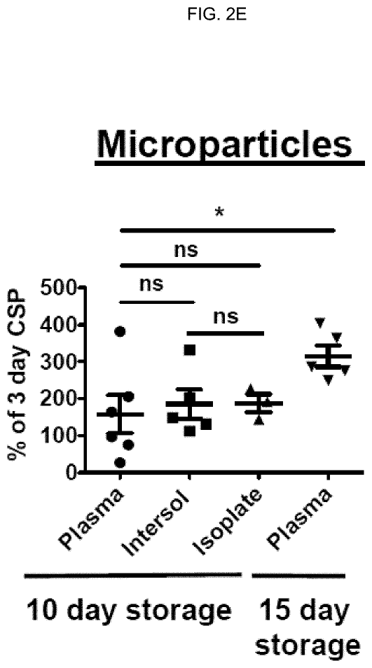

In vitro platelet measurements. As expected, glucose levels were significantly lower in either Intersol or Isoplate PAS compared to plasma stored units (FIG. 2A, 10 day plasma, Intersol, Isoplate, 15 day plasma: 321±61 mg/dl, 99±9 mg/dl, 82±41 mg/dl, 321±81 mg/dl, [90±2%, 28±3%, 24±12%, 86±4% of day 3 storage values, respectively], p<0.0001 for Intersol and Isoplate versus 10 day plasma-stored units) and, inversely, lactate levels were significantly elevated in plasma stored units compared with either Intersol or Isoplate stored units (FIG. 2B, 10 day plasma, Intersol, Intersol, 15 day plasma: 8.1±1.3 mmol/l, 4.4±0.7 mmol/l, 4.0±1.9 mmol/l, 9.2±1.5 mmol/l, [184±11%, 137±26%, 119±54%, 222±10% of day 3 storage values, respectively] p=0.003 for 10 day plasma versus Intersol and p=0.007 for 10 day plasma versus Isoplate). Lactate was also significantly higher in plasma stored for 15 days compared to 10 days of plasma storage indicating ongoing metabolic activity in plasma (FIG. 2B, p=0.012). The removal of plasma (which contains glucose) during preparation of the PAS/plasma units was likely the reason for the lower amount of glucose.

Lower glucose was also likely the reason for the lower lactate, as removal of plasma with glucose deprived them of the essential energy source. It is also possible that platelets in PAS are less metabolically active compared with platelets stored in plasma, however, this will require further investigation. Markers indicating membrane orientation changes with phosphatidyl serine exposure (% Annexin V-binding, FIG. 2C) only showed a significant difference between Intersol and 15 day plasma (p=0.034) (FIG. 2C, 10 day plasma, Intersol, Isoplate, 15 day plasma: 16.5±11.7%, 16.1±5.1%, 15.8±3%, 26.5±12.4% [372±219%, 246±87%, 301±106%, 730±413% of day 3 storage values], respectively). There was a trend for lower degranulation as measured by platelet alpha granule secretion (P-selectin expression) with Intersol when compared with 10 day plasma which did not reach statistical significance (p=0.08), however when Intersol was compared to 15 day plasma a significant difference was detected (p=0.004). (FIG. 2D, 10 day plasma, Intersol, Isoplate, 15 day plasma: 31±28%, 21.8±38.6%, 48.6±21.5%, 40.2±15.3%, [203±76%, 123±53%, 267±118%, 278±102% of day 3 storage values], respectively). Microparticles were significantly elevated in 15 day plasma compared to 10 day plasma-stored platelets (p=0.03) (FIG. 2E, 10 day plasma, Intersol, Isoplate, 15 day plasma: 1.7×10⁵±1.6×10⁵/μL, 6.1×104±3.5×10⁴/μL, 7.4×10⁴±2×10⁴/μL, 4.1×10⁵±2.1×10⁵/μL, [158±127%, 186±88%, 188±41%, 561±605% of day 3 storage values], respectively), but there was no significant difference between plasma and PAS solutions at 10 days of storage. For all platelet activation parameters there was either a trend or a significantly higher value in 15-day plasma-stored units compared with 10 day plasma-stored units indicating continuous in vitro activation of platelets in plasma.

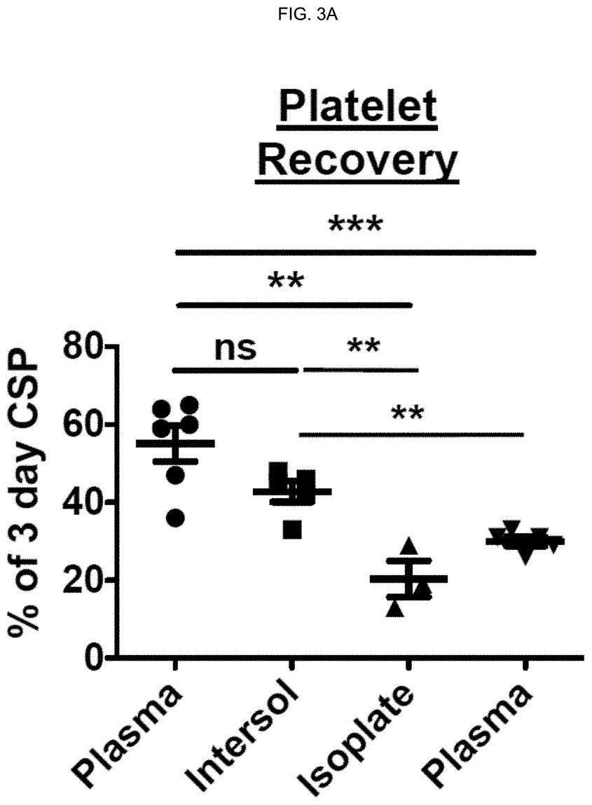

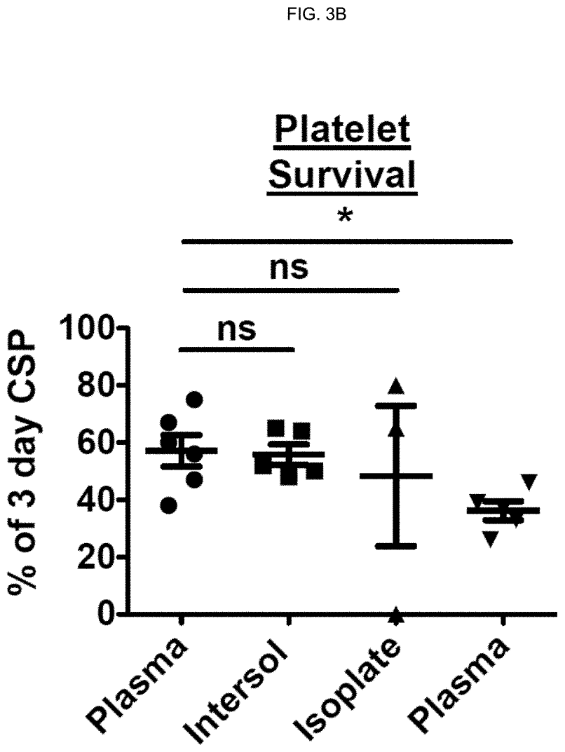

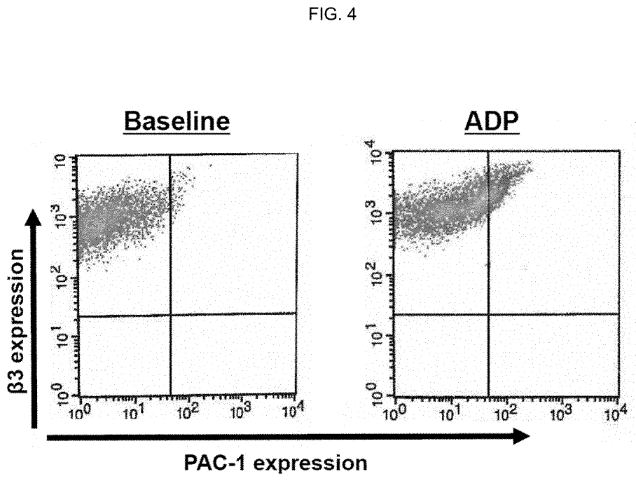

In vivo platelet viability. For the 3 day “control” units (n=19), post-storage platelet recoveries averaged 43±11% and survivals 2±0.4 days. For the “test” 10-day plasma, Intersol, Isoplate, and 15 day plasma units, post-storage recoveries averaged 24±8%, 18±4%, 8±2%, and 11±3% respectively (FIG. 3A, 55±11%, 43±6%, 21±8%, and 29±3%, respectively of the same subject's 3 day control data). The recovery of 10-day Isoplate and 15 day-stored plasma platelets was found to be significantly lower compared with both 10-day plasma and Intersol-stored platelets (FIG. 3A, p=0.002 and p=0.004 for 10 day Isoplate versus 10 day plasma and Intersol respectively, as well as p=0.001 and p=0.002 for 15 day plasma versus 10 day plasma and Intersol respectively). Interestingly, there was no significant difference between the recoveries of 10-day Intersol and plasma stored platelets, although there was a trend for a lower recovery with Intersol (FIG. 3A, p=0.057). Notably, Isoplate-stored platelets showed significantly lower recoveries compared with Intersol-stored platelets (p=0.004). Post-transfusion survivals for the 10-day CSP stored in plasma, Intersol, Isoplate, and 15 days in plasma averaged 1.2±0.3 days, 1.1±0.3 days, 0.9±0.8 days, and 0.7±0.2 days respectively (FIG. 3B, 59±12%, 56±8%, 48±42%, and 37±7%, respectively of the same subject's 3 day data). Platelet in vivo survival studies showed a significantly lower survival with platelets stored in plasma for 15 days compared to any of the 10 day stored platelets (FIG. 3B, p=0.01), but there were no significant differences among the 10 day stored platelet groups.

Discussion Example 1 provides four major findings to highlight: First, the platelet yield is significantly lower in plasma compared with platelets stored in Intersol. Second, cold-stored platelets are metabolically active and consume glucose, produce lactate, and show signs of increasing pre-activation. Third, different PAS solutions can yield significantly different in vivo results which cannot be predicted by their in vitro results. In the present study, Isoplate showed a significantly lower recovery, and a significantly lower platelet yield compared with Intersol, even though both have in vitro parameters mostly in the same range. Fourth, platelet recovery appears to be better in plasma compared with PAS solutions, even though the platelet yield in vitro is significantly higher with Intersol compared with plasma.

In vivo survival is likely not a major factor for cold-stored platelets, since the current target patient population for use of these platelets are those with active bleeding, who are in need of platelets to facilitate immediate localized hemostasis. Platelet survivals did not differ among any of the 10-day CSP but was significantly less at 15 days.

Previous in vivo animal imaging studies suggest that hemostasis and local clot formation are processes which require minutes (3-20 min) and are not accomplished within seconds. Stolla et al., Blood 2011; 117: 1005-1013. The one-hour in vivo platelet recovery measurement is likely an important parameter for transfused platelets since, in order to be hemostatically active, some platelets need to circulate for at least 60 min. In addition, the time-frame required to complete major surgery itself is likely to require several hours. One of the major findings is that there are significant differences between PAS solutions for cold-storage. While platelets collected in Intersol showed recoveries which were not significantly different than plasma (p=0.057), Isoplate showed significantly lower recoveries (p=0.002). Previous studies suggest that platelet function is better preserved during cold-storage in PAS and that there is less platelet aggregate formation in PAS compared with plasma. Getz et al., Transfusion 2016; 56: 1320-1328. A lower platelet yield in plasma compared with PAS solutions was observed, but overall platelet recoveries were better in plasma compared with PAS. This either suggests that any potential aggregates which pass the bedside transfusion filter disaggregate in vivo and are of no clinical significance, or that the aggregates are filtered out by the transfusion filter and platelets that pass the bedside transfusion filter have a higher recovery compared with PAS stored platelets.

A recently presented randomized Norwegian pilot study of 20 patients in each arm showed overall less blood loss in cardiac surgery patients after chest closure with cold-stored platelets stored in the PAS T-PAS (Terumo BCT, Denver, Colo.) compared with RT-stored platelets. Manno et al., Blood 1991; 77: 930-936. T-PAS was not investigated because it currently is not available in the US.

Better in vivo function measured by reduced blood loss in surgery patients has yet to be demonstrated with plasma-stored CSP.

It is acknowledged that in the current studies, collection of platelets in Intersol and Isoplate was conducted on the TRIMA® system [TRIMA is licensed for use with Isoplate, while Intersol requires collection with the Fenwal Amicus system (Fresenius Kabi, Bad Homburg, Germany)]. It is not believed that this difference was a major determinant in the study.

Should cold-stored platelets be considered for transfusion support in massively bleeding patients, the additional plasma in the plasma-stored unit may be of importance. Recent studies have suggested that a higher plasma to red cell ratio (1:1:1; plasma, platelets, RBCs respectively) could be beneficial in trauma patents. Holcomb et al., JAMA 2015; 313: 471-482. Platelet units in plasma are simpler to prepare than units which require replacement of plasma with PAS in a very specific ratio, a significant factor given that these platelets are currently targeted as a future primary choice for blood banks in remote locations and far forward military scenarios. Patients who are in need of functional platelets are frequently coagulopathic as well and could benefit from the additional plasma in the platelet unit, and the minor platelet loss due to microaggregates during storage in plasma may not outweigh the loss of plasma when opting for the replacement with PAS. It should also be emphasized that while one group reported visible aggregates in platelet units stored in plasma at 4° C. (Getz et al., Transfusion 2016; 56: 1320-8), visible aggregates were not observed in the plasma stored units described herein. The only evidence for microaggregates in the disclosed study was based on the lower platelet yield in plasma versus PAS. However, data (not shown) suggests that this loss is in part due to loss of platelets that adhere to the bag walls.

In summary, Example 1 describes the first to compare recovery and survival of CSP in PAS and plasma in healthy human subjects. It was found that both 65% Intersol/35% plasma and 100% plasma CSP have potential advantages over the other.

### Example 2

Effects of storage time prolongation on in vivo and in vitro characteristics of 4° C.-stored platelets. Bacterial contamination of platelet products for transfusion is a major safety problem in blood banking. Cold-storage of platelets could alleviate these issues because of very limited bacterial growth at 4° C. Unfortunately, refrigeration of platelets is known to reduce platelet circulatory lifespan to 1 to 2 days compared to an average lifespan of 4 to 5 days at 22° C. storage (Murphy & Gardner. N Engl J Med. 1969; 280(20):1094-1098). Previous reports suggested superior in vitro performance of platelets stored in the cold (Becker et al. Transfusion. 1973; 13(2):61-68; Reddoch et al. Shock. 2014; 41 Suppl 1:54-61). However, earlier studies about bleeding time corrections after transfusion of cold-stored platelets in thrombocytopenic patients yielded contradictory results. In addition, they were performed in the 1960s and 1970s and require confirmation in contemporary platelet transfusion settings including clinical trials (Becker et al. Transfusion. 1973; 13(2):61-68; Filip & Aster. J Lab Clin Med. 1978; 91(4):618-624; Slichter & Harker. Br J Haematol. 1976; 34(3):403-419). Other unresolved issues around 4° C. storage include determination of the maximum storage time, evaluation of appropriate platelet quality markers, and acceptable storage media. Blood banks in the US can currently apply for a FDA variance, which allows for 3 day cold-storage in plasma for actively bleeding patients, which is based on a historic 3-day storage time limit.

For licensing purposes, the FDA requires recovery of 66% and survival of 58% of a healthy human subject's fresh platelets for RT-stored platelet products (Center FaDA, Research FBEa. FDA Blood Products Advisory Committee Mar. 13-14, 2003; Center FaDA, Research FBEa. Workshop on use of radiolabeled platelets for assessment of in vivo viability of platelet products, May 3, 2004; Vostal J G. FDA guidance for industry: for platelet testing and evaluation of platelet substitute products. 18th meeting of the BEST Working Party of the ISBT, SF Nov. 5, 1999). Whether these guidelines need to be revised for alternative platelet/hemostatic products is currently under discussion.

It was previously shown that CSP stored in plasma show a better recovery in vivo compared with platelets stored in PAS licensed in the US (Example 1; Stolla et al. Transfusion. 2018; 58(10):2407-2413). Based on these results, it was concluded that platelets stored in plasma show promise since they (i) yield a higher recovery compared with US-licensed PAS (Stolla et al. Transfusion. 2018; 58(10):2407-2413), (ii) are intended for actively bleeding trauma and surgery patients, and (iii) are easier to manufacture in far forward military scenarios.

The goal of this study was to characterize platelet in vivo and in vitro function at 4° C. storage in plasma at 5, 10, 15, and 20 days and compare these data with a fresh sample, and the current clinical standard. To validate 20 day, cold- and plasma stored platelets as a transfusion product, platelets were stored in plasma 5, 10, 15 and 20 days at 4° C. and included a fresh and 5 day RT comparator. The study provides essential guidance for future clinical trials.

Platelets were subjected to several state-of-the-art tests for platelet in vitro and in vivo quality and function. Tests to assess mitochondrial health and apoptosis were also included to better understand changes in platelet biology during storage at 4° C. and to further characterize the cold lesion.

The study sheds light on in vivo and in vitro features of platelets stored up to 20 days in plasma. Twenty-day storage essentially quadruples the current platelet shelf-life for RT-stored platelets. The in vitro data suggests that 20-day CSP are not inferior and may even be superior in trauma or surgery patients compared with 5 day RT platelets.

This is the first in human study to investigate the storage and in vivo characteristics of extended storage, cold-stored platelets in plasma up to 20 days.

Materials and Methods. Preparation of Control and Test platelets. A standard single apheresis platelet unit (target platelet yield 3.0×10¹¹/unit and concentration of 1500×10³ platelets/μL) was collected from 23 healthy subjects using the Trima Accel Automated Blood Collection System (TerumoBCT, Denver, Colo.). Each unit was calibrated to a final platelet concentration of 700-2100×10⁶ platelets/μL, as per allowable bag parameters. Each calibrated unit was allowed to rest at room temperature for 1 hour without agitation before transfer to cold storage at 4° C. for a predetermined period of up to 20 days, or was stored up to 5 days at RT (22° C.). Cold-stored bags were not agitated. RT-stored bags were agitated as per standard requirement for clinically used units. At the day of testing and transfusion the units were allowed to equilibrate to room temperature before a sample for radiolabeling was taken from the unit. Units were inverted but not otherwise manipulated physically (massaged or shaken) to remove aggregates.

At the end of the storage period, the subject returned to receive an ¹¹¹Indium Oxine (Indium 111, In-111) radiolabeled aliquot of their 4° C. stored platelets. Follow-up samples from the subject were collected 2 hours post-transfusion (recovery) and on Days 1 (2×), 2 (2×), and 3 to calculate recovery and survival of the subject's 4° C. stored platelets. The Day 1 and Day 2 sample draws were 2-10 hours apart.

Seven to 14 days after the infusion of the radiolabeled aliquot, the subject returned to receive a second radiolabeled aliquot of fresh platelets. To facilitate this, on the morning of the second infusion, the subject returned for collection of a 43 mL blood sample. The blood was processed to obtain a sample of the subject's platelets to serve as a “fresh” or pre-storage control comparator. The platelets were radiolabeled with In-111. The subject returned later in the day for infusion of the radiolabeled fresh ‘control’ comparator aliquot. Follow-up blood samples were drawn at 2 hours after the infusion on Day 0 and then on Days 1, 2, 3, 4 or 5, and 6 or 7 day post infusion to calculate recovery and survival of the subject's fresh vs. stored platelets.

All RT data were taken from a previously performed and reported study, which was performed under conditions which match this current study in terms of collection concentration, platelet count and storage. The only difference was that for the fresh comparator Indium 111 was used and the radiolabeling compound for the stored platelets was Cr-51. These data have been reported previously to show correlation with platelet metabolites in the storage bag (Zimring et al. Transfusion. 2016; 56(8):1974-1983). Re-utilization of these data avoids unnecessary repetition of experiments in healthy human subjects who have virtually no medical benefit and expose themselves to risks for these procedures. The 5-day RT time point is a common control group for many studies; therefore, it is considered ethically irresponsible to repeat this group with every study as long as collection, storage, and test parameters do not change.

Radiolabeling of stored platelets. Twenty one out of 23 subjects were available for in vivo assessment. Two subjects did not return for the autologous transfusion. Platelets were radiolabeled as previously described, following the detailed Biomedical Excellence for Safer Transfusion (BEST) collaborative protocol ((BEST)Collaborative. TBEfST. Platelet radiolabeling procedure. Transfusion 2006; 46:59S-66S). In brief, Indium (In-111, Anazao, Tampa, Fla., USA) was used to label both control and test platelets because per preliminary experiments, the other available isotope, chromium, demonstrated very poor uptake of Cr-51 by refrigerated platelets. Because of the short circulating times of cold-stored platelets, re-use of the same isotope to measure both control and test cold stored platelets was considered feasible. Additionally, a pre-infusion radioactivity sample was collected prior to the fresh (control) transfusion to account for any possible residual In-111 activity after each subject's control transfusion, and calculations were adjusted accordingly. For the RT data, the fresh comparator was labeled with Indium-111 and the stored group was labelled with Cr-51. Studies in the past have shown that Cr-51 and Indium-111 correlate well for recovery and survival assessment (Keegan et al. Transfusion. 1992; 32(2):113-120).

In vitro platelet measurements. Platelet counts of collected products were performed on the day of collection after 2 hours of resting time to allow platelet disaggregation that might have occurred during collection and at the end of storage using an ABX Hematology Analyzer (ABX Diagnostics, Irvine, Calif.). Platelet yields were calculated by multiplying the platelet count times the volume of the platelet unit. At day 0 and after storage, in vitro measurements of glucose and lactate concentration at 4° C. were measured with a commercially-available blood gas instrument (Radiometer Medical, ABL Flex 805, Copenhagen, Denmark). P-selectin expression was performed by flow cytometry (FACSCalibur, Beckman Coulter, Indianapolis, Ind., USA) as previously described (Kunicki et al., Transfusion 1975; 15:414-421). The following antibodies were utilized: P-Selectin CD62P-FITC (BD Biosciences, San Jose, Calif., USA), GPIbα-PE (BD Biosciences, San Jose, Calif., USA), PAC-1 (BD Pharmingen, San Jose, Calif., USA) binding was tested by flow cytometry as previously described (Voss et al. Mol Pharmacol. 2007; 71(5):1399-1406) at baseline and after stimulation with 20 μg/ml collagen (Chrono-Log, Havertown, Pa., USA) for both fresh and stored unit. Caspase 3,7 activation was measured as previously described (Kim et al. Haematologica. 2019; haematol-2018). In brief, a commercially available CellEvent™ (Life Technologies Corporation, Carlsbad, Calif.) Caspase-3/7 Green Detection Reagent was incubated with platelets with or without ABT 737 and subsequently read on a flow cytometer for FL-1. For mitochondrial membrane potential measurements, the commercially available JC-1 dye (Invitrogen, Carlsbad, Calif.) was utilized as previously described (Verhoeven et al. The mitochondrial membrane potential in human platelets: a sensitive parameter for platelet quality. Transfusion. 2005; 45(1):82-89). In brief, JC-1 is a positively charged probe which accumulates in the negatively charged internal environment of healthy mitochondria and forms red fluorescent J-aggregates if concentrated. Upon depolarization the J-aggregates dissipate and green fluorescence can be detected. The FI1/FI1 ratio therefore helps to determine if the dye is concentrated in intact mitochondria or leaking into the cytoplasm.

Healthy human subjects research. The research was approved by the Western Institutional Review Board (WIRB) and all human participants gave written informed consent. The clinical trial registration number for this study was NCT02754414 and the entry name: Cold Apheresis Platelets in PAS (CAPP). The study was conducted in accordance with the Declaration of Helsinki.

Statistical analysis. Based on a previous study which evaluated extended RT-storage with platelet additive solutions (Slichter et al. Blood. 2014; 123(2):271-280), an effect size of 2 for platelet recoveries between different time points was calculated. Based on this a sample size of n=5 was calculated to be required to provide 80% power to detect a statistically significant difference between the different plasma time points (t-test, unpaired, two-tailed, a err. prob. 0.05). Therefore, 5 subjects were enrolled per group and then assessed for statistical significance. No additional data points were obtained after the study was concluded and tests for significance were run. Results are reported as mean±standard error of the mean and statistical significance was assessed by unpaired, 2-tailed Student t test, unless otherwise indicated. A p value equal or less than 0.05 was considered significant.

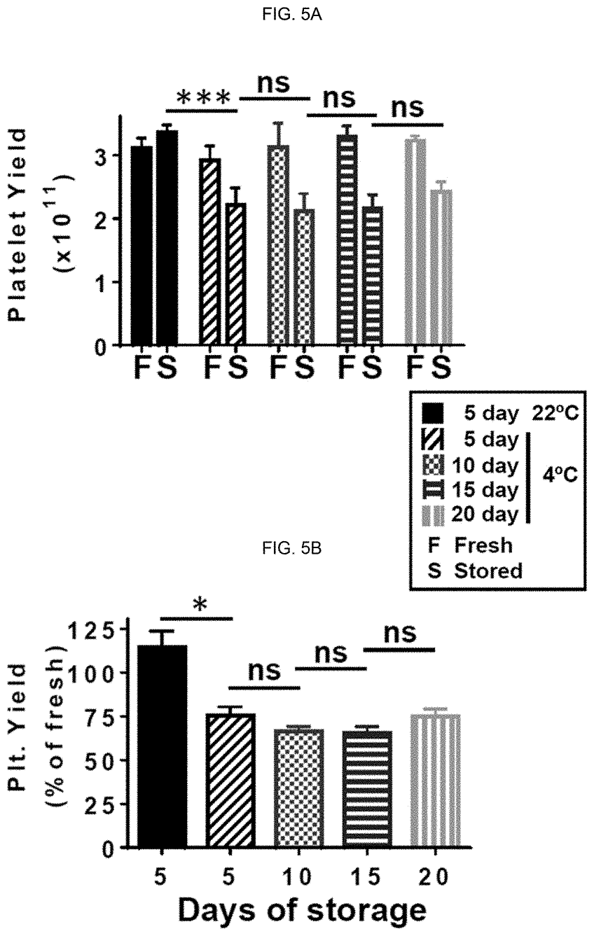

Results. Total platelet yield after storage. Post storage platelet yields averaged 3.36±0.12×10¹¹ for 5-day RT-stored platelets. A significantly lower platelet yield was observed for platelets stored for 5 days in the cold (2.21±0.27×10¹¹, p=0.0003). After 5 days no significant differences were observed between 5 to 10 days, 10 to 15 days, and 15 to 20 days (FIG. 5A) either as absolute or percentage of fresh. Five-day 4° C.-stored platelets showed a significantly lower yield compared to 5-day 22° C. (114±9% of pre-storage values vs. 75±5%, p=0.046) (FIG. 5B).

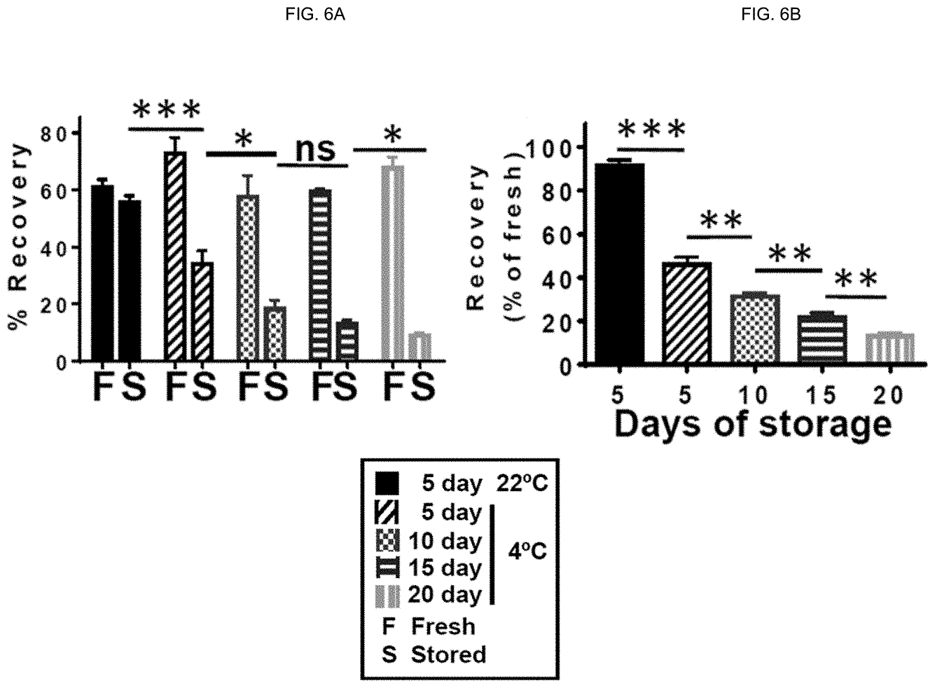

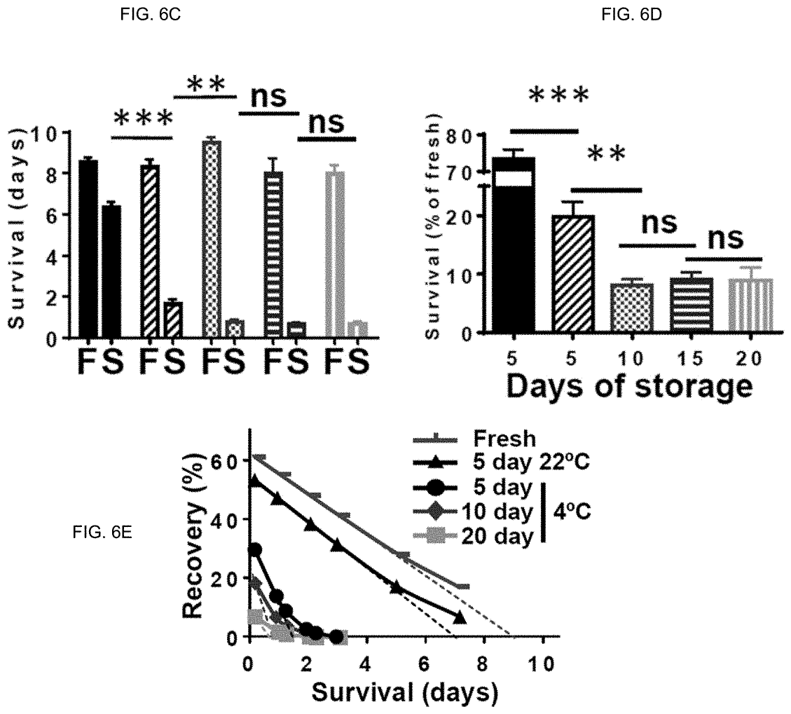

In vivo platelet viability. There was a significant step-wise decrease in recovery of stored platelets from 5-day to 10-day 4° C. (34±5% vs. 18±3%, p=0.016), and 15 to 20 day 4° C. (13±1% vs. 9%±1%, p=0.041) (FIGS. 6A, 6E). Cold storage of platelets led to a reduction of recoveries when compared to RT storage which was significant after 5 days of storage (53±2% vs. 34±5%, p=0.0007). When the percentage of fresh results was calculated, all steps were significantly lower compared to the respective previous step, and 5 day RT was significantly lower compared to 5 day CSP (92±3% vs. 46±3%, p<0.0001, 46±3% vs. 31±2%, p=0.003, 31±2% vs. 22±2%, p=0.009, 22±2% vs. 13±1%, p=0.008) (FIG. 6B).

Platelet survival studies showed a significantly lower survival of refrigerated platelets compared to RT-stored platelets. Interestingly, the maximum drop occurred early (between 0 and 10 days) and after 10 days a low plateau was reached. This was reflected in both the absolute analysis and the percentage of pre-storage analysis which both showed significant drops between 5 day RT and 5 day cold (6.6±0.3 days vs. 1.6±0.2 days, p<0.0001, 74±3% vs. 20±3%, p<0.0001) and 5 day cold and 10 day cold (1.6±0.2 days vs. 0.8±0.1 days, p=0.004, 20±3% vs. 8±1%, p=0.001), but no significant further decrease thereafter (FIGS. 6C-6E).

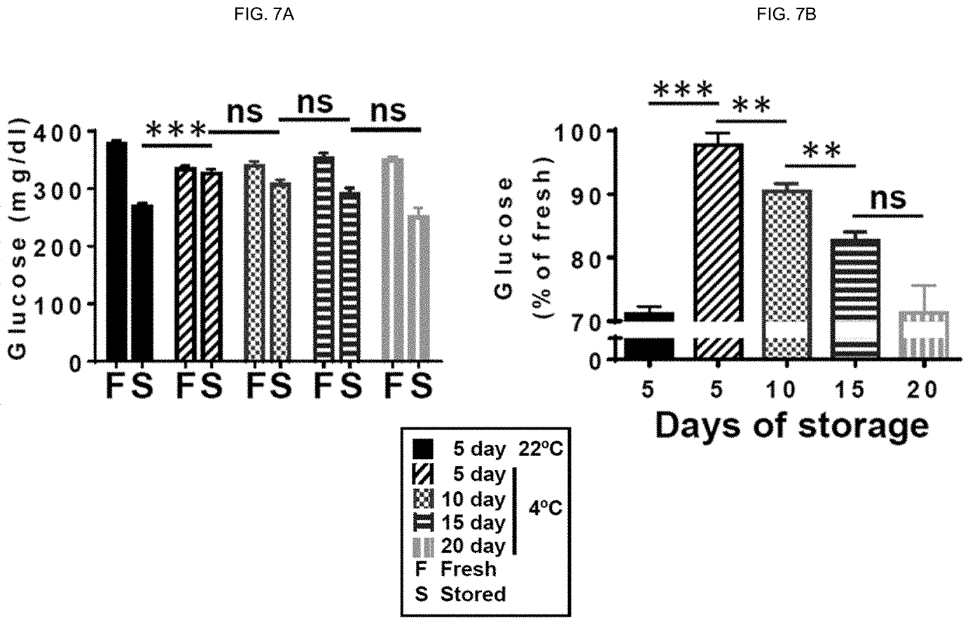

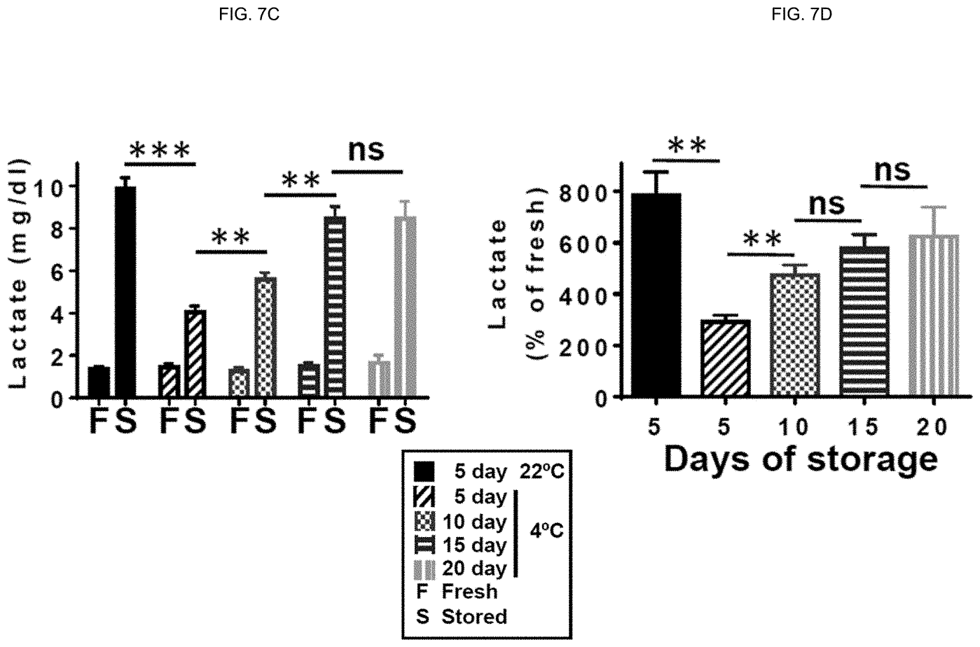

Platelet metabolism markers. Glucose levels dropped quickly and significantly in RT-stored samples when compared to 5 day cold-stored samples (326±7 mg/dl vs. 378±6 mg/dl, p=0.003) (FIG. 7A). In contrast, there was a step-wise decrease in cold-stored platelets from 5 to 20 days of storage, which was not significant (when calculated comparing step by step) in the absolute analysis, but significant between 5 and 10 day, and 10 and 15 days in the percentage of pre-storage analysis (98±2% vs. 90±1%, p=0.007, and 90±1% vs. 71±4%, p=0.05 respectively) (FIG. 7B). Correspondingly, lactate increased more rapidly at RT and was significantly higher than 5 day cold-stored platelets (9.9±0.5 mg/dl vs. 4±0.3 mg/dl, p<0.0001). Lactate in cold-stored platelets mirrored the glucose measurements with a step wise, significant increase from 5 to 10 days (4±0.3 mg/dl vs. 5.6±0.3 mg/dl, p=0.0064), and 10 to 15 days (5.6±0.3 mg/dl±8.6±0.4 mg/dl, p=0.0009), but after 15 days a high plateau was reached.

Platelet viability markers in vitro. Platelet αIIbβ3 integrin activation requires inside out signaling and is critical for platelet participation in hemostasis at the site of vascular injury. Therefore, platelet activation was tested in response to collagen, an extracellular matrix protein which can cause platelet adhesion under flow and platelet aggregation via the ITAM (immunoreceptor tyrosine-based activation motif) receptor GPVI and the αIIβ1 integrin (Boulaftali et al. J Clin Invest. 2013; 123(2):908-916). Platelet αIIbβ3 integrin activation was measured with the PAC-1 antibody which specifically binds the activated conformation of the integrin (Shattil et al. J Biol Chem. 1985; 260(20):11107-11114).

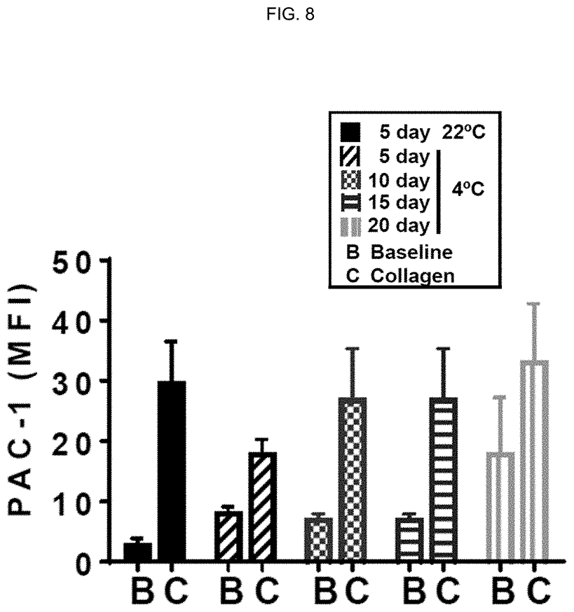

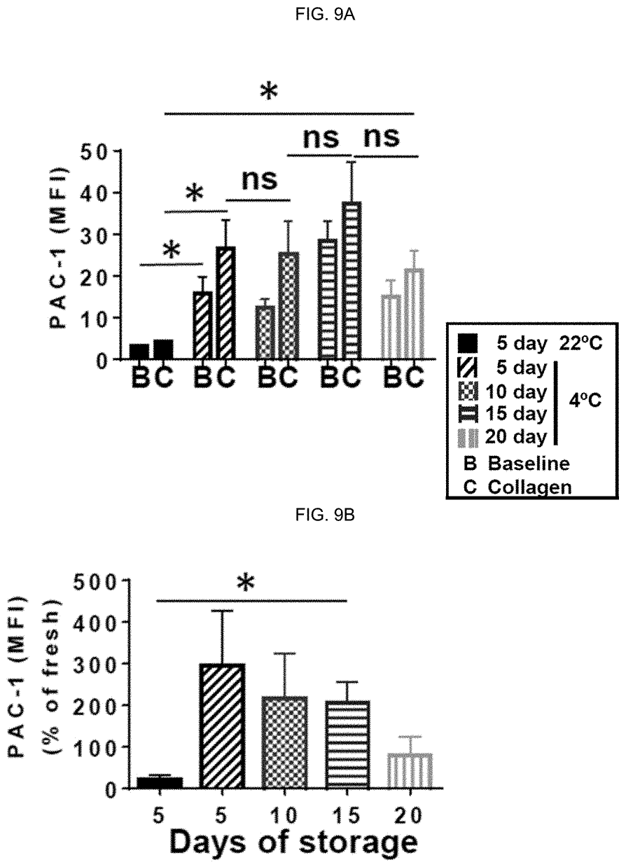

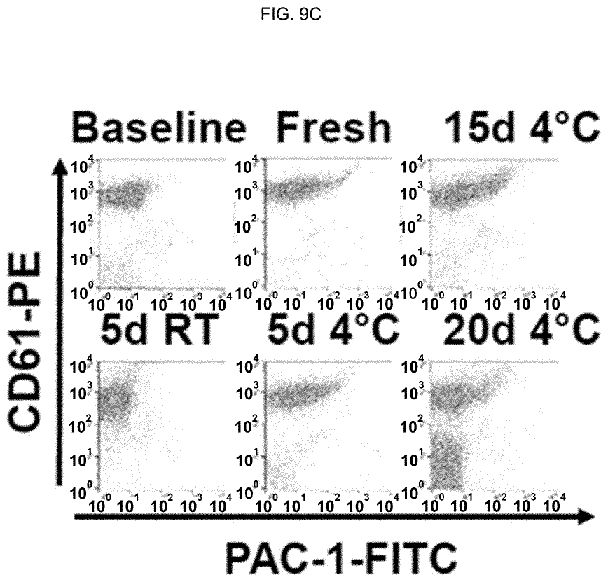

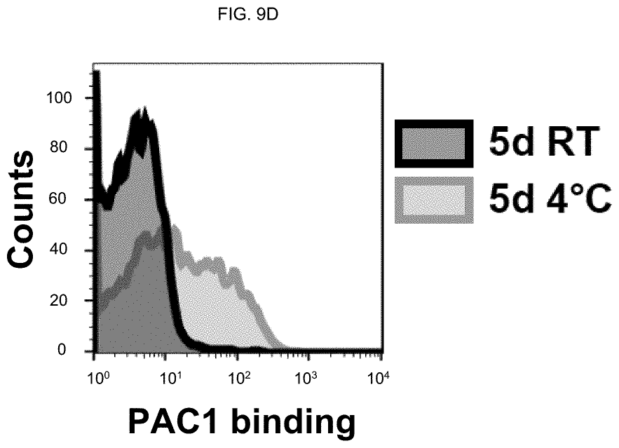

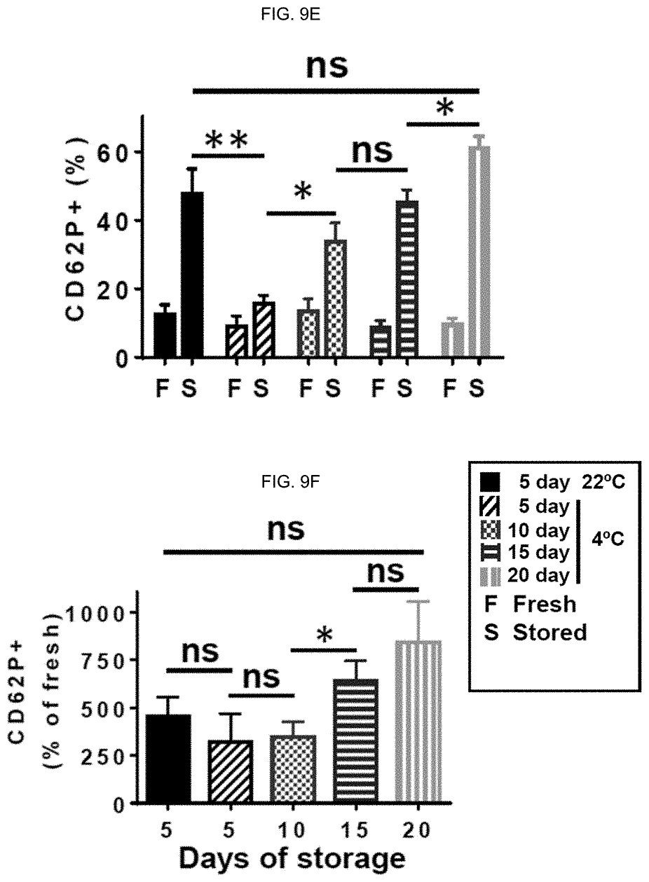

All samples responded appropriately to collagen before storage (fresh) (FIG. 8). As described previously, pre-activation of αIIbβ3 integrin at 5 days of cold-storage was observed (FIG. 9A), when compared to pre-storage (FIG. 8). No further increase of pre-activation was noted between 5 and 20 days (FIG. 9A). The pre-activation level was higher in 5 day cold-stored platelets compared to five-day RT-stored platelets (3.2±0.22 vs. 15.7±4.1, p=0.038). Five day cold stored platelets showed a significantly greater response to collagen compared to 5 day RT-stored platelets (26.7±6.6 vs. 4.4±0.3, p=0.018) (FIG. 9A). The integrin activation in response to collagen was significantly lower in 5-day RT-stored platelets compared to cold-stored platelets even after 20 days of storage (21.3±4.8 vs. 4.4±0.3, p=0.012) (FIG. 9A). When the activation response of the corresponding fresh platelets was taken into account a significantly greater response was noted in 15 day cold-stored platelets compared to 5-day RT-stored platelets (FIG. 9B). The corresponding scatter plots and histograms showed a clear increase in PAC-1 binding in cold-stored platelets which was comparable to fresh platelets and a lack of PAC-1 binding in RT-stored platelets comparable to unstimulated samples (FIGS. 9C, 9D).

Platelet α-degranulation is a hallmark of platelet storage lesion. P-selectin expression over time in the storage bag (without agonist stimulation) was tested. RT storage leads to a significantly greater P-selectin expression at 5 days (47.6±7.4 vs. 15.7±2.5, p=0.005) compared to 5 days cold-storage. However, at refrigerated temperatures, there was a continuous increase in degranulation and by day 20 the level was similar, and not significantly different compared to 5-day RT-stored platelets, either in absolute numbers or as percentage of fresh (47.6±7.4 vs. 61±3.5, p=0.134, and 457±98% vs. 841±215%, p=0.135) (FIGS. 9E, 9F).

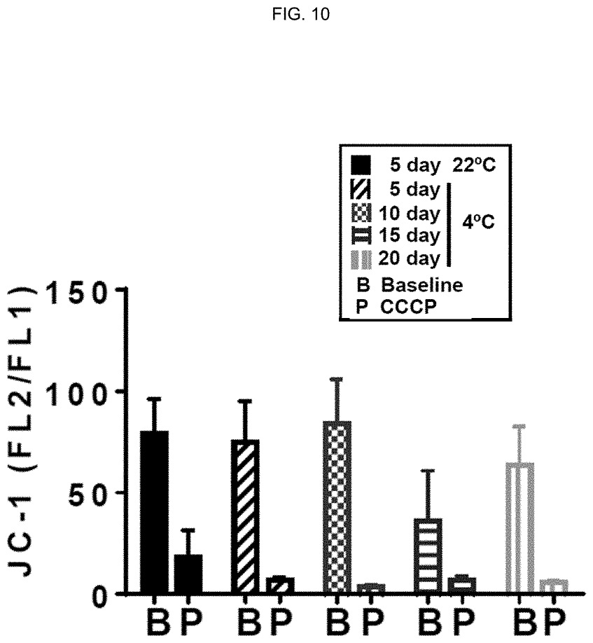

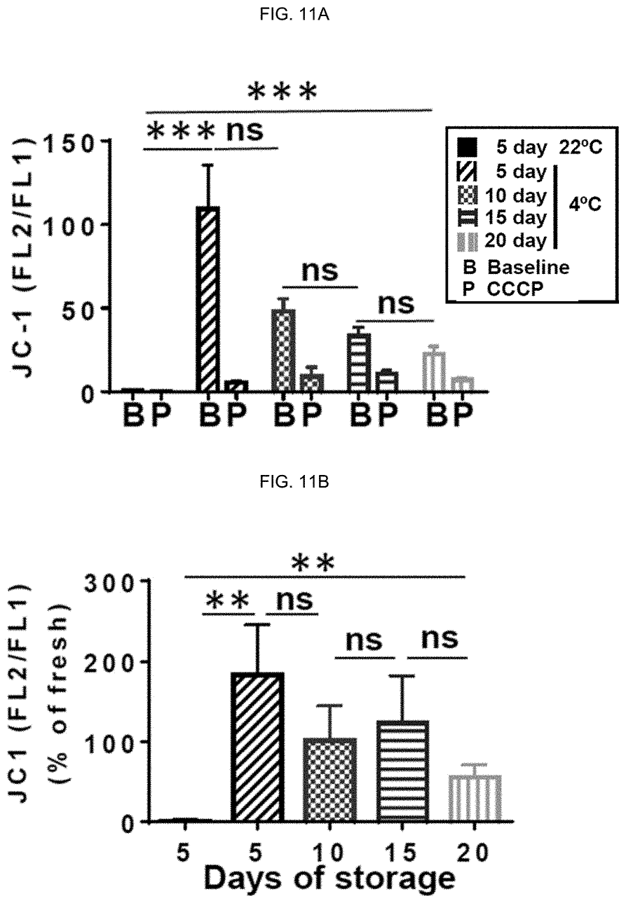

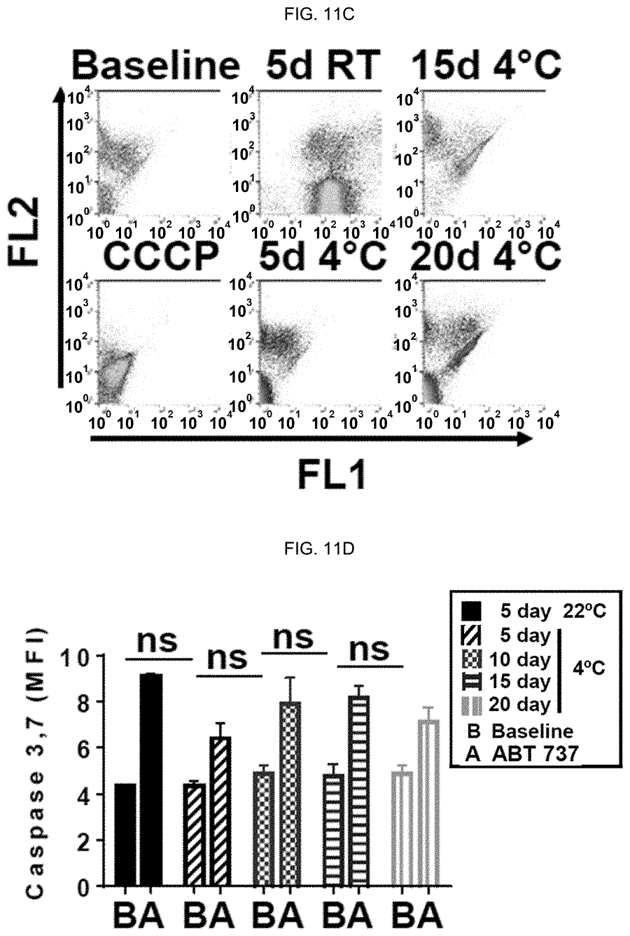

To see how early apoptosis events compared between cold-storage and RT-storage, mitochondrial membrane integrity and caspase 3,7 activation were tested. Mitochondrial membrane integrity was assessed by flow cytometry utilizing the JC-1 dye. When the dye is highly concentrated in mitochondria, it forms J-aggregates with an emission maximum at 590 nm (FL2); when more diluted in cytoplasm, the dye emits at 530 nm (FL1). The proton gradient uncoupler carbonyl cyanide m-chlorophenyl hydrazine (CCCP) was utilized as positive control to show successful disruption of the electron transport chain. All fresh samples showed a significant drop of the FL2/FL1 ratio after CCCP was added to the sample (FIG. 10). When testing stored samples a markedly reduced FL2/FL1 ratio in 5 day RT-stored platelets was observed, compared to cold-stored platelets at 5 days (109.5±25.9 vs. 1.1±0.5, p=0.0005) (FIG. 11A). Surprisingly, this difference remained significant for the maximum storage time tested (20 days), indicating a significant advantage of cold-storage for mitochondrial preservation (22.7±4.5 vs. 1.1±0.5, p=0.0003) (FIGS. 11A, 11C). Of note, cold platelets responded appropriately to CCCP and a reduction in FL2/FL1 ratio was observed up to 20 days of storage, while the ratio was already too low at baseline to appreciate a reduction in RT-stored platelets (FIG. 11A). The percentage of fresh analysis corroborated the absolute analysis and significant differences were found between 5 day RT and 5 day 4° C. and 5 day RT and 20 days of cold-storage (1.7±0.8 vs. 183.5±62.4, p=0.009, and 1.7±0.8 vs. 55.3±15.9, p=0.004, respectively) (FIG. 11B).

During cell stress mitochondrial membrane disruption is followed by caspase 3,7 activation (McArthur et al. Blood. 2018; 131(6):605-610). Surprisingly, this sequence was not found in 5 day RT, or cold-stored platelets at any time point. While there was a trend for higher caspase 3,7 activation in 10 and 15 days of cold-storage in the percentage of fresh analysis, no significance was observed for any time point between the groups (FIGS. 11D, 11E). Importantly, all samples showed an appropriate increase in caspase 3,7 activation in response to the Bcl-2 and Bcl-xL inhibitor ABT 737, indicating the general capacity to undergo apoptosis (positive control) (FIG. 11F).

For this study the occurrence of micro and macroaggregates in cold units was systematically addressed. Overall, over twenty units were included in this study and only one unit had to be discarded because of a large proteinaceous aggregate that had formed over storage time.

Discussion. The present study was based on previous observations suggesting that cold-stored platelets stored in plasma have a higher recovery and survival compared to platelets stored in US-licensed platelet additive solutions (Example 1; Stolla et al. Transfusion. 2018; 58(10):2407-2413).

The study has four major findings: 1) Platelet recovery continues to decline up to 20 days and the trajectory suggests even further decline is possible after 20 days, 2) platelet survival is low after 5 days of 4° C. storage and further decreases to 10 days but then reaches a very low plateau, 3) platelet yield is significantly lower at 5 day 4° C. storage, compared to 22° C. and remains on the same level over 20 days, and 4) markers of platelet biology in vitro indicate that platelets stored for 20 days at 4° C. are equivalent or better compared to 5 day RT platelets.

Cold-stored platelets are evaluated for trauma and surgical patients with active bleeding and therefore platelet survival is not likely to be of high importance. However, since CSP represent a hemostatic transfusion product it is vitally important to characterize its fate in vivo. The study investigated how long CSP stayed in circulation and thus how long a hemostatic effect could be expected. Platelet recovery is a valuable marker for this question because platelets need to circulate until hemostasis is achieved, and severe trauma cases and large surgeries with massive blood loss can take several hours.

The αIIbβ3 integrin activation data suggests that platelet activation after 20 days at 4° C. is significantly better compared to 5 day RT-stored platelets and P-selectin and metabolism parameters are comparable between these time points and storage temperatures. Mitochondrial membrane preservation also remained significantly better at 20 days in the cold compared to 5 days at RT. These findings alleviate potential concerns about platelet quality and the cold-storage storage lesion.

In fact, the maximum αIIbβ3 pre-activation during cold-storage was achieved at the earliest time point (5 days). If there is a cold-priming effect of platelets that renders them more effective for in vivo hemostasis it occurs early and there appears to be little change over time. The response to collagen decreased over cold-storage time suggesting that there are limits for platelet storage at 4° C. The studies using mitochondrial marker and caspase enzyme induction show a peculiar pattern: while mitochondrial membrane potential is clearly affected over time in cold-stored platelets and severely reduced in RT-stored platelets, this did not result in significant caspase activation. This suggests that early stages but not later stages of apoptosis are reached during RT or 4° C. storage. An alternative explanation is that mitochondrial damage occurs independently of a canonical apoptosis pathway activation in platelets during storage. Mitochondrial membrane integrity correlated well with αIIbβ3 activation response to collagen suggesting a causal relationship between mitochondrial health and integrin activation.

The current findings contradict a previously published study by Li et al. who show caspase 3 activation in RT-stored platelets after 7 days (Li et al. Transfusion. 2000; 40(11):1320-1329). While Li et al. see activation already at 3 and 5 days, a different group reports that caspase activation during platelets storage is a relatively late event (Plenchette et al. Leukemia. 2001; 15(10):1572-1581). Therefore, it is possible that the platelets were not stored long enough at both RT and 4° C. to detect caspase activation. The successful caspase activation by ABT 737, however, indicates that the lack of response was not an intrinsic problem with the assay itself.

A continuous decrease in glucose and increase in lactate over storage time was observed, indicating that metabolism continues even though it is markedly slowed when compared to RT storage. Interestingly, 20-day 4° C.-storage showed comparable glucose levels to 5 day 22° C. storage suggesting a metabolism slowed to one quarter of the metabolism at RT. Also in this regard 20 day cold-storage appears to be comparable to 5 day RT.

As described previously in the literature, the yield in cold-stored platelets in plasma is lower likely because of aggregate formation between plasma fibrinogen and pre-activated αIIbβ3 integrins on platelets. The present data suggest that the maximum aggregation happens early during storage (between 0 and 5 days).

The present study suggests platelet integrin activation correlates with hemostatic efficacy in vivo, and further studies can be done to further explore this concept.

To assess hemostatic efficacy in healthy human subjects is notoriously difficult. The bleeding time in response to antiplatelet therapy has often been suggested by some (Filip & Aster. J Lab Clin Med. 1978; 91(4):618-624; Harker & Slichter. N Engl J Med. 1972; 287(4):155-159; Payne et al. J Vasc Surg. 2002; 35(6):1204-1209), but is criticized by others due to a lack of correlation with surgical bleeding (Peterson et al. Arch Surg. 1998; 133(2):134-139). Fecal occult blood is often only present in thrombocytopenic patients and antiplatelet reagents do not reliably lead to detectable fecal blood loss (Ikeda et al. Intern Med. 2014; 53(5):375-381). Furthermore, the short-term effect of a hemostatic product like CSP is likely hard to measure in a fecal occult blood test.

In summary, the present study is the first to compare cold-stored platelets up to 20 days to fresh platelets and to 5-day RT-stored platelets in plasma in healthy human subjects. It was found that platelets stored 20 days in the cold (after a rest period of 1 hour without agitation at room temperature) shared many similarities with platelets stored 5 days at RT, but also have specific advantages and disadvantages.

### Example 3

Evaluation of efficacy and safety of cold-stored platelets in healthy human volunteers. Reversal of dual antiplatelet therapy with acetylsalicylic acid (aspirin) and clopidogrel is currently attempted by transfusion of RT-stored platelets, even though conclusive evidence supporting this practice is missing. Cold-stored platelets were the standard of care in the 1960s and 70s. Cold-stored platelets generally perform better than RT-stored platelets in in vitro, functional assays but studies in humans have thus far yielded contradictory results.

In this randomized, cross-over study, eight healthy adult volunteers who provided a double dose of platelets received a double dose of 5 day cold-stored (after a rest period of 1 hour without agitation at room temperature), or 5-day RT-stored, autologous, and leukoreduced platelets. The first transfusion was followed by a 10-day wash-out period, followed by a second sequence of collection, loading dose and second transfusion of the respective alternate product. The subjects received a loading dose of aspirin and clopidogrel 12-24 h before transfusion. The primary endpoint was post transfusion platelet function in response to agonists inhibited by dual antiplatelet therapy, assessed by aggregometry. Secondary endpoints included platelet function testing by bleeding time, aggregometry to other agonists, platelet function assessed by flow cytometry and commercially available platelet function tests, such as VerifyNOW® (Accumetrics, Inc., San Diego, Calif.), VASP phosphorylation assay (STAGO, Asnieres sur Seine Cedex, France), and CellEvent™ (Life Technologies Corporation, Carlsbad, Calif.).

Platelet transfusions are routinely given to support thrombocytopenic patients, actively bleeding patients, and prophylactically prior to surgery or invasive procedures. For this purpose, platelets are currently stored at 22° C., with gentle agitation for up to 5-7 days.

Stored platelets for transfusion are a limited resource and under high demand for both leukemia and hematopoietic stem cell transplant patients and actively bleeding surgery patients with fundamentally different transfusion demands. While most hematologic patients require prophylactic platelet transfusions with long platelet circulating times to prevent bleeding, actively bleeding patients need therapeutic platelet transfusions for acute hemostasis. Based on a recent survey, a significant number of platelet transfusions go to actively bleeding patients who require acute hemostasis instead of longer platelet survival (Whitaker & Henry. The 2011 National Blood Collection and Utilization Survey Report. 2011).

Cold (2-4° C.)-stored platelets were the standard of care in the 1960-70's, but they were abandoned when radiolabeling studies revealed that the circulation half-life was reduced to 1 day compared to 3-4 days for RT-stored platelets (Murphy & Gardner. N Engl J Med. 1969; 280(20):1094-1098). RT storage has led to a 5-7 day storage time limit because bacterial growth and septic reactions increase rapidly after 5 days. Conversely, storage of platelets at 2-4° C. has the advantage of prolonging storage times while preventing bacterial growth.

Recently updated guidelines by the FDA to prevent septic transfusion reactions from room temperature-stored platelets increases the requirements for bacterial testing or requires pathogen reduction. This will ultimately lead to higher cost and increased labor to provide these products (FDA. Bacterial Risk Control Strategies for Blood Collection Establishments and Transfusion Services to Enhance the Safety and Availability of Platelets for Transfusion. Draft Guidance for Industry. December 2018).

A preliminary analysis of a small pilot trial in Norway showed that CSP stored for seven days in plasma and platelet additive solution reduced postoperative bleeding significantly in patients undergoing major cardiac surgery compared with patients who received RSP (Apelseth et al. Transfusion. 2017; 57(Supplement S3):3-6; P3-A03A). Recently published data suggest that 100% plasma could be more beneficial because of a higher recovery in vivo (Apelseth et al. Transfusion. 2017; 57(Supplement S3):3-6; P3-A03A; Stolla et al. Transfusion. 2018; 58(10):2407-2413).

CSP are mildly activated and it has been speculated that they may be poised to participate immediately in hemostasis, while RSP could require up to 24 h after transfusion to participate in hemostasis in subjects on aspirin (Becker et al., Transfusion 1973; 13: 61-68). CSP retain their in vitro responsiveness to naturally occurring inhibitors of platelet aggregation response, which are released by endothelial cells including nitric oxide, and prostaglandin I₂ (Reddoch et al. Shock. 2016; 45(2):220-227).

The vast majority of in vitro studies highlight the superior function of CSP compared to RSP. Only very few studies investigate CSP after transfusion, but they have thus far yielded contradictory results and were predominantly published in the 1960s and 1970s. Manufacturing practice and storage approaches have changed markedly since then.

To investigate the function of CSP after transfusion in the context of dual antiplatelet therapy, endogenous platelets of healthy human study participants were inhibited with dual antiplatelet therapy (DAPT), as is done routinely for the prevention of early and late stent thromboses after coronary intervention in cardiac patients. DAPT includes drug-induced platelet function impairment by acetylsalicylic acid (ASA or aspirin) in combination with P2Y12 inhibitors, like clopidogrel. Out of all P2Y12 inhibitors only clopidogrel is currently indicated for stable ischemic heart disease. Bleeding rates between different P2Y12 inhibitors vary between 2.5 and 4.5% after stent placement, and bleeding can cause significant morbidity and mortality (Bang et al. EBioMedicine. 2017; 21:213-217) and offset some of the benefit of platelet inhibition (Levine et al. Journal of the American College of Cardiology. 2016; 68(10):1082-1115; Wallentin et al. The New England journal of medicine. 2009; 361(11):1045-1057).

ASA and clopidogrel remain the most commonly used DAPT combination for elective interventions and long-term DAPT after stent placement and no antidote is available (Michelson et al. Blood. 2017; 130(6):713-721). Both ASA and clopidogrel are irreversible inhibitors of platelet function indicating that a complete platelet population turnaround time is required to eliminate all antiplatelet effects (7-10 days) (Hennekens C H. Annual review of public health. 1997; 18:37-49; Hashemzadeh et al. The Journal of invasive cardiology. 2009; 21(8):406-412). Bleeding time studies have shown an additive effect on in vivo platelet inhibition by aspirin and clopidogrel (Payne et al. Journal of vascular surgery. 2002; 35(6):1204-1209).

To correlate platelet function after transfusion with platelet function after storage (i.e. before transfusion) in vitro, platelet function to agonists of pathways inhibited by DAPT, and adhesion and aggregation to extracellular matrix protein collagen and endothelial adhesion protein von Willebrand factor (VWF) were tested. To test platelet function in vivo, NOD-SCID mice were transfused with human platelets in the presence of human VWF, and a randomized, cross over study in healthy human subjects was performed.

Materials and Methods. Materials. The following items were purchased: Low molecular weight heparin (Lovenox, enoxaparin sodium; Sanofi-Aventis, Bridgewater, N.J.), heparin-coated capillaries (VWR, West Chester, Pa.), bovine serum albumin (BSA, fraction V, Sigma-Aldrich, St. Louis, Mo.), clopidogrel (Plavix, Sanofi-Aventis, France), acetylsalicylic acid (Sigma Aldrich, St. Louis, Mo.), anti-GPIbβ-488 (Emfret Analytics (Wuerzburg, Germany), PAC-1 and P-selectin antibodies (BD Biosciences, San Jose, Calif.), platelet agonists ADP, arachidonic acid, fibrillar collagen type I (Chronolog, Havertown, Pa.), Humate-P (CSL-Behring, King of Prussia, Pa.), calcein AM (Invitrogen, Carlsbad, Calif.), Drabkin's reagent (Sigma-Aldrich, St. Louis, Mo.), VASP-phosphorylation kit (STAGO, Asnieres sur Seine Cedex, France) thromboxane B2 ELISA (Cayman chemicals, Ann Arbor, Mich.). VerifyNOW® for aspirin and clopidogrel was a sent out test to the University of Washington clinical laboratory at UW Harborview (Seattle, Wash.).

Bleeding time assay. Bleeding time templates were sterilized before each usage. The assay was performed as described in Slichter et al. Blood. 2015; 126. In brief, disposable surgical blades (#11) made from sterile, stainless steel (Medline Industries, Inc, Mundelein, Ind.) were attached to the sterile bleeding time template. A sphygmomanometer was inflated to 40 mmHg on the left or right arm to allow arterial inflow, but block venous outflow. Template and blade were used to perform three scratches on the corresponding forearm with 1 mm and 9 mm length on volar side. The time required for cessation of bleeding was then measured by blotting with filter paper (Accriva Diagnostics, Inc, San Diego, Calif.) every 30 seconds without disturbing the wound. The test was stopped after 30 min and steri-strips were applied perpendicularly to the incision site to avoid scarring.

VerifyNOW®. Samples for VerifyNOW® were collected in citrate. Whole blood samples were sent to University of Washington Harborview labs.

Mice. NOD/SCID mice and WT (wild type) mice (C57/Bl6) were purchased from Jackson labs. Where indicated, NOD/SCID mice and WT mice (C57/Bl6) were treated with acetylsalicylic acid (aspirin) and clopidogrel (DAPT) 12 hours before the experiment with aspirin at a dosage of 5 mg/kg bodyweight and clopidogrel at 75 mg/kg bodyweight. COX-1 inhibition and P2Y12 inhibition was confirmed by standard aggregometry (impaired response to arachidonic acid and ADP, respectively, FIGS. 13A, 13B). Treatment with DAPT did not affect platelet counts or counts of other blood cells. Experimental procedures were approved by the controlling Animal Care and Use Committee.

Aggregometry. Platelets were isolated as platelet rich plasma, or washed and re-suspended at a concentration of 3×10⁸ platelets/ml in Tyrode's Buffer (137 mM NaCl, 0.3 mM Na₂HPO₄, 2 mM KCl, 12 mM NaHCO₃, 5 mM N-2-hydroxyethylpiperazine-N′-2-ethanesulfonic acid, 5 mM glucose, pH 7.3) containing 0.35% BSA and 1 mM CaCl₂). The experiment was performed at 37° C. under stirring conditions (1200 rpm). After addition of an agonist, light transmission was recorded over 7 min on a Chrono-log 4-channel optical aggregation system (Chrono-log, Havertown, Pa.).

Corrected count increment (CCI). CCI can be calculated by the following formula:

CCI=[Platelet increment (platelets/μL)×body surface area(m²)×10¹¹]/platelet dose (e.g., 3.0×10¹¹ to 4.0×10¹¹)

Preparation of plasma and human platelets. To generate platelet rich plasma (PRP), citrated whole blood was obtained and centrifuged at 190 g. After centrifugation, the platelet rich plasma was removed and the remaining sample (whole blood minus PRP) was centrifuged at 2000 g. The centrifugation allowed two layers to form: platelet poor plasma and red cells and white blood cells. To collect standard apheresis platelets for in vitro testing or autologous transfusion, a standard single apheresis platelet unit (target platelet yield 6.0×10¹¹/unit and concentration of 1500×10³ platelets/μL) was collected from 8 healthy subjects using the Trima Accel Automated Blood Collection System (TerumoBCT, Denver, Colo.). Each unit was calibrated to a final platelet concentration of 700-2100×10⁶ platelets/μL, as per allowable bag parameters. Each calibrated unit was allowed to rest at room temperature for 1 hour without agitation before transfer to cold storage at 4° C. for a predetermined period of up to 20 days, or was stored up to 5 days at RT (22° C.). The cold-stored bags were not agitated. RT-stored bags were agitated as per standard requirement for clinically used units.

αIIbβ3 activation/α-granule secretion. Platelets in platelet rich plasma were activated with ADP, arachidonic acid, or collagen in the presence of R-phycoerythrin-conjugated PAC-1 antibody for 10 minutes and studied immediately by flow cytometry. For P-selectin expression platelets were incubated with FITC-conjugated CD62P antibody (BD Biosciences).

Flow chamber studies. In vitro flow studies were performed in a microfluidic device purchased by Cellix (Dublin, Ireland). The flow chambers used had the following dimensions: width: 250 μm, height: 60 μm, length: 6 mm. The chambers were coated with collagen (fibrillar type I), at a concentration of 200 μg/mL and incubated at room temperature for 3 h. Murine whole blood was drawn from the retro orbital plexus into heparinized tubes (30 U/mL enoxaparin), incubated with 1.5 μg/mL of anti-GPIX (glycoprotein IX)-Alexa488 and infused at arterial (1500-S) wall shear rates for 5 minutes. Adhesion of platelets was monitored continuously with an Olympus inverted microscope IX80 (Olympus Instruments Inc, Tokyo, Japan) equipped with a Hamamatsu monochrome camera (Hamamatsu City, Japan). Images were analyzed using Slidebook software (3i, software, Denver, Colo.). Equal labeling verification was assessed by labeling fresh, room temperature-stored platelets with calcein AM and testing for fluorescence intensity by flow cytometry. All groups showed the same level of calcein green loading, i.e. equal labeling. An area of assessment was defined before perfusing the flow chambers and recording the video for analysis.

Tail bleeding assay. NOD/SCID mice (8 weeks old) were treated with DAPT as described above and matched for weight and anesthetized with 100 mg/kg ketamine (Forte Dodge Animal Health) and 10 mg/kg xylazine (Lloyd Laboratories) 10 minutes before tail transection. Other groups have shown contradictory results regarding the ability of human platelet GPIbα to interact with mouse VWF (Salles et al. Blood. 2009; 114(24):5044-5051; Chen et al. Nature biotechnology. 2008; 26(1):114-119). To promote human platelet function with human VWF at the site of injury, mice were perfused with Humate-P at concentrations equivalent to those given to humans with active bleeding 30 min prior to the experiment. Platelets were perfused 5 min prior to tail injury and 5 min after tail injury. Mice were placed on a heating pad, and tails were transected 5 mm from the tip with a razor blade and immediately immersed into a 20-mL scintillation vial filled with 10 mL Drabkin's reagent to promote hemolysis (prewarmed to 37° C.). Every 5 minutes 500 μL was taken from the vial to assess stepwise blood loss. The experiment was stopped after 30 min and the animal was euthanized. To measure blood loss volume, the collected blood sample was measured by a plate reader and the optical density (490 nm) was determined and compared with a standard curve. Time to occlusion is defined as the earliest time point when no change in hemoglobin concentration was recorded compared to later time points.

Healthy human subjects research. The research was approved by the Western Institutional Review Board (WIRB) and all human participants gave written informed consent. The study was conducted in accordance with the Declaration of Helsinki.

Statistical analysis. Based on a previous study which evaluated extended RT-storage with platelet additive solutions (Slichter et al. Blood. 2014; 123(2):271-280), an effect size of 2 was calculated for platelet recoveries between different time points. Based on this a sample size of n=7 was calculated to be required to provide 80% power to detect a statistically significant difference between cold-stored and RT-stored platelets (t-test, unpaired, two-tailed, a err. prob. 0.05). Therefore, the study included enrolling 7 subjects per group and then assessing for statistical significance. No additional data points were obtained after the study was concluded and tests for significance were run. Results are reported as mean±standard error of the mean and statistical significance was assessed by unpaired, 2-tailed Student t test, unless otherwise indicated. A P value equal or less than 0.05 was considered significant.

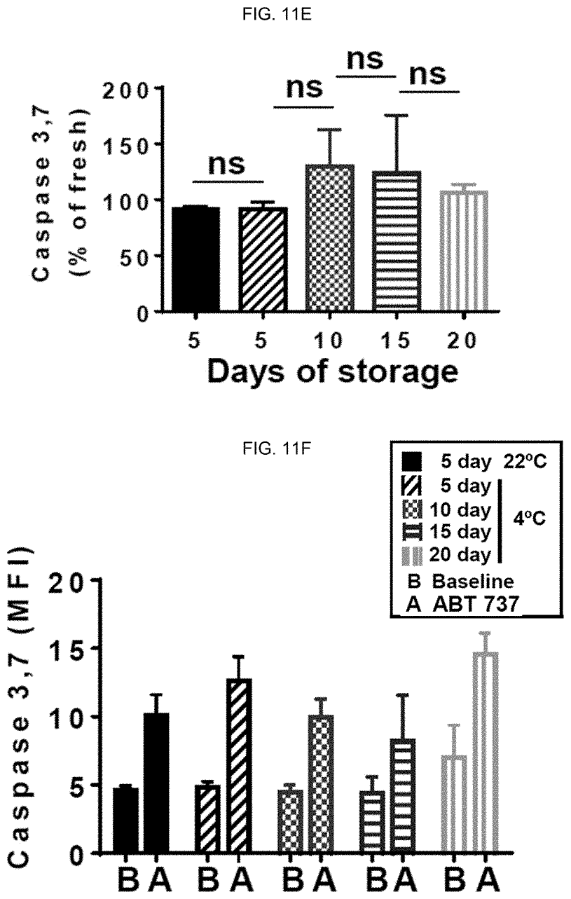

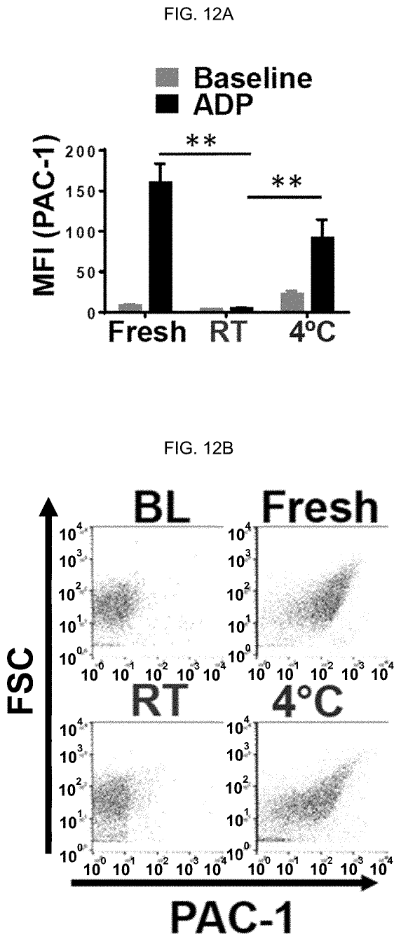

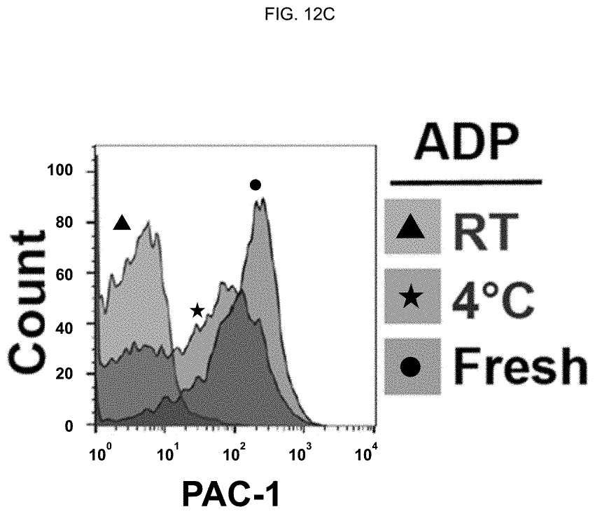

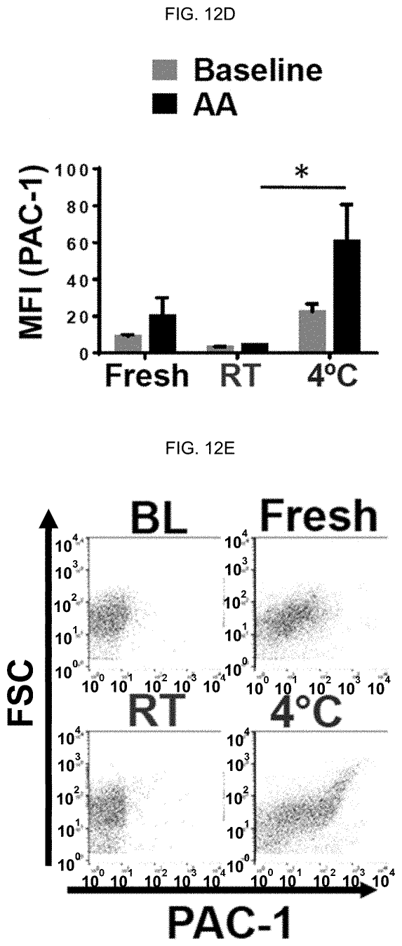

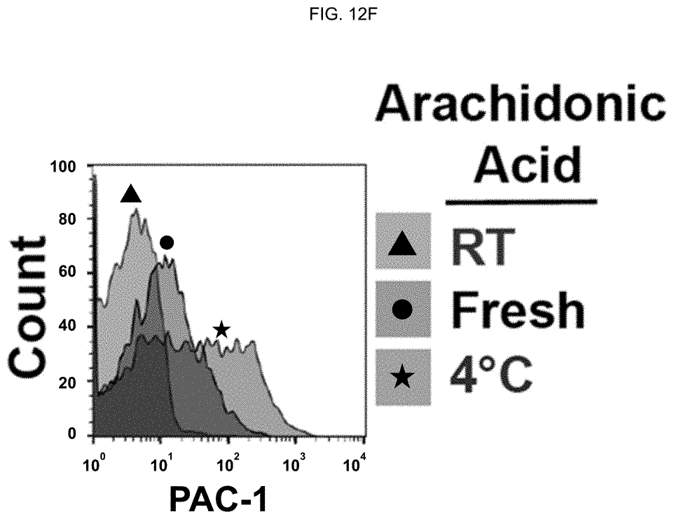

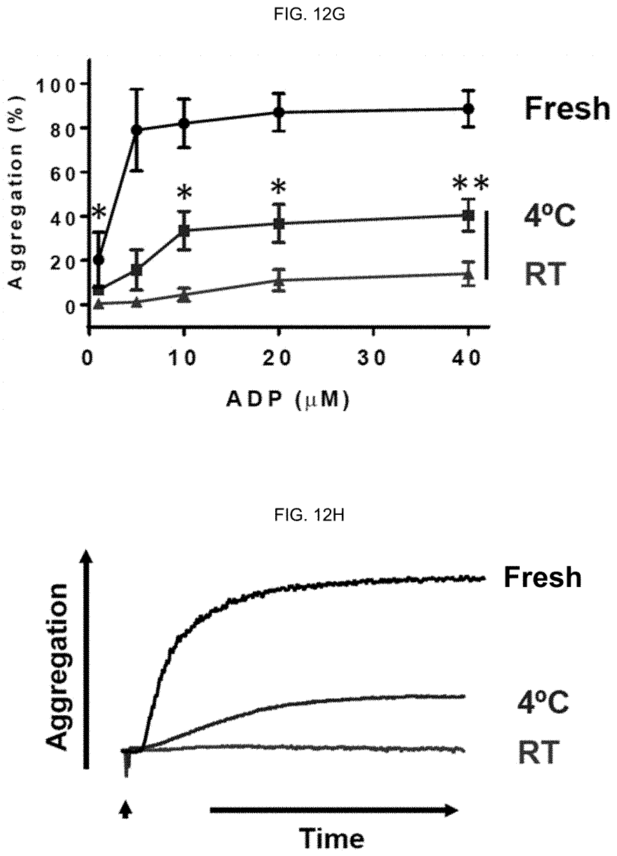

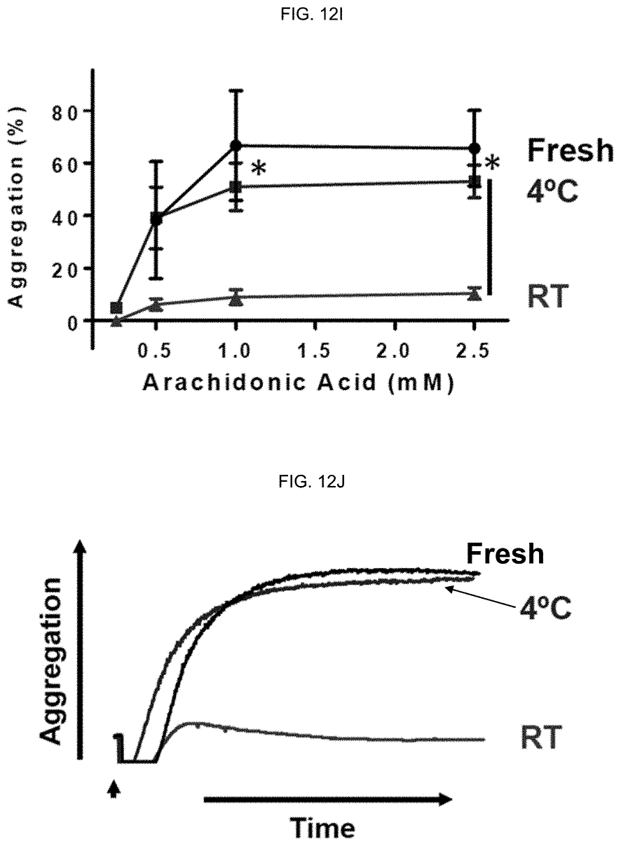

Results. Pre-transfusion platelet responses to pathways inhibited by DAPT. Apheresis platelets (yield of 3.0×10¹¹-4.0×10¹¹ platelets/bag; concentration of 0.7×10⁶-2.1×10⁶ platelets/μL plasma) were either stored for 5 days at RT with agitation as is currently done for clinical purposes or stored at 4° C. without agitation after a 1-hour rest period at room temperature as previously described (Stolla et al. Transfusion. 2018; 58(10):2407-2413). To test platelets' response through the P2Y12 pathway of platelet activation, platelets were stimulated with 40 μM ADP. Five day cold-stored platelets showed significantly more integrin activation when compared to 5-day RT-stored platelets (FIG. 12A). Platelets were stimulated with arachidonic acid (AA) to test their ability to activate αIIbβ3 integrins in the pathway inhibited by acetylsalicylic acid. Cold-stored platelets showed significantly more integrin activation compared to 5 day room temperature-stored platelets (FIG. 12D). Scatter plots and histograms showed a clear right-shift of PAC-1 binding at 4° C. comparable to fresh platelets with both agonists (FIGS. 12B, 12C, 12E, 12F). When arachidonic acid was used, the response of cold platelets exceeded the response of fresh platelets. Of note, extensive integrin pre-activation was observed in cold platelets and the extent of pre-activation reached the same level as arachidonic acid-stimulated fresh platelets.

To test platelet responses to varying doses of agonists of both DAPT-inhibited pathways platelets were incubated with increasing doses of either ADP (FIGS. 12G, 12H) or arachidonic acid (FIGS. 12I, 12J). The aggregation response was found to be significantly higher for 4° C. platelets compared to 22° C.-stored platelets for both agonists; however, response to arachidonic acid was almost indistinguishable and non-significantly different from the response of fresh platelets (FIGS. 12I, 12J). Without being bound by any one theory, it is hypothesized that cold-stored platelets are better suited to reverse antiplatelet therapy in vitro and in vivo.

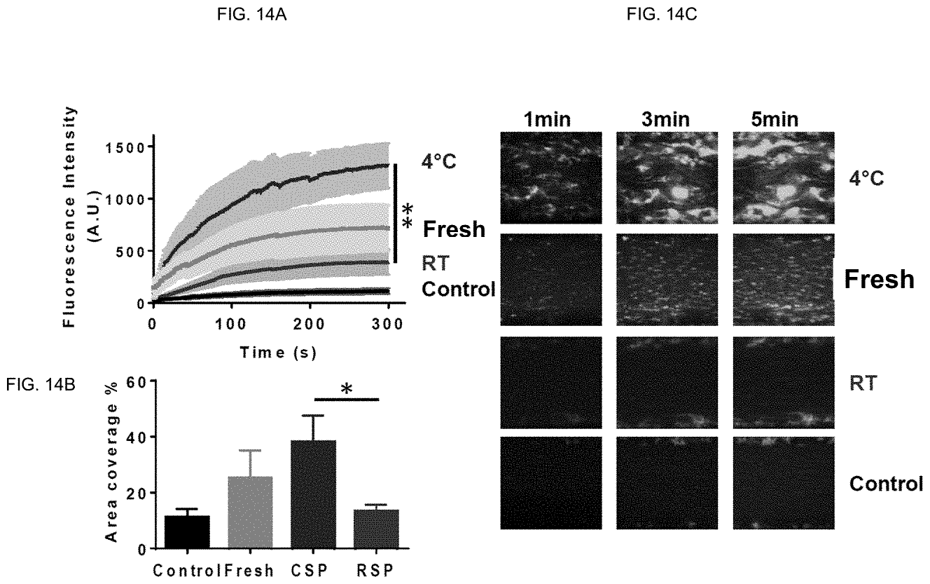

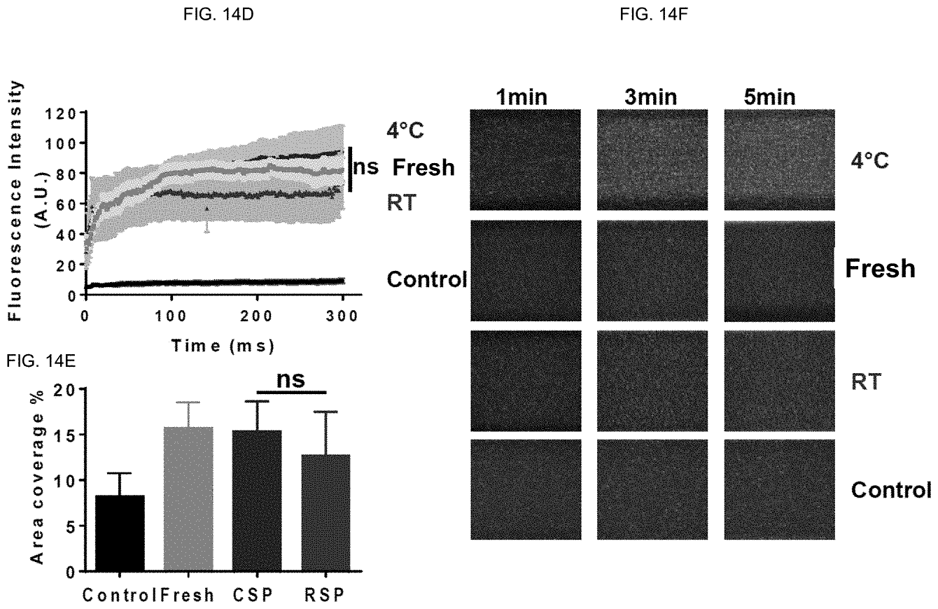

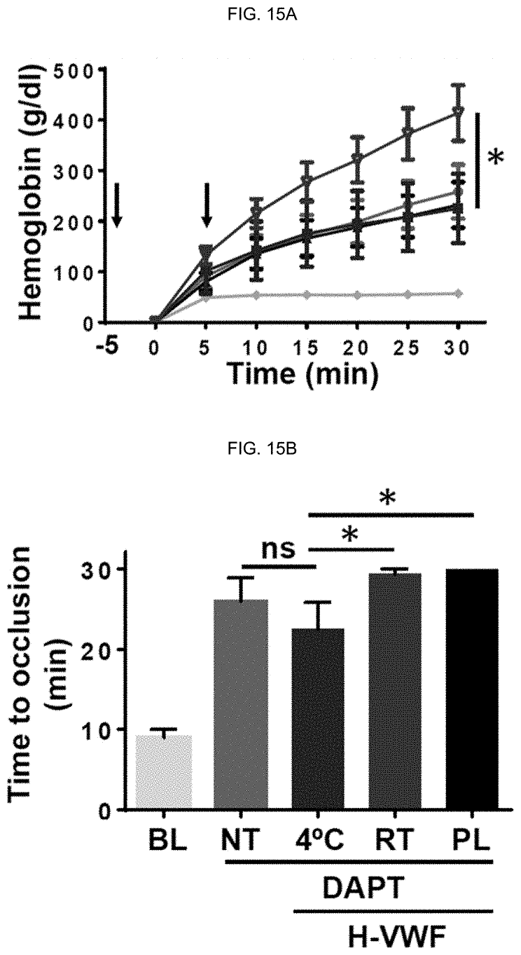

To test the function of cold-stored platelets under flow conditions in the setting of DAPT-reversal, DAPT-inhibited platelets were perfused over the extracellular matrix protein collagen. C57Bl6 mice were treated with DAPT and platelet inhibition in response to ADP and arachidonic acid was verified by aggregometry (FIGS. 13A, 13B). Either 5-day cold-stored, 5 day RT-stored platelets, or fresh platelets were added in a 1:3 ratio before perfusion to mimic 2 apheresis platelet unit transfusions in vitro. The mixed cell suspension was perfused for 5 minutes and mean fluorescence intensity and area coverage was recorded. Addition of 4° C.-stored platelets allowed the formation of larger three-dimensional thrombi compared to RT-stored platelets. This was reflected in a significantly higher mean fluorescence intensity and significantly higher area coverage compared to room temperature-stored platelets after 5 min of perfusion (FIGS. 14A, 14B). Both RT and 4° C. did not differ significantly from fresh platelets in area coverage (FIG. 14B). A monolayer of adherent platelets was observed in the control group (only DAPT inhibited, endogenous platelets without addition of exogenous platelets).

Another adhesive protein prevalent at the site of vascular injury is von Willebrand factor (VWF). The same groups as outlined above were perfused over human VWF (Humate-P). No significant differences were seen between any of the exogenously added platelet groups (FIGS. 14D-14F). Similar to the collagen perfusion experiments, the ability of DAPT-treated platelets to adhere to VWF was unaffected (FIGS. 14D-14F). Taken together, these data suggest that platelet function to pathways inhibited by DAPT is better preserved at cold storage compared to room temperature storage, and the ability to form thrombi under flow conditions is better preserved on collagen, compared to VWF.

Stored human platelets to reverse DAPT in vivo. To test the ability of RT or cold-stored human platelets in vivo, a hybrid xenotransfusion/tail injury model was developed based on a previously described model (Saito et al. International journal of experimental pathology. 2016; 97(3):285-292). NOD/SCID mice were treated with DAPT the same way as C57Bl6 were treated and inhibition of platelet responses to ADP and arachidonic acid was verified by aggregometry the same way. Human platelets, fresh, 5 day cold-stored, 5 day RT-stored were transfused 5 min prior and 5 min after tail cut and blood was collected over 30 min until the experiment was terminated. Surprisingly, RT platelet transfusions significantly increased blood loss compared to mice on DAPT without transfusion, while cold-stored platelets led to a small but not significant decrease in blood loss compared to DAPT-treated mice without transfusion at 5 minutes. Interestingly, mice transfused with an equivalent amount of plasma led to the same degree of reduction in blood loss at 5 minutes (FIGS. 15A, 15B). When time to occlusion was analyzed, a significantly shorter time to occlusion was observed when cold-stored platelets were transfused, compared to RT-stored platelets and plasma-transfused animals (FIG. 15B); however, again no significant difference was seen compared to DAPT-treated animals without transfusion.

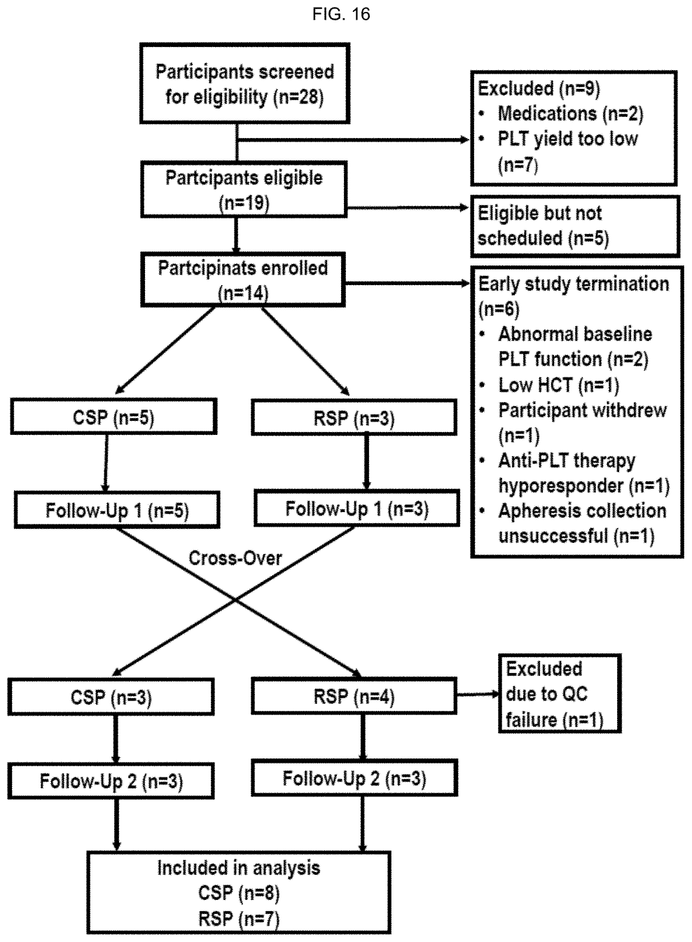

Stored platelet function in healthy human subjects treated with DAPT. Study participant demographics and recruitment. Of 8 subjects enrolled and randomized in the study all had evaluable primary outcome and secondary outcome data for cold-stored platelet transfusions. One of the subjects did not complete the RT-storage arm because of quality control failure (n=1) (FIG. 16). The baseline characteristics are described in Table 1 and an enrollment and study flow chart is shown in FIG. 16.

TABLE 1
Demographic characteristics of subjects completing the study

| | |
|---|---|
| Age, yr (IQR) | 28 (24, 32) |
| Female sex, no (%) | 2 (25) |
| Height (cm) ± STDEV | 173 ± 9.3 |
| Weight (kg) ± STDEV | 70.6 ± 4.7 |
| BMI ± STDEV | 23.8 ± 2.6 |
| washout (d) ± STDEV | 15 ± 4.9 |
| CSP first (%) | 5 (62.5) |

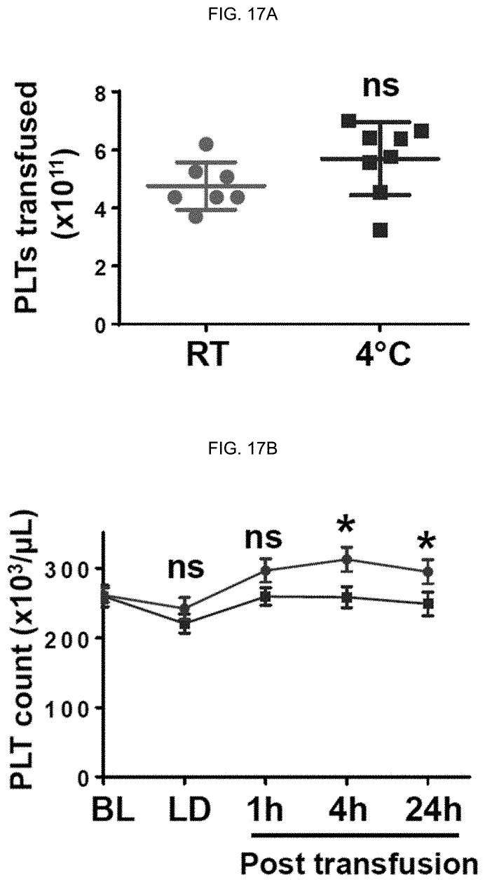

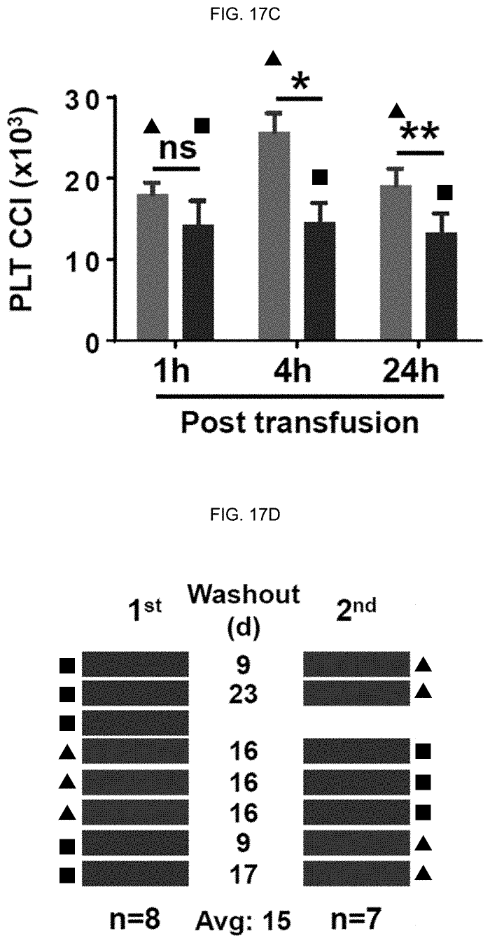

Unit characteristics and safety assessment. All cold-stored units passed quality control assessments after storage. One RT-stored unit had to be discarded because of a low pH and concern for bacterial contamination. Both 4° C.-stored and 22° C.-stored platelet transfusions were tolerated well by the recipients. Six hours after transfusion of a cold-stored unit one subject had an adverse event which was considered to be unrelated to transfusion. The absolute amount of platelets did not differ significantly between the two study arms, but there was a trend for a higher transfused platelet count in the cold-storage group (4.8×10¹¹ vs. 5.7×10¹¹, p=0.1) (FIG. 17A).

Platelet transfusion efficacy parameters. Platelet counts dropped in all subjects after collection of the double apheresis unit. Both arms did not differ significantly between pre-transfusion counts, and 1 h post transfusion counts, but platelet counts were significantly lower in the CSP arm after 4 h and 24 h (FIG. 17B). Correspondingly, the corrected count increments (CCI) after transfusion did not differ significantly after 1 h post transfusion, but were significantly lower at 4 hours and 24 hours after transfusion of CSP (FIG. 17C). Overall, five subjects were randomized to receive CSP first and three to receive RSP first. Due to one quality control failure in the RSP group, the second round of transfusion included only 3 in the RSP group and 4 in the CSP group (FIG. 17D). The average time between the two rounds was 15 days, and the shortest time between two rounds was 9 days (FIG. 17D).

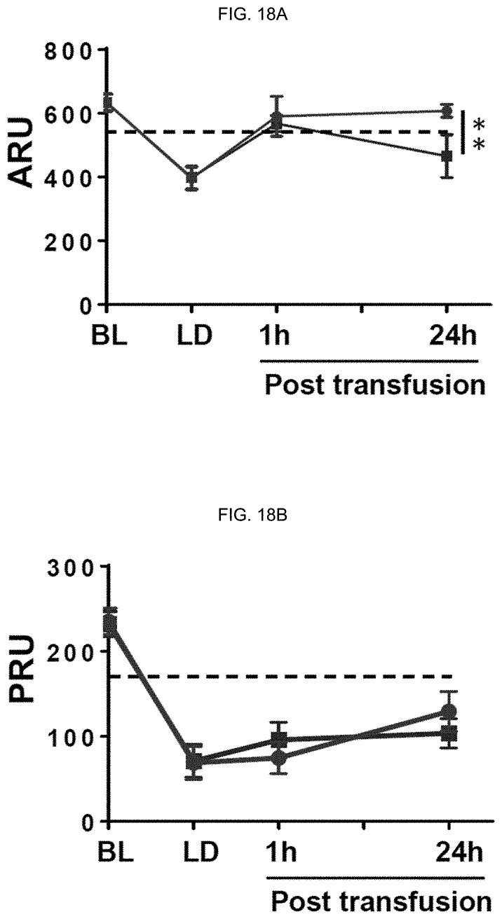

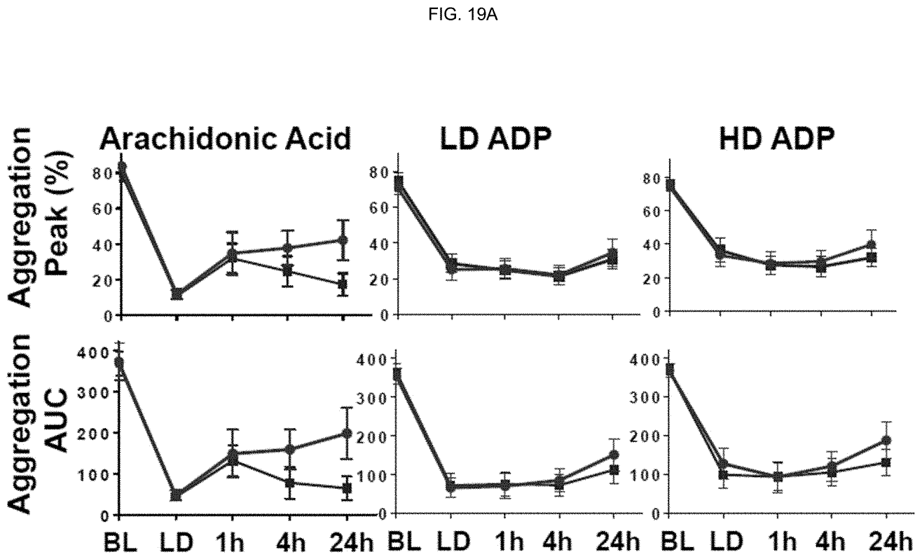

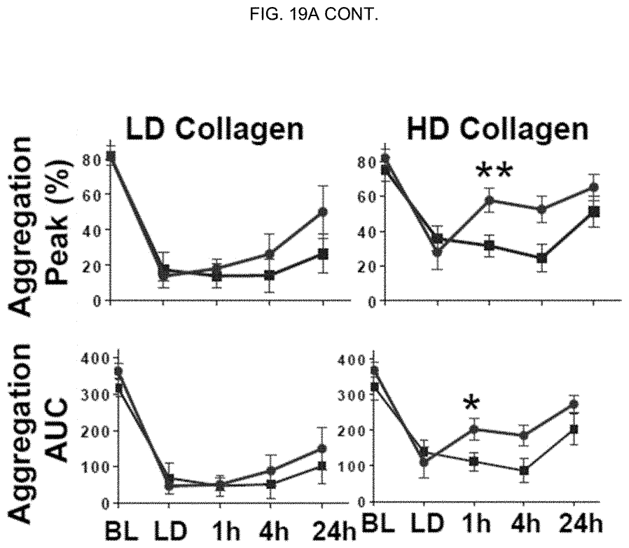

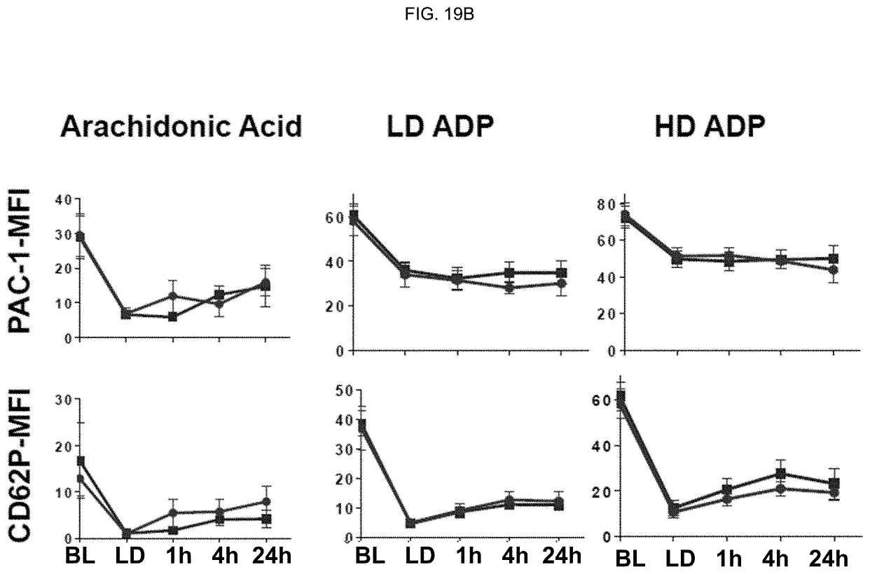

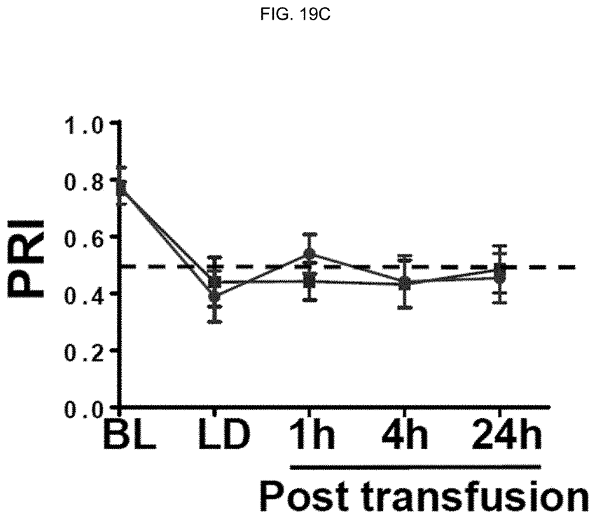

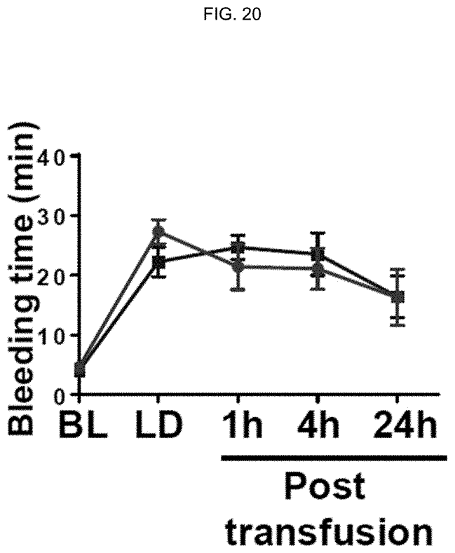

Platelet Function Testing after Transfusion. The first primary endpoint, platelet function testing with the VerifyNOW® assay, a whole blood, point of care test, based on the ability of activated αIIbβ3 integrin on platelets to bind fibrinogen-coated microparticles after addition of pathway-specific agonists showed reversal of the effect of acetylsalicylic acid with both products at 1 h and 4 h post transfusion, but platelet inhibition re-appeared after 24 h post transfusion in the cold-storage arm, and there was a significantly higher ARU (aspirin reaction units) in RT-stored platelets at this time point (FIG. 18A). No significant differences were observed between the two products in the VASP phosphorylation assay for clopidogrel (FIG. 18B). There was no evidence of any significant reversal with both products. The second primary endpoint was defined as platelet response to agonists for pathways inhibited by DAPT measured by flow cytometry αIIbβ3 integrin activation (PAC-1 antibody binding) and α-granule secretion (P-selectin antibody binding). There were no significant difference between CSP and RSP transfused subject's platelet function in these assays, but there was a trend for a higher degree of integrin activation and α-granule secretion in subjects transfused with RSP in response to arachidonic acid at the 1 h post transfusion time point (FIG. 19A) and conversely, a higher level of integrin activation at 4 h in response to 5 μM ADP (FIG. 19A) and a trend for a higher degree of α-degranulation at the 4 h time point at the 4 h time point (FIG. 19B). The template bleeding time was defined as secondary endpoint and overall no significant differences were observed between the two groups (FIG. 20). Surprisingly, platelets isolated from recipients of RT-stored platelets aggregated significantly better in response to collagen compared to platelets isolated from recipients of cold-stored platelets at 1 h and 4 h post transfusion (FIG. 19A). But no significant differences were observed between the aggregation responses to arachidonic acid and ADP. VASP-phosphorylation, a test commonly used clinically to test for response to clopidogrel suggested a trend for a higher platelet reactivity index (PRI) at 1 h after transfusion with RT-stored platelets at 1 h after transfusion, but at 4 h and 24 h no difference was apparent between the two groups (FIG. 19C). To test which platelet transfusion group can restore the thromboxane release in response to arachidonic acid, platelets were stimulated in the aggregometer and the supernatant analyzed for thromboxane B2 by ELISA.

Summary of Results. Functional platelet assays under static, turbulent flow, laminar flow, and in vivo conditions suggest superior platelet function after cold-storage compared to room temperature storage. In healthy human subjects, no significant difference was detected after transfusion of RT-stored or cold-stored platelets when the bleeding time was assessed and in platelet function assays with agonists for pathways inhibited by dual antiplatelet therapy. VerifyNOW® for acetylsalicylic acid and aggregometry with arachidonic acid indicated that after 1 h-4 h of transfusion the effect of acetylsalicylic acid was reversed, but the limited survival of cold-stored platelets led to a reappearance of inhibition by acetylsalicylic acid 24 h after transfusion. Stimulation with collagen led to significantly increased aggregation after transfusion with RT-stored platelets at 1 h and 4 h after transfusion.

Discussion. The present study was based on observations that CSP show better in vitro responses to agonists even up to 20 days of storage. It is currently unknown if in vitro function predicts in vivo function in actively bleeding patients or patients at risk for bleeding. Therefore, it was hypothesized that stored platelet in vitro function predicts post transfusion function in a mouse model of active bleeding in mice and post transfusion platelet function in healthy human subjects on DAPT. The data demonstrate superior platelet function post cold-storage in flow cytometry, aggregometry, and in a flow chamber DAPT reversal assay. Mice after tail cut injury show significantly less blood loss after CSP transfusion, but no significant difference is seen between CSP, volume control, and no transfusion. In fact, while CSP did not show improvement to controls, RT storage led to more blood loss compared to controls and CSP. These results are in agreement with a recent clinical trial investigating the effect of RSP to reverse DAPT in a patient population with spontaneous intracranial hemorrhage: RSP worsened outcomes compared to no transfusion (Baharoglu et al. Lancet. 2016; 387(10038):2605-2613). Pro-inflammatory cytokines in RT-stored units and possible volume effects are offered as explanations (Baharoglu et al. Lancet. 2016; 387(10038):2605-2613; Senzel et al. American journal of clinical pathology. 2019; 152(1):1-6). Similarly, the present study shows increase in blood loss after RSP transfusion, suggesting a possible downside of transfusion of RSP, possibly an equally dysfunctional platelet population competing with the DAPT inhibited platelets. A xeno-transfusion model was utilized in the present study by transfusing human platelets into immunocompromised mice. Blood loss and time to occlusion were the read-outs in the mouse model, while the PATCH trial (human trial) utilized neurologic outcomes by a functional score and imaging to assess cerebral health in severely compromised patients with intracranial hemorrhage. Mice were injected with human VWF to help human platelet GPIbα-mouse VWF interaction at the site of injury since some previous reports questioned the ability of mouse VWF to engage human GPIbα (Salles et al. Blood. 2009; 114(24):5044-5051; Chen et al. Nature biotechnology. 2008; 26(1):114-119). Nevertheless, the present findings offer a possible explanation for the unexpected finding of the PATCH trial: impaired hemostasis after transfusion of RSP possibly due to competing populations of inhibited platelets to the extracellular matrix (ECM) binding sites of injury. Fittingly, a volume-independent flow chamber model showed clear superiority of cold-stored platelets to adhere to collagen under flow conditions. In fact, the flow chamber model even showed superior adhesion and aggregation of CSP compared to fresh platelets.

To further investigate if these preclinical in vitro and animal model findings were translatable into human subjects, a small, randomized, cross over trial was performed to correlate post-storage function with post transfusion function in healthy human subjects. Previously published data indicate that CSP are mostly cleared within 24 h. This is why CSP were abandoned in the 1960s and 1970s. Therefore, it was expected that a decrease in circulating platelets and a decrease in platelet function in the CSP arm at the 24 h time point would be seen. This also served as an internal control, because as shown in the CSP group, it is not the generation of new platelets that is responsible for a reversal of DAPT at this 24 h time point in the VerifyNOW® data set (36-48 h after leading dose). Curiously, RSP responded better to collagen by aggregometry at 1 h and 4 h of transfusion. This is noteworthy due to multiple reasons: Firstly, platelet response to collagen, while generally dampened by the lack of reinforcement of activation by second wave mediators like thromboxane A2 and ADP, should be dampened by DAPT, but is not directly inhibited by DAPT. Secondly, especially the early hours after transfusion have been shown to be favorable for CSP in studies from the time of initial evaluation of cold-storage.

While acetylsalicylic acid and clopidogrel prolong the bleeding time to an extent comparable to severely thrombocytopenic patients, acetylsalicylic acid alone is a rather weak platelet function inhibitor and leads to only a minor increase in the bleeding time. Therefore, inhibition of platelet function by acetylsalicylic acid and platelet function testing by the bleeding time may not allow differentiation between minor quality differences of transfusion products. Conversely, the effect of the P2Y12 inhibitor clopidogrel was not overcome by either study product at no time point. RT-stored platelets showed a higher platelet reactivity index 1 h after transfusion, but this was not significantly different compared to CSP. These data are in partial agreement with a study by Pruller et al. who showed higher PRI after transfusion of RSP at early time points, but the Pruller study showed further improvement after 4 h and 24 h, while the present study shows a return to platelet inhibition at 4 h and 24 h.

Further studies are needed to investigate CSP in patients with dysfunctional platelets either due to antiplatelet therapy or due to acquired or congenital disorders.

The Examples are provided herein to demonstrate that cold-stored platelet products of the disclosure are effective in settings where functional platelets are needed, as assessed by ex vivo and in vivo assays, and suggest that the cold-stored platelet products described herein have clinical utility.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant increase in clot formation of platelet samples 3 days, 5 days, 10 days, 15 days, and/or 20 days during or after cold storage.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

What is claimed is:

1. A method of forming a cold-stored platelet product comprising:
collecting platelets and plasma from a subject;
calibrating the platelets to a concentration of 0.5×10⁶-2.3×10⁶ platelets/μL of the plasma to create a platelet sample;
maintaining the platelet sample at room temperature for 1 hour without agitation; and
transferring the platelet sample to cold storage for 3-20 days after the maintaining, wherein the cold storage is 4±2° C. and without agitation, thereby forming a cold-stored platelet product.

2. The method of claim 1, wherein the collecting is by apheresis.

3. The method of claim 1, wherein the collecting of platelets is to a yield of 3.0×10¹¹-4.0×10¹¹ platelets/bag.

4. A method of forming a cold-stored platelet product comprising:
collecting platelets and plasma from a subject;
calibrating the platelets to a concentration of 0.5×10⁶-2.3×10⁶ platelets/μL of the plasma to create a platelet sample;
maintaining the platelet sample at room temperature for 20 minutes to 2 hours; and
transferring the platelet sample to cold-storage for a period of time, thereby forming a cold-stored platelet product.

5. The method of claim 4, wherein the collecting is by apheresis.

6. The method of claim 4, wherein the collecting of platelets is to a yield of 3.0×10¹¹-4.0×10¹¹ platelets/bag.

7. The method of claim 4, wherein the concentration is 0.7×10⁶-2.1×10⁶ platelets/μL of the plasma.

8. The method of claim 4, wherein the maintaining the platelet sample at room temperature is for 1 hour.

9. The method of claim 4, wherein the maintaining the platelet sample at room temperature is without agitation of the platelet sample.

10. The method of claim 4, wherein room temperature is 20-24° C.

11. The method of claim 4, wherein the cold storage is 4±2° C.

12. The method of claim 4, wherein the period of time is 3-20 days.

13. The method of claim 4, wherein the cold storage is without agitation.

14. The method of claim 4, wherein the cold-stored platelet product is clot-free for up to 20 days at cold storage.

15. The method of claim 4, wherein the platelet product is a transfusion ready platelet product.

16. A population comprising a plurality of the cold-stored platelet product formed by the method of claim 4, wherein each cold-stored platelet product is a unit and wherein 97% or more of the units remain free of macro-aggregates for at least 10 days after cold storage begins.

17. The population of claim 16, wherein the population has at least 20 units.

18. The population of claim 16, wherein the population has at least 50 units.

19. The population of claim 16, wherein no more than 3 units comprise macro-aggregates.

20. The population of claim 16, wherein the platelet products are transfusion ready platelet products.
